(19) United States
(12) Patent Application Publication
(10) Pub. No.: US 2025/0000976 A1
(43) Pub. Date: Jan. 2, 2025

(54) ANTI-BCMA SINGLE DOMAIN ANTIBODIES AND THERAPEUTIC CONSTRUCTS

(71) Applicant: NATIONAL RESEARCH COUNCIL OF CANADA, Ottawa, Ontario (CA)

(72) Inventors: Mehdi ARBABI-GHAHROUDI, Ottawa, Ontario (CA); Risini WEERATNA, Ottawa, Ontario (CA); Scott MCCOMB; Cunle WU, Montreal, Quebec (CA)

(21) Appl. No.: 18/698,911

(22) PCT Filed: Oct. 5, 2022
(86) PCT No.: PCT/CA2022/051473

§ 371 Date: Apr. 5, 2024

Related U.S. Application Data
(60) Provisional application No. 63/253,386, filed on Oct. 7, 2021.

(51) Int. Cl. A61K 39/00; A61P 35/00; C07K 14/725; C07K 16/28

(52) U.S. Cl. CPC A61K 39/464416; A61K 39/4611; A61K 39/4631; A61P 35/00; C07K 14/7051; C07K 16/2809; C07K 16/2878; C07K 2317/24; C07K 2317/31; C07K 2317/622

(57) ABSTRACT

Herein are provided anti-BCMA single domain antibodies (sdAb) prepared byte immunizing a llama with the ecto-domain of human B-cell maturation antigen (BCMA) that is preferentially expressed by mature B lymphocytes. By constructing a library of the heavy chain repertoire generated, VHH antibodies specific to the immunogen were isolated. The 13 unique example antibodies initially produced comprise CDR1, CDR2, and CDR3 sequences corresponding, respectively to SEQ NOs: 1-3, 4-6, 7-9, 10-12, 13-15, 16-18, 19-21, 22-24, 25-27, 28-30, 31-33, 34-36, 37-39; and related sequences. Also provided are multivalent antibodies comprising any one of the sdAbs, including bispecific T-cell engagers, bispecific killer cell engagers (BiKEs), and trispecific killer cell engagers (TriKEs). Also described are chimeric antigen receptors (CARs) for CAR-T therapy comprising any one of the aforementioned sdAbs. Uses of these molecules in the treatment of cancer or autoimmune diseases are also described, in particular hematological malignancies, such as multiple myeloma.

### CROSS REFERENCE TO RELATED APPLICATIONS

This application claims the benefit of priority of U.S. Provisional Application No. 63/253,386 entitled “ANTI-BCMA SINGLE DOMAIN ANTIBODIES AND THERAPEUTIC CONSTRUCTS” and filed on Oct. 7, 2021, the contents of which are herein incorporated by reference.

### FIELD

The present disclosure relates generally to anti-B-cell maturation antigen (BCMA) antibodies. More particularly, the present disclosure relates to anti-BCMA single domain antibodies.

### BACKGROUND

Cancer is a major public health problem and the second leading cause of death worldwide. Traditional therapy for cancer has included surgery, radiation and chemotherapy. These have been moderately successful for treatment of some cancers, particularly those diagnosed at early stages. However effective therapy is lacking for many aggressive cancers. for example, despite considerable advances in the treatment of multiple myeloma (MM) in the last decade, a substantial proportion of patients have short duration of response to these therapies and eventually become resistance to these therapies and succumb to the disease. Currently there is no cure for relapsed/refractory MM and hence a great unmet need exists for safe and efficacious MM treatments that can offer durable responses.

Immunotherapy; harnessing patients own immune system to recognize and kill cancer is now considered the fourth pillar of cancer therapy alongside with surgery, radiation and chemotherapy. Immunotherapy has shown great clinical efficacy in a number of hard to treat solid tumor malignancies. However, overall, the greatest success of immunotherapy to date has been in treating hematologic malignancies, in particular with the use of bi-specific T cell engager therapy and engineered cell therapy for treating relapsed and refractory acute lymphoblastic leukemia (ALL) and non-Hodgkin lymphoma (NHL).

Immunotherapy approaches for MM exists with the approval of two monoclonal antibodies targeting CD38 (daratumumab) and SLAMF7 (elotuzumab) for treatment of MM in 2015. However, both these antigen targets are also expressed on normal tissues including hematopoietic lineages and immune effector cells limiting their long term use.

It is, therefore, desirable to provide immunogenic molecule with affinity for cell markers relevant to MM and other diseases.

### SUMMARY

It is an object of the present disclosure to obviate or mitigate at least one disadvantage of previous.

In a first aspect, the present disclosure provides an isolated single domain antibody (sdAb), which binds specifically to human B-cell maturation antigen (BCMA), the sdAb comprising:
a) a CDR1 amino acid sequence GX₁X₂X₃X₄YX₅FV (SEQ ID NO: 59), wherein:
   X₁ is R or H,
   X₂ is A or T,
   X₃ is T or S,
   X₄ is D, N, or K, and
   X₅ is H, N, or Q,
a CDR2 amino acid sequence RX₆WX₇GX₈X₉P (SEQ ID NO: 60), wherein:
   X₆ is V or F,
   X₇ is S or G,
   X₈ is G, or S, and
   X₉ is S or T, and
a CDR3 amino acid sequence AATKDIX₁₀SRX₁₁YX₁₂Y (SEQ ID NO: 61), wherein:
   X₁₀ is M or L,
   X₁₁ is S or G, and
   X₁₂ is D or V;
b) a CDR1 amino acid sequence GX₁X₂FGX₃X₄X₅ (SEQ ID NO: 62), wherein:
   X₁ is S or D,
   X₂ is I, S, or G,
   X₃ is T or A,
   X₄ is Y or H, and
   X₅ is N, A, or V,
a CDR2 amino acid sequence ISSAGX₆T (SEQ ID NO: 63), wherein:
   X₆ is N or S, and
a CDR3 amino acid sequence NGAPWADX₇X₈VKVX₉N (SEQ ID NO: 64), wherein:
   X₇ is A or E,
   X₈ is E or P, and
   X₉ is Y or W;
c) a CDR1 amino acid sequence GX₁X₂X₃X₄X₅X₆X₇ (SEQ ID NO: 65), wherein:
   X₁ is N, S, or D,
   X₂ is S, I, or P,
   X₃ is F or I,
   X₄ is G, D, or T,
   X₅ is A, V, or T,
   X₆ is Y or A, and
   X₇ is N or T,
a CDR2 amino acid sequence ISSX₈GX₉T (SEQ ID NO: 66), wherein:
   X₈ is T or A, and
   X₉ is N, T, or S, and
a CDR3 amino acid sequence NGAPWGDX₁₀X₁₁VKVX₁₂X₁₃ SEQ ID NO: 67), wherein:
   X₁₀ is D or A,
   X₁₁ is P or L,
   X₁₂ is W or E, and
   X₁₃ is S, D, T, or N;

d) a CDR1 amino acid sequence as set forth in SEQ ID NO: 31,
a CDR2 amino acid sequence as set forth in SEQ ID NO: 32, and
a CDR3 amino acid sequence as set forth in SEQ ID NO: 33 (from hBCMA VcMRo8 (VF7/VF8));
or
e) a CDR1 amino acid sequence as set forth in SEQ ID NO: 34,
a CDR2 amino acid sequence as set forth in SEQ ID NO: 35, and
a CDR3 amino acid sequence as set forth in SEQ ID NO: 36 (from hBCMA-A3).

In one aspect, there is provided an isolated single domain antibody (sdAb), which binds specifically to human BCMA, the sdAb comprising:

A)
a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7 or hBCMA-2C3),
a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2 or hBCMA-4D1),
a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3),
a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5),
a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4),
a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1),
a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2),
a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6),
a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)),
a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2),
a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)),
a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10), or
a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2).

In one aspect, there is provided an isolated single domain antibody (sdAb), which binds specifically to human BCMA, the sdAb comprising:

A)
a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7 or hBCMA-2C3),
a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2 or hBCMA-4D1),
a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3),
a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5),
a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4),
a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1),
a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2),
a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6),
a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)),
a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2)
a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)),
a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10), or
a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2); or

B)
CDR1, CDR2, and CDR3 amino acid sequences that are at least 80% identical to the CDR1, CDR2, and CDR3 sequences defined in any one of part A) i) to xxviii).

In one aspect, there is provided an isolated single domain antibody (sdAb), which binds specifically to human BCMA, the sdAb comprising:
a CDR3 amino acid sequence as set forth in SEQ ID NO: 3,
a CDR3 amino acid sequence as set forth in SEQ ID NO: 6,
a CDR3 amino acid sequence as set forth in SEQ ID NO: 9,
a CDR3 amino acid sequence as set forth in SEQ ID NO: 12,
a CDR3 amino acid sequence as set forth in SEQ ID NO: 15,
a CDR3 amino acid sequence as set forth in SEQ ID NO: 18,
a CDR3 amino acid sequence as set forth in SEQ ID NO: 21,
a CDR3 amino acid sequence as set forth in SEQ ID NO: 24,
a CDR3 amino acid sequence as set forth in SEQ ID NO: 27,
a CDR3 amino acid sequence as set forth in SEQ ID NO: 30,
a CDR3 amino acid sequence as set forth in SEQ ID NO: 33,
a CDR3 amino acid sequence as set forth in SEQ ID NO: 36, or
a CDR3 amino acid sequence as set forth in SEQ ID NO: 39.

In one aspect, there is provided a VHH single domain antibody (sdAb) that competes for specific binding to BCMA with one of the isolated sdAbs described above.

In one aspect, there is provided a VHH single domain antibody (sdAb) that competes for specific binding to BCMA with one of the isolated sdAbs described above

In one aspect, there is provided a recombinant polypeptide comprising one or more sdAb as defined herein.

In one aspect, there is provided the sdAb defined herein fused to a human Fc (termed a “V_{H}H:Fc fusion”).

In a further aspect, the present disclosure provides anti-BCMA sdAb as defined herein linked to a cargo molecule.

In aspect, there is provided a nucleic acid molecule encoding an sdAb, the recombinant polypeptide, or the V_{H}H:Fc fusion as defined herein.

In one aspect, there is provided a composition comprising an sdAb as defined herein, or a polypeptide comprising such an sdAb; together with an acceptable excipient, diluent or carrier.

In one aspect, there is provided a use of the sdAb as defined herein or of an antibody comprising one or more V_{H}H:Fc fusion as defined herein for treatment of a cancer or an auto-immune disease.

In one aspect, there is provided a use of the sdAb as defined herein or of an antibody comprising one or more V_{H}H:Fc fusion as defined herein for preparation of a medicament for treatment of a cancer or an auto-immune disease.

In one aspect, there is provided the sdAb as defined herein or of an antibody comprising one or more V_{H}H:Fc fusion as defined herein for use in treatment of a cancer or an auto-immune disease.

In one aspect, there is provided a method of treating a cancer or an auto-immune disease in subject comprising administering to the subject the sdAb as defined herein or of an antibody comprising one or more V_{H}H:Fc fusion as defined herein.

In one aspect, there is provided a multivalent antibody comprising an sdAb as defined above.

In aspect, there is provided a recombinant nucleic acid molecule encoding the multivalent antibody as defined herein.

In one aspect, there is provided a composition comprising a multivalent antibody as defined herein; together with an acceptable excipient, diluent or carrier.

In one aspect, there is provided a use of the multivalent antibody as defined herein for treatment of a cancer or an auto-immune disease.

In one aspect, there is provided a use of the multivalent antibody as defined herein for preparation of a medicament for treatment of a cancer or an auto-immune disease.

In one aspect, there is provided the multivalent antibody as defined herein for use in treatment of a cancer or an auto-immune disease.

In one aspect, there is provided a method of treating a cancer or an auto-immune disease in subject comprising administering to the subject the multivalent antibody as defined herein.

In one aspect, there is provided a chimeric antibody receptor (CAR), which binds to human BCMA, comprising the VHH sdAb as defined herein.

In one aspect, there is provided a nucleic acid molecule encoding the CAR as defined herein.

In one aspect, there is provided a vector comprising the recombinant nucleic acid molecule as defined herein.

In one aspect, there is provided a recombinant viral particle comprising the recombinant nucleic acid as defined herein.

In one aspect, there is provided a cell comprising the recombinant nucleic acid molecule as defined herein.

In one aspect, there is provided an engineered cell expressing at the cell surface membrane the CAR as defined herein.

In one aspect, there is providing a use of the nucleic acid, vector, or viral particle as described herein for preparation of cells for CAR-T.

In one aspect, there is providing a method of preparing cells for CAR-T comprising contacting a T-cell with the viral particle as described herein. In one embodiment, the T-cell is from a donor. In one embodiment, the T-cell is from a patient.

In one aspect, there is providing a method of preparing cells for CAR-T comprising introducing into a T-cell the nucleic acid or vector as described herein. In one embodiment, the T-cell is from a donor. In one embodiment, the T-cell is from a patient.

In one aspect, there is provided a use of the CAR or of the engineered cell as described herein for treatment of a cancer or an auto-immune disease.

In one aspect, there is provided a use of the CAR or of the engineered cell as described herein for preparation of a medicament treatment of a cancer or an auto-immune disease.

In one aspect, there is provided the CAR or the engineered cell as described herein for use in treatment of a cancer or an auto-immune disease.

In one aspect there is provided a method of treating a cancer or an auto-immune disease in a subject, comprising administering to the subject the engineered cell as defined herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 depicts the structure of human BCMA molecule (known also as tumor necrosis factor receptor superfamily member 17; TNFRSF17). The amino acid sequence alignment of extracellular domain of both human and mouse are aligned to display the difference in nature and number of amino acid residues.

FIG. 2 depicts a SDS-PAGE of Protein A (MabSelect™ SuRe™) and IMAC-purified BCMA extracellular domain fusion proteins (mIgG2a-BCMA, hBCMA-ECD-FC5VHH and mBCMA-ECD-FC5VHH) under reducing and non-reducing conditions.

FIG. 3 depicts the llama heavy chain immune response from a test bleed (7 days post 3^{rd} immunization) and the final bleed (7 days post 5^{th} immunization) against BCMA-ECD.

FIG. 4 depicts the SDS-PAGE of 13 anti-BCMA VHH antibodies expressed in BL21(DE3) E. coli and purified by IMAC.

FIG. 5A depicts partial sdAb sequences parsed according to the IMGT numbering system.

FIG. 5B is a continuation of FIG. 5A, and depicts the remaining sequence of each sdAb.

FIG. 6A depicts binding of anti-BCMA VHH to tumor cell lines with high (RPM18226) BCMA expression.

FIG. 6B depicts binding of anti-BCMA VHH to tumor cell lines with low (Raji) BCMA expression.

FIG. 6C depicts binding of anti-BCMA VHH to tumor cell lines with no (Jurkat) BCMA expression.

FIG. 7A depicts competitive binding data by SPR (sdAbs A6 & H2).

FIG. 7B depicts competitive binding data by SPR (sdAbs A6 & H4).

FIG. 7C depicts competitive binding data by SPR (sdAbs A6 & VcMRo8).

FIG. 7D depicts competitive binding data by SPR (sdAbs H2 & H4).

FIG. 7E depicts competitive binding data by SPR (sdAbs H2 & VcMRo8).

FIG. 7F depicts competitive binding data by SPR (sdAbs H4 & VcMRo8).

FIG. 7G depicts a chart summarizing competitive binding data from FIGS. 7A to 7G.

FIG. 8 depict the sequence of the ecto-domain of the hBCMA and the positions of the disulfide bonds, and the yeast surface display constructs expressing the various BCMA fragments used cell ELISA for epitope mapping.

FIG. 9 depicts a chart showing the yeast cell ELISA measured binding of selected sdAbs presented in Example 1 against yeast surface displayed various hBCMA ecto-domain fragments.

FIG. 10 depicts the results of CAR-Jurkat assay wherein Jurkat cells were electroporated with varying CAR plasmids and CAR-J cells (Jurkat cells transiently expressing the CAR) cultured alone or in co-culture with BCMA-positive (Ramos or Jeko-1) or BCMA-negative (SKOV3) cell lines.

FIG. 11 depicts the results of CAR-T tonic activation assay wherein primary donor blood derived T cells were transduced with varying CAR constructs and examined for target-independent expansion.

FIG. 12 depicts the results of CAR-T target growth repression assay performed using donor blood derived T cells transduced with varying BCMA-single domain antibody or CD22-specific comparator CAR constructs.

FIG. 13 depicts the results of CAR-T target-specific activation assay performed using donor blood derived T cells transduced with varying BCMA-single domain antibody or CD22-specific comparator CAR constructs. Mock refers to unmodified donor derived T cells without CAR expression exposed to similar treatment conditions.

FIG. 14 depicts the results of CAR-T target-specific serial killing assay performed using long-term co-culture assay of donor blood derived CAR-T cells transduced with varying BCMA-single domain antibody or comparator CD22-CAR constructs.

FIG. 15 depicts the results of CAR-T co-culture assay performed over week 7 of the long-term co-culture assay. Donor blood derived CAR-T cells transduced with varying BCMA-single domain antibody, comparator CD22-CAR constructs, or with no CAR construct (mock).

FIG. 16 depicts the results of CAR-T co-culture assay performed over week 7 of the long-term co-culture assay. Donor blood derived CAR-T cells transduced with varying BCMA-single domain antibody, comparator CD22-CAR constructs, or with no CAR construct (mock).

FIG. 17A depicts partial results of consistency analysis and comparison with un-transduced T cells (mock) and BCMA-sdAb targeted CAR-transduced T cells generated from 2 separate donors. Additional data is presented in FIG. 17B. Graphs depict the total red fluorescent protein (NucLight) signal from marked target cells

FIG. 17B is a continuation of FIG. 17A, and depicts further results (red fluorescent protein (NucLight) signal) from the same set of experiments.

FIG. 18A depicts further data to FIGS. 17A and 17B, and specifically shows total green fluorescent protein signal from CAR cells as determined using automated counting.

FIG. 18B is a continuation of FIG. 18A, and depicts further results (green fluorescent protein signal) from the same experiments.

FIG. 19 depicts the results of an assay to test the activity of varying BCMA-specific CAR expressed within NK-cells.

FIG. 20 depicts the molecular structure of exemplary single domain antibody-based single-binder (left) or multi-binder (right) chimeric antigen receptor.

FIG. 21 depicts the results of Jurkat cell CAR activation activity assay wherein CAR plasmids with varying single or multi-binder formats were electroporated into Jurkat cells, which were then placed in co-cultures containing BCMA-positive Ramos cells (panel A), without target cells or with BCMA-negative (SKOV3) target cells (panel B).

FIG. 22 depicts result of tumor burden in mice that were inoculated with Ramos-FLUC and treated with various CAR-T cells

FIG. 23 depicts the proportion of surviving animals in each treatment group throughout the course of the experiment.

FIG. 24 depicts the molecular structure of BCMA-specific single domain antibody bi-specific T cell engager proteins with or without the inclusion of an additional hinge/spacer domain.

FIG. 25 depicts the results of Jurkat cell bi-specific T cell engager activation activity assay wherein HEK293T supernatants containing various bi-specific T cell engager molecules was placed on top of co-cultures containing Jurkat cells and BCMA-positive (Ramos) or BCMA-negative (U87vIII) target cells

FIG. 26 depicts the results of a bi-specific T cell engager activity assay using the same b-specific T cell engager containing HEK293T cell supernatants as described in FIG. 25; but using primary human T cells in co-culture with BCMA-positive target cells (Ramos).

### DETAILED DESCRIPTION

Generally, the present disclosure provides anti-BCMA single domain antibodies (sdAb) prepared by immunizing a llama with the ecto-domain of human B-cell maturation antigen (BCMA) that is preferentially expressed by mature B lymphocytes. By constructing a library of the heavy chain repertoire generated, VHH antibodies specific to the immunogen were isolated. The 13 unique example antibodies initially produced comprise CDR1, CDR2, and CDR3 sequences corresponding, respectively to SEQ NOs: 1-3, 4-6, 7-9, 10-12, 13-15, 16-18, 19-21, 22-24, 25-27, 28-30, 31-33, 34-36, 37-39; and related sequences. Also provided recombinant polypeptides comprising one or more of the sdAbs as herein defined. For example, multivalent antibodies are provided comprising any one of the sdAbs, including bispecific T-cell engagers, bispecific killer cell engagers (BiKEs), and trispecific killer cell engagers (TriKEs). Also described are chimeric antigen receptors (CARs) for CAR-T therapy comprising any one or more of the aforementioned sdAbs. Uses of these molecules in the treatment of cancer or autoimmune diseases are also described, in particular hematological malignancies such as multiple myeloma.

### Single Domain Antibodies & Polypeptides Comprising Them

A single domain antibody (sdAb), also known as a nanobody, is an antibody fragment consisting of a single monomeric variable antibody domain. sdAbs have been derived from heavy-chain antibodies found in Camelidae species (such as camel, llama, dromedary, alpaca and guanaco) using molecular biology techniques, which are also known as V_{H}H fragments (herein also termed “V_{H}H” or “VHH”). Other examples include V_{NAR} fragments derived from heavy chain antibodies found in cartilaginous fish, such as sharks. sdAbs have also been generated from a heavy chain/light chain of conventional immunoglobulin G (IgGs) by engineering techniques.

V_{H}H molecules are about 10 times smaller than IgG molecules. These single polypeptides are generally quite stable, often resisting extreme pH and temperature conditions that can be problematic for conventional antibodies and antibody fragments. Moreover, V_{H}Hs tend to be more resistant to the action of proteases. Furthermore, in vitro expression of V_{H}Hs tends to produce high yield of properly folded/functional VHHs. In addition, heavy chain antibodies and their engineered fragments (i.e., VHHs) generated in Camelidae species may recognize cryptic or hidden epitopes which otherwise inaccessible to larger conventional antibodies and antibody fragments generated in vitro through the use of antibody libraries or by immunization of other mammals.

In one aspect, there is provided an isolated single domain antibody (sdAb), which binds specifically to human BCMA, the sdAb comprising:
a) a CDR1 amino acid sequence GX₁X₂X₃X₄YX₅FV (SEQ ID NO: 59), wherein:
   X₁ is R or H,
   X₂ is A or T,
   X₃ is T or S,
   X₄ is D, N, or K, and
   X₅ is H, N, or Q,
a CDR2 amino acid sequence RX₆WX₇GX₈X₉P (SEQ ID NO: 60), wherein:
   X₆ is V or F,
   X₇ is S or G,
   X₈ is G or S, and
   X₉ is S or T, and
a CDR3 amino acid sequence AATKDIX₁₀SRX₁₁YX₁₂Y (SEQ ID NO: 61), wherein:
   X₁₀ is M or L,
   X₁₁ is S or G, and
   X₁₂ is D or V;
b) a CDR1 amino acid sequence GX₁X₂FGX₃X₄X₅ (SEQ ID NO: 62), wherein:
   X₁ is S or D,
   X₂ is I, S, or G,
   X₃ is T or A,
   X₄ is Y or H, and
   X₅ is N, A, or V,
a CDR2 amino acid sequence ISSAGX₆T (SEQ ID NO: 63), wherein:
   X₆ is N or S, and
a CDR3 amino acid sequence NGAPWADX₇X₈VKVX₉N (SEQ ID NO: 64), wherein:
   X₇ is A or E,
   X₈ is E or P, and
   X₉ is Y or W;
c) a CDR1 amino acid sequence GX₁X₂X₃X₄X₅X₆X₇ (SEQ ID NO: 65), wherein:
   X₁ is N, S, or D,
   X₂ is S, I, or P,
   X₃ is F or I,
   X₄ is G, D, or T,
   X₅ is A, V, or T,
   X₆ is Y or A, and
   X₇ is N or T,
a CDR2 amino acid sequence ISSX₈GX₉T (SEQ ID NO: 66), wherein:
   X₈ is T or A, and
   X₉ is N, T, or S, and
a CDR3 amino acid sequence NGAPWGDX₁₀X₁₁VKVX₁₂X₁₃ SEQ ID NO: 67), wherein:
   X₁₀ is D or A,
   X₁₁ is P or L,
   X₁₂ is W or E, and
   X₁₃ is S, D, T, or N;
d) a CDR1 amino acid sequence as set forth in SEQ ID NO: 31,
a CDR2 amino acid sequence as set forth in SEQ ID NO: 32, and
a CDR3 amino acid sequence as set forth in SEQ ID NO: 33 (from hBCMA VcMRo8 (VF7/VF8));
or
e) a CDR1 amino acid sequence as set forth in SEQ ID NO: 34,
a CDR2 amino acid sequence as set forth in SEQ ID NO: 35, and
a CDR3 amino acid sequence as set forth in SEQ ID NO: 36 (from hBCMA-A3).

In the above:
group a) provides consensus sequences defined by antibodies herein termed E7, H2, V3, 2F10, and 3F2,
group b) provides consensus sequences defined by antibodies herein termed B5, H4, and H1, and
group c) provides consensus sequences defined by antibodies herein termed F2, A6, V1/V6, and D2.

“CDRs” or “complementarity-determining regions” are the portion of the variable chains in immunoglobulins that collectively constitute the paratope, and thereby impart binding specificity and affinity to the antibody. As used here, the term refers to CDRs mapped in sdAbs according to the standards or conventions set by IMGT™ (international ImMunoGeneTics information system).

The antibodies described herein have been raised to the recombinant extracellular domain (ECD) of human BCMA isoform 1. An example mRNA sequence for this isoform may be found in GenBank entry BAB60895 wherein amino acids 1 to 54 correspond to the ECD (see also UniProt entry Q02223, and amino acids 1 to 54 thereof).

In one aspect, there is provided an isolated single domain antibody (sdAb), which binds specifically to human BCMA, the sdAb comprising:

A)
a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7 or hBCMA-2C3),
a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2 or hBCMA-4D1),
a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3),
a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5),
a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4),
a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1),
a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2),
a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6),
a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)),
a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2),
a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)),
a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10), or
a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2).

In one embodiment, the antibody comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7 or hBCMA-2C3).

In one embodiment, the antibody comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2 or hBCMA-4D1).

In one embodiment, the antibody comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3).

In one embodiment, the antibody comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5).

In one embodiment, the antibody comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4).

In one embodiment, the antibody comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1).

In one embodiment, the antibody comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2).

In one embodiment, the antibody comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6).

In one embodiment, the antibody comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)).

In one embodiment, the antibody comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2).

In one embodiment, the antibody comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)).

In one embodiment, the antibody comprises a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10).

In one embodiment, the antibody comprises a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2).

In one aspect, there is provided an isolated single domain antibody (sdAb), which binds specifically to human BCMA, the sdAb comprising:

A)
a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7 or hBCMA-2C3),
a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2 or hBCMA-4D1),
a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3),
a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5),
a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4),
a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1),
a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2),
a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6),
a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)),
a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2)
a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)),
a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10), or
a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2); or

B)

CDR1, CDR2, and CDR3 amino acid sequences that are at least 80% identical to the CDR1, CDR2, and CDR3 sequences defined in any one of part A) i) to xxviii).

In one embodiment, in B) the CDR 1 CDR2, and CDR3 amino acid sequences are at least 90% identical to the CDR1, CDR2, and CDR3 sequences defined in any one of part A) i) to xxviii). In one embodiment, in B) the CDR 1 CDR2, and CDR3 amino acid sequences are at least 95% identical to the CDR1, CDR2, and CDR3 sequences defined in any one of part A) i) to xxviii). In one embodiment, in B) the CDR 1 CDR2, and CDR3 amino acid sequences have at most three substitutions compared to the CDR1, CDR2, and CDR3 sequences defined in any one of part A) i) to xxviii). In one embodiment, in B) the CDR 1 CDR2, and CDR3 amino acid sequences have at most two substitutions compared to the CDR1, CDR2, and CDR3 sequences defined in any one of part A) i) to xxviii). In one embodiment, in B) the CDR 1 CDR2, and CDR3 amino acid sequences have at most one substitution compared to the CDR1, CDR2, and CDR3 sequences defined in any one of part A) i) to xxviii). In some embodiment, sequence differences vs. the sequences set forth in A) are conservative sequence substitutions.

The term “conservative amino acid substitutions” which is known in the art is defined herein as follows, with conservative substitutable candidate amino acids showing in parentheses: Ala (Gly, Ser); Arg (Gly, Gln); Asn (Gln; His); Asp (Glu); Cys (Ser); Gln (Asn, Lys); Glu (Asp); Gly (Ala, Pro); His (Asn; Gln); Ile (Leu; Val); Leu (Ile; Val); Lys (Arg; Gln); Met (Leu, Ile); Phe (Met, Leu, Tyr); Ser (Thr; Gly); Thr (Ser; Val); Trp (Tyr); Tyr (Trp; Phe); Val (Ile; Leu).

Sequence variants according to certain embodiments are intended to encompass molecules in which binding affinity and/or specificity is substantially unaltered vs. the parent molecule from which it is derived. Such parameters can be readily tested, e.g., using techniques described herein and techniques known in the art. Such embodiments may encompass sequence substitutions, insertions, or deletions.

In one aspect, there is provided an isolated single domain antibody (sdAb), which binds specifically to human BCMA, the sdAb comprising:
a CDR3 amino acid sequence as set forth in SEQ ID NO: 3,
a CDR3 amino acid sequence as set forth in SEQ ID NO: 6,
a CDR3 amino acid sequence as set forth in SEQ ID NO: 9,
a CDR3 amino acid sequence as set forth in SEQ ID NO: 12,
a CDR3 amino acid sequence as set forth in SEQ ID NO: 15,
a CDR3 amino acid sequence as set forth in SEQ ID NO: 18,
a CDR3 amino acid sequence as set forth in SEQ ID NO: 21,
a CDR3 amino acid sequence as set forth in SEQ ID NO: 24,
a CDR3 amino acid sequence as set forth in SEQ ID NO: 27,
a CDR3 amino acid sequence as set forth in SEQ ID NO: 30,
a CDR3 amino acid sequence as set forth in SEQ ID NO: 33,
a CDR3 amino acid sequence as set forth in SEQ ID NO: 36, or
a CDR3 amino acid sequence as set forth in SEQ ID NO: 39.

Recognizing that CDR3 is often the major determinant of binding for V_{H}H sdAbs, it would be understood that other CDRs could be mutagenized or otherwise diversified and a resulting library (or candidate molecule) screened for antibodies that bind to BCMA and/or cross-compete for binding to BCMA with the parent molecule. These embodiments are intended to cover, inter alia, molecules identified in this manner.

In one embodiment, the isolated single domain antibody (sdAb) of claim 4, comprises:
a CDR1 amino acid sequence as set forth in SEQ ID NO: 1, and a CDR3 amino acid sequence as set forth in SEQ ID NO: 3,
a CDR1 amino acid sequence as set forth in SEQ ID NO: 4, and a CDR3 amino acid sequence as set forth in SEQ ID NO: 6,
a CDR1 amino acid sequence as set forth in SEQ ID NO: 7, and a CDR3 amino acid sequence as set forth in SEQ ID NO: 9,
a CDR1 amino acid sequence as set forth in SEQ ID NO: 10, and a CDR3 amino acid sequence as set forth in SEQ ID NO: 12,
a CDR1 amino acid sequence as set forth in SEQ ID NO: 13, and a CDR3 amino acid sequence as set forth in SEQ ID NO: 15,
a CDR1 amino acid sequence as set forth in SEQ ID NO: 16, and a CDR3 amino acid sequence as set forth in SEQ ID NO: 18,
a CDR1 amino acid sequence as set forth in SEQ ID NO: 19, and a CDR3 amino acid sequence as set forth in SEQ ID NO: 21,
a CDR1 amino acid sequence as set forth in SEQ ID NO: 22, and a CDR3 amino acid sequence as set forth in SEQ ID NO: 24,
a CDR1 amino acid sequence as set forth in SEQ ID NO: 25, and a CDR3 amino acid sequence as set forth in SEQ ID NO: 27,
a CDR1 amino acid sequence as set forth in SEQ ID NO: 28, and a CDR3 amino acid sequence as set forth in SEQ ID NO: 30,
a CDR1 amino acid sequence as set forth in SEQ ID NO: 31, and a CDR3 amino acid sequence as set forth in SEQ ID NO: 33,
a CDR1 amino acid sequence as set forth in SEQ ID NO: 34, and a CDR3 amino acid sequence as set forth in SEQ ID NO: 36, or
a CDR1 amino acid sequence as set forth in SEQ ID NO: 37, and a CDR3 amino acid sequence as set forth in SEQ ID NO: 39.

These embodiments are intended to encompass, inter alia, embodiments in which molecules recovered following mutagenization/diversification of CDR2, and screening for variant molecules that bind to BCMA and/or cross-compete for binding to BCMA with the parent molecule from which they are defined. As above, a library could be screened or individual candidate molecules could be tested.

In one embodiment, sdAb comprises A) the amino acid sequence of any one of SEQ ID NO: 40 to 58, 79, and 80, or B) an amino acid sequence that is at least 80% identical to any one of SEQ ID NO: 40 to 58, 79, and 80 across the full length thereof. In one embodiment, the amino acid sequence of B) is at least 85% identical across the full length therefore to one of the amino acid sequences of A). In one embodiment, the amino acid sequence of B) is at least 90% identical across the full length therefore to one of the amino acid sequences of A). In one embodiment, the amino acid sequence of B) is at least 95% identical across the full length therefore to one of the amino acid sequences of A). In one embodiment, the amino acid sequence of B) is at least 98% identical across the full length therefore to one of the amino acid sequences of A). In one embodiment, the amino acid sequence of B) is at least 98% identical across the full length therefore to one of the amino acid sequences of A). In some of these embodiments, sequences differences vs. sequences of A) are outside the CDR sequences.

In one embodiment, the sdAb comprises A) the amino acid sequence of any one of SEQ ID NO: 40 to 58, 79, and 80.

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment, the sdAb comprises a CDR1, CDR2, and CDR3 of the sdAb sequence set forth in SEQ ID NO: 49.

In one embodiment, the sdAb comprises a CDR1, CDR2, and CDR3 of the sdAb sequence set forth in SEQ ID NO: 50.

In one embodiment, the sdAb comprises a CDR1, CDR2, and CDR3 of the sdAb sequence set forth in SEQ ID NO: 51.

In one embodiment, the sdAb comprises a CDR1, CDR2, and CDR3 of the sdAb sequence set forth in SEQ ID NO: 52.

In one embodiment, the sdAb comprises a CDR1, CDR2, and CDR3 of the sdAb sequence set forth in SEQ ID NO: 53.

In one embodiment, the sdAb comprises a CDR1, CDR2, and CDR3 of the sdAb sequence set forth in SEQ ID NO: 54.

In one embodiment, the sdAb comprises a CDR1, CDR2, and CDR3 of the sdAb sequence set forth in SEQ ID NO: 55.

In one embodiment, the sdAb comprises a CDR1, CDR2, and CDR3 of the sdAb sequence set forth in SEQ ID NO: 56.

In one embodiment, the sdAb comprises a CDR1, CDR2, and CDR3 of the sdAb sequence set forth in SEQ ID NO: 57.

In one embodiment, the sdAb comprises a CDR1, CDR2, and CDR3 of the sdAb sequence set forth in SEQ ID NO: 58.

In one embodiment, the sdAb comprises a CDR1, CDR2, and CDR3 of the sdAb sequence set forth in SEQ ID NO: 79.

In one embodiment, the sdAb comprises a CDR1, CDR2, and CDR3 of the sdAb sequence set forth in SEQ ID NO: 80.

In one embodiment of the above, CDR1, CDR2, and CDR3 are defined with respect to the IMGT numbering system. It is to be appreciated that CDR sequences could be defined by other conventions, such as the Kabat, Chothia, or EU numbering systems.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In one embodiment, the sdAb comprises SEQ ID NO: 49.

In one embodiment, the sdAb comprises SEQ ID NO: 50.

In one embodiment, the sdAb comprises SEQ ID NO: 51.

In one embodiment, the sdAb comprises SEQ ID NO: 52.

In one embodiment, the sdAb comprises SEQ ID NO: 53.

In one embodiment, the sdAb comprises SEQ ID NO: 54.

In one embodiment, the sdAb comprises SEQ ID NO: 55.

In one embodiment, the sdAb comprises SEQ ID NO: 56.

In one embodiment, the sdAb comprises SEQ ID NO: 57.

In one embodiment, the sdAb comprises SEQ ID NO: 58.

In one embodiment, the sdAb comprises SEQ ID NO: 79.

In one embodiment, the sdAb comprises SEQ ID NO: 80.

In one embodiment, the sdAb is a Camelidae V_{H}H sdAb.

In one embodiment, the sdAb is a llama V_{H}H sdAb

In one embodiment, the sdAb is humanized camelidae V_{H}H.

By the term “humanized” as used herein is meant mutated so that immunogenicity upon administration in human patients is minor or nonexistent. Humanizing a polypeptide, according to the present invention, comprises a step of replacing one or more of the Camelidae amino acids by their human counterpart as found in the human consensus sequence, without that polypeptide losing its typical character, i.e. the humanization does not significantly affect the antigen binding capacity of the resulting polypeptide. A humanized antibody can be produced using a variety of techniques known in the art, including but not limited to, CDR-grafting, veneering or resurfacing, chain shuffling, etc.

In one embodiment, the isolated sdAb binds to an epitope in a portion of BCMA from Gly6 to Pro23. In one embodiment, the sdAb binding to this epitope is hBCMA-E7, hBCMA-H2, or hBCMA-V3 as defined herein. In one embodiment, the sdAb binding to this epitope comprises the CDRs of hBCMA-E7, hBCMA-H2, or hBCMA-V3 as defined herein.

In one embodiment, the isolated sdAb binds to an epitope in a portion of BCMA from Gly6 to Tyr40. In one embodiment, the sdAb binding to this epitope is hBCMA-A6, hBCMA-H4, or hBCMA VcMRo8 (VF7/VF8) as defined herein. In one embodiment, the sdAb binding to this epitope comprises the CDRs of hBCMA-A6, hBCMA-H4, or hBCMA VcMRo8 (VF7/VF8) as defined herein.

In one embodiment, the sdAb has an affinity for human BCMA of 2.5×10⁻⁷ nM or less. In one embodiment, the sdAb has an affinity for human BCMA of 3×10⁻⁸ nM or less. In one embodiment, the sdAb has an affinity for human BCMA of 9.6×10⁻⁹ nM or less. In one embodiment, the sdAb has an affinity for human BCMA of 9.3×10⁻¹⁰ nM or less. In one embodiment, the sdAb has an affinity for human BCMA of 7×10⁻¹² nM or less. Binding affinity can be determined, e.g., according to assays described herein.

In one aspect, there is provided a VHH single domain antibody (sdAb) that competes for specific binding to BCMA with one of the isolated sdAbs described above (a “competing sdAb”). A competing sdAb may be identified by a method that comprises a binding assay which assesses whether or not a test antibody is able to cross-compete with a known antibody of the invention for a binding site on the target molecule. For example, the antibodies described hereinabove may be used as reference antibodies. Methods for carrying out competitive binding assays are well known in the art. For example they may involve contacting together a known antibody of the invention and a target molecule under conditions under which the antibody can bind to the target molecule. The antibody/target complex may then be contacted with a test antibody and the extent to which the test antibody is able to displace the antibody of the invention from antibody/target complexes may be assessed. An alternative method may involve contacting a test antibody with a target molecule under conditions that allow for antibody binding, then adding an antibody of the invention that is capable of binding that target molecule and assessing the extent to which the antibody of the invention is able to displace the test antibody from antibody/target complexes. Such antibodies may be identified by generating new sdAbs to BCMA and screening the resulting library for cross-competition. Alternatively, one of the antibodies described herein may serve as a starting point for diversification, library generation, and screening. A further alternative could involve testing individual variants of an antibody described herein.

In one embodiment, the sdAb defined herein is a camelid sdAb.

In one embodiment, the sdAb defined herein is a llama sdAb.

In one embodiment, the sdAb defined herein is humanized form of camelidae sdAb.

Table 1 lists full-length sequences for various sdAb disclosed herein according to some embodiments. CDR1, CDR2, and CDR3 sequences are underlined. CDR identification and numbering used herein is according to the IMGT™ convention.

TABLE 1
VHH Sequences

| Name | Sequence (CDRs Underlined) |
|---|---|
| hBCMA-E7 (E7) | QVQLVESGGGLVQTGDSLRLACTISGRATDHFVMAWERRAPGKEREYVAT |
| | RVWSGGSPYYLDSVKGRFAIAIDNAKNTAYLQMNNLKPEDTAVYYCAATK |
| | DIMSRSYDYWGLGTQVTVSS |
| hBCMA-2C3 | QVKLEESGGGLVQTGDSLRLACTISGRATDHFVMAWERRAPGKEREYVAT |
| (variant of E7 | RVWSGGSPYYLDSAKGRFAIAIDNAKNTAYLQMNNLKPEDTAVYYCAATK |
| with the same | DIMSRSYDYWGLGTQVTVSS |
| CDRs) | |
| hBCMA-H2 (H2) | QVKAEESGGGLVRPGDSLRLTCTISGRTSNNFVMAWERRTPGKEREYVAT |
| | RVWSGSTPYYHDSVKGRFTISIDDDKNTAYLQMNSLKPEDTAVYYCAATK |
| | DIMSRSYDYWGLGTQVTVSS |
| hBCMA-4D1 | QVQLVESGGGLVRPGDSLRLTCTISGRTSNNFVMAWERRTPGKEREYVAT |
| (variant of H2 | RVWSGSTPYYHDSVKGRFTISIDDDKNTAYLQMNSLKPEDTAVYYCAATK |
| with the same | DIMSRSYDYWGLGTQVTVSS |
| CDRs) | |
| hBCMA-VcMRo3 | EVQLEQSGGGLVQTGDSLRLTCTIPGRASNHFVMAWERRAPGKEREFVAT |
| (V3) | RVWSGGSPYYSDSVKGRFAIAIDNAKNTAYLQMNSLKPEDTAVYYCAATK |
| | DIMSRSYDYWGLGTQVTVSS |
| hBCMA-2F10 | QVQLVESGGGLVQSGNSLRLSCSISGHTTKNFVMAWERRAPGKERAIVAT |
| (2F10) | RVWSGGSPWYSESAKGRFTISIDDARNTAYLQMNNLKPEDTAVYYCAATK |
| | DIMSRGYVYWGLGTQVTVSS |
| hBCMA-3F2 (3F2) | QVQLVESGGRLVQTGDSLRLTCEISGHTSNQFVLAWFRRPPGKEREVVAT |
| | RFWGGGSPYYSDSVRGRFAIAIDDAKNTAYLQMSSLKPEDTAVYYCAATK |
| | DILSRGYDYWGQGTQVTVSS |
| hBCMA-B5 (B5) | QVKLEESGGGLVQPGGSLRLSCAASGSIFGTYNMGWYRQAPGKQRELVAA |
| | ISSAGNTFYRDSVKGRFTVSRDNAKNTVYLQMDRLKYEDTAVYNCNGAPW |
| | ADAEVKVYNWGQGTQVTVSS |
| hBCMA-H4 (H4) | QVQLVESGGGLVQPGGSLRLSCAASGDSFGAYAMGWYRQAPGKQRELVAA |
| | ISSAGNTFYRDSVKGRFTVSRNNAKNAMYLQMDRLKPEDTAVYQCNGAPW |
| | ADEPVKVWNWGLGTQVTVSS |
| hBCMA-H1 (H1) | QVQLVESGGGLVQPGGSLRLSCAASGSGFGTHVMGWYRQAPGKPRELVAA |
| | ISSAGSTFYRDSVKGRFTVSRDNAKNTMYLQMDRLKPEDTAVYYCNGAPW |
| | ADEPVKVWNWGQGTQVTVSS |
| hBCMA-F2 (F2) | QVKLEESGGGLVKPGGSLRLSCGASGNSFGAYNMGWYRQAPGKQRELVAA |
| | ISSTGNTFYRDSVRGRFTVSRDNAKSTMSLQMERLKPEDTAVYLCNGAPW |
| | GDDPVKVWSWGQGTQVTVSS |
| hBCMA-A6 (A6) | QVQLVESGGGLVQPGGSLRLSCVASGSIFDAYNMGWYRQAPGKQRELVAA |
| | ISSAGTTFYRDSVKGRFTVSRNNAKNTMYLQMDRLRPEDTAVYDCNGAPW |
| | GDAPVKVEDWGQGTQVTVSS |
| hBCMA-VcMRo1 | EVQLQQSGGGLVQAGESLTISCAVEGDPITVATMGWYRQAPGKLRELVAA |
| (V1/V6) | ISSAGSTFYRDSVRGRFTVSRDNAKSTMYLQMDRLKVEDTAVYSCNGAPW |
| | GDAPVKVWTWGEGTQVTVSS |
| hBCMA-D2 (D2) | QAQVQLVESGGGLVQPGGSLRLSCAASGSIFGTYNMGWYRQAPGKQRELV |
| | AAISSAGNTFYRDSVKGRFTVSRDNAKNTMYLQMDRLKYEDTAVYNCNGA |
| | PWGDALVKVWNWGQGTQVTVSS |
| hBCMA-VMRo8 | QVQLVESGGGLVQPGGSLRLSCAASGTIFSRNALAWFRQAPGKQRELVAH |
| (VF7/VF8) (V8) | ITIGGTTVYKDSVKGRFTISRDNAKNTVYLQMDALKPEDTAVYYCNADPE |
| | GSWNWVRRGDYWGQGTQVTVS |

Table 2 provides correspondence between abbreviated antibody names used herein, and SEQ ID NOs for CDR1, CDR2, CDR3, and full-length sequences for each sdAb.

TABLE 2
VHH Sequence ID Numbers

Sequence ID Nos.

| Name | CDR1 | CDR2 | CDR3 | Full Length |
|---|---|---|---|---|
| E7 | 1 | 2 | 3 | 40 |
| H2 | 4 | 5 | 6 | 41 |
| V3 | 7 | 8 | 9 | 42 |
| B5 | 10 | 11 | 12 | 43 |
| H4 | 13 | 14 | 15 | 44 |
| H1 | 16 | 17 | 18 | 45 |
| F2 | 19 | 20 | 21 | 46 |
| A6 | 22 | 23 | 24 | 47 |
| V1/V6 | 25 | 26 | 27 | 48 |
| D2 | 28 | 29 | 30 | 49 |
| V8 | 31 | 32 | 33 | 50 |
| 2F10 | 34 | 35 | 36 | 51 |
| 3F2 | 37 | 38 | 39 | 52 |
| 2C3 | 1 | 2 | 3 | 79 |
| 4D1 | 4 | 5 | 6 | 80 |

Table 4 (see below) provides additional alternative sequences of certain sdAbs used in constructs according to some embodiments (see, e.g., SEQ ID NOs: 53 to 58). These sequences encompass, in some cases, modifications of the N-terminal region. These modifications may result in increased stability and/or affinity. It is also noted that certain of these sequences also contain sequences differences in framework regions that arose during cloning. These sequence differences are encompassed according to some embodiments (see, e.g., the third position of FR4).

In one aspect, there is provided a VHH single domain antibody (sdAb) that competes for specific binding to BCMA with one of the isolated sdAbs described above.

### Recombinant Polypeptides

In one aspect, there is provided a recombinant polypeptide comprising one or more sdAb as defined herein. In one embodiment, there is provided a recombinant polypeptide comprising two or more sdAb as defined herein. In one embodiment, there is provided a recombinant polypeptide comprising two or more sdAb as defined herein.

### V_{H}H:Fc Fusions

In one aspect, there is provided the sdAb defined herein fused to a human Fc (termed a “V_{H}H:Fc fusion”). For example, the V_{H}H:Fc fusion may comprise at least a CH2 and a CH3 of the IgG, IgA, or IgD isotype. The V_{H}H:Fc fusion may comprise at least a CH2, a CH3, and a CH4 of the IgM or IgE isotype. Such embodiments may be useful in activating the immune system in higher order recombinant molecules. For example, according to some embodiments, two such Fc-containing V_{H}H:Fc fusions may assemble to form a recombinant monomeric antibody. In some embodiment, such a monomeric antibody is capable of activating the immune system. Such monomeric antibodies may be of IgG, IgA, IgD, IgE, or IgM isotype. In one embodiment, IgA Fc-containing V_{H}H:Fc fusions may also assemble into a recombinant dimeric (secretory) form. Multimeric forms are also envisaged in some embodiments. For example, five IgM monomers may assemble to form a recombinant pentameric antibody.

In some embodiments, the multivalent antibody described herein may be an assembly of the same VHH:Fc fusions.

In some embodiments, the multivalent antibody described herein may be an assembly of the different VHH:Fc fusions having the same binding target. For example, these may bind to different epitopes on the same target molecule. Examples may include assemblies of different VHH:Fc fusions, each comprising a different anti-BCMA sdAb as defined herein.

In some embodiments, the multivalent antibody described herein may be an assembly of an VHH:Fc fusion defined herein (comprising an anti-BCMA sdAb as defined herein) and another VHH:Fc fusion comprising a paratope directed to a different target.

### Fusions to Cargo Molecules

In a further aspect, the present disclosure provides anti-BCMA sdAb as defined herein linked to a cargo molecule. The cargo molecule may comprise, for example, a therapeutic moiety, such as for example, a cytotoxic agent, a cytostatic agent, an anti-cancer agent or a radiotherapeutic. In particular embodiments of the disclosure, the antibody drug conjugates may comprise a cytotoxic agent. Another particular embodiment of the disclosure relates to antibody drug conjugates comprising a radiotherapeutic.

### Nucleic Acid Molecules

In aspect, there is provided a nucleic acid molecule encoding an sdAb, the recombinant polypeptide, or the V_{H}H:Fc fusion as defined herein. In one embodiment, the nucleic acid molecule may comprise DNA. In one embodiment, the nucleic acid molecule may comprise RNA. In one embodiment, the nucleic acid molecule may comprise mRNA. In one embodiment, the nucleic acid molecule may comprise any nucleic acids that encode a protein. In one embodiment, nucleic acid molecule is a vector.

### Compositions

In one aspect, there is provided a composition comprising an sdAb as defined herein, or a polypeptide comprising such an sdAb; together with an acceptable excipient, diluent or carrier. In one embodiment the composition is a pharmaceutical composition, and the excipient, diluent or carrier is a pharmaceutically acceptable excipient, diluent or carrier.

### Uses & Methods

In one aspect, there is provided a use of the sdAb as defined herein or of an antibody comprising one or more V_{H}H:Fc fusion as defined herein for treatment of a cancer or an auto-immune disease. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect, there is provided a use of the sdAb as defined herein or of an antibody comprising one or more V_{H}H:Fc fusion as defined herein for preparation of a medicament for treatment of a cancer or an auto-immune disease. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect, there is provided the sdAb as defined herein or of an antibody comprising one or more V_{H}H:Fc fusion as defined herein for use in treatment of a cancer or an auto-immune disease. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect, there is provided a method of treating a cancer or an auto-immune disease in subject comprising administering to the subject the sdAb as defined herein or of an antibody comprising one or more V_{H}H:Fc fusion as defined herein. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

### Multivalent Antibodies & Related Embodiments

In one aspect, there is provided a multivalent antibody comprising an sdAb as defined above.

By “multivalent antibody” is use herein to mean a molecule comprising more than one variable region or paratope for binding to one or more antigen(s) within the same or different target molecule(s).

In some embodiments, the paratopes may bind to different epitopes on the same target molecule. In some embodiments, the paratopes may bind to different target molecules. In these embodiments, the multivalent antibody may be termed bispecific, trispecific, or multispecific, depending on the number of paratopes of different specificity that are present. As the multivalent antibody comprises one of the anti-BCMA sdAbs as herein defined, the multivalent antibody comprises BCMA binding affinity.

For example, as explained above, in some embodiments a multivalent antibody may be an assembly of a V_{H}H:Fc fusion defined herein (comprising an sdAb as defined herein) and another V_{H}H:Fc fusion comprising a different paratope conferring a different specificity.

In one embodiment, there is provided a bispecific antibody comprising an sdAb as defined above, and a second antigen-binding portion. In some embodiments, the second antigen binding portion may comprise a monoclonal antibody, an Fab, and F(ab′)₂, an Fab′, an scFv, or an sdAb, such as a V_{H}H or a V_{NAR}.

An “antigen-binding portion” is meant a polypeptide that comprises an antibody or antigen-binding fragment thereof having antigen-binding activity, including engineered antibodies fragments thereof.

In some embodiments, the second antigen-binding portion may bind to human serum albumin, e.g., for the purposes of stabilization/half-life extension.

In one embodiment, there is provided a trispecific antibody comprising an sdAb as defined above, and a second-binding portion, and a third antigen-binding portion. In some embodiments, the second antigen binding portion comprises a monoclonal antibody, an Fab, and F(ab′)₂, and Fab′, an sdFv, or an sdAb, such as a V_{H}H or a V_{NAR}. In some embodiments, the third antigen binding portion comprises, independently, a monoclonal antibody, an Fab, and F(ab′)₂, and Fab′, an sdFv, or an sdAb, such as a V_{H}H or a V_{NAR}.

The second and/or third antigen-binding portion may bind to human serum albumin, e.g., for the purposes of stabilization/half-life extension.

In some embodiments, the trispecific antibody may be multispecific and the antibody may comprise one or more additional antigen-binding portion(s). In such embodiments, the additional antigen-binding portion(s) may be, independently, an Fab, and F(ab′)₂, and Fab′, an sdFv, or an sdAb, such as a V_{H}H or a V_{NAR}.

In one embodiment, the multispecific antibody comprises a first antigen-binding portion comprising an sdAb as defined herein, and a second antigen-binding portion. In one embodiment, the second antigen-binding moiety binds specifically to a cell-surface marker of an immune cell.

A “cell surface marker” is a molecule expressed at the surface of the cell that is particular to (or enriched in) a cell type, and that is capable of being bound or recognized by an antigen-binding portion.

### Bispecific T-Cell Engager

In one embodiment, the multivalent antibody is a bispecific T-cell engager comprising an sdAb as defined herein and second antigen-binding moiety that binds specifically to a cell-surface marker of a T-cell. In one embodiment, the T-cell marker comprises human CD3.

Human CD3, we will be recognized, is a multi-subunit antigen, of which various subunits may participate in CD3 activation. One such subunit is CD3 epsilon (see, e.g., GenBank NP_000724.1). Other non-limiting examples include CD3 gamma (see, e.g., GenBank NP_000064.1) and delta (see, e.g., GenBank NP_000723.1 for delta isoform A, and, e.g., GenBank NP_001035741.1 for delta isoform B).

In some embodiments, T-cell marker comprises CD3 epsilon, CD3 gamma, or CD3 delta. In one specific embodiment, the T-cell marker comprises CD3 epsilon.

The term “bispecific T-cell engager”, as used herein, refers to a recombinant bispecific protein that has two linked variable regions from two different antibodies, one targeting a cell-surface molecule on T cells (for example, CD3ε), and the other targeting antigens on the surface of disease cells, typically malignant cells. For example a bispecific T-cell engager may comprises an sdAb as defined herein and an scFvs. A bispecific T-cell engager may comprise an sdAb as defined herein and a second VHH/sdAb. The two variable regions are typically linked together by a short flexible linker such as GlySer linker. By binding to tumor antigens and T cells simultaneously, bispecific T-cell engagers mediate T-cell responses and killing of tumor cells. The T-cell/target cell adherence facilitated by a bispecific T-cell engager is independent of MHC haplotype.

In one embodiment, the bispecific T-cell engager comprises in N-terminal to C-terminal direction:
the first antigen-binding portion,
an amino acid linker, and
the second antigen-binding portion.

In one embodiment, the signal peptide further comprises a signal peptide N-terminal to the first antigen-binding portion.

A “signal peptide”, as referred to herein allows the nascent protein to be directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. The signal peptide may be at the amino terminus of the molecule.

In one embodiment, the signal peptide is a signal peptide from human CD28. In one embodiment, the signal peptide from human CD28 comprises SEQ ID NO: 69. In one embodiment, the signal peptide is at least 80% identical to SEQ ID NO: 69. In one embodiment, the signal peptide is at least 90% identical to SEQ ID NO: 69. In one embodiment, the signal peptide is at least 95% identical to SEQ ID NO: 69. In one embodiment, the signal peptide is at least 98% identical to SEQ ID NO: 69.

By “amino acid linker”, in this context, will be understood a sequence of sufficient length, flexibility, and composition to permit the bispecific T-cell engager to be properly functional an engage with both targets.

The amino acid linker may comprise a hinge. The hinge may be from human CD8, e.g. as set forth in SEQ ID NO: 71. The amino acid linker may, in some embodiments, comprises additional amino acids positioned N- and/or C-terminally with respect to the hinge. For example, the amino acid linker may comprise SEQ ID NO: 75 positioned N- and C-terminally with respect to SEQ ID NO: 71, SEQ ID NO: 70 positioned N- and C-terminally with respect to SEQ ID NO: 71, or a combination thereof. Fragments of SEQ ID NO: 70 of 5, 6, 7, 8, 9, 10, 11, 12, 13, 14, 15, 16, 17 amino acids (or combinations thereof) could also be positioned N- and/or C-terminally with respect to the hinge.

In one embodiment the amino acid linker comprises (N to C) SEQ ID NO: 70-SEQ ID NO: 71-SEQ ID NO: 75. In one embodiment the amino acid linker consists of (N to C) SEQ ID NO: 70-SEQ ID NO: 71-SEQ ID NO: 75.

In one embodiment, the multivalent antibody is encoded by SEQ ID NO: 76.

In one embodiment, the sdAb comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7 or hBCMA-2C3).

In one embodiment, the sdAb comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2 or hBCMA-4D1).

In one embodiment, the sdAb comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3).

In one embodiment, the sdAb comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5).

In one embodiment, the sdAb comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4).

In one embodiment, the sdAb comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1).

In one embodiment, the sdAb comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2).

In one embodiment, the sdAb comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6).

In one embodiment, the sdAb comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)).

In one embodiment, the sdAb comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2).

In one embodiment, the ant sdAb comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)).

In one embodiment, the sdAb comprises a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10).

In one embodiment, the sdAb comprises a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2).

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment, the sdAb comprises a CDR1, CDR2, and CDR3 of the sdAb sequence set forth in SEQ ID NO: 49.

In one embodiment, the sdAb comprises a CDR1, CDR2, and CDR3 of the sdAb sequence set forth in SEQ ID NO: 50.

In one embodiment, the sdAb comprises a CDR1, CDR2, and CDR3 of the sdAb sequence set forth in SEQ ID NO: 51.

In one embodiment, the sdAb comprises a CDR1, CDR2, and CDR3 of the sdAb sequence set forth in SEQ ID NO: 52.

In one embodiment, the sdAb comprises a CDR1, CDR2, and CDR3 of the sdAb sequence set forth in SEQ ID NO: 53.

In one embodiment, the sdAb comprises a CDR1, CDR2, and CDR3 of the sdAb sequence set forth in SEQ ID NO: 54.

In one embodiment, the sdAb comprises a CDR1, CDR2, and CDR3 of the sdAb sequence set forth in SEQ ID NO: 55.

In one embodiment, the sdAb comprises a CDR1, CDR2, and CDR3 of the sdAb sequence set forth in SEQ ID NO: 56.

In one embodiment, the sdAb comprises a CDR1, CDR2, and CDR3 of the sdAb sequence set forth in SEQ ID NO: 57.

In one embodiment, the sdAb comprises a CDR1, CDR2, and CDR3 of the sdAb sequence set forth in SEQ ID NO: 58.

In one embodiment, the sdAb comprises a CDR1, CDR2, and CDR3 of the sdAb sequence set forth in SEQ ID NO: 79.

In one embodiment, the sdAb comprises a CDR1, CDR2, and CDR3 of the sdAb sequence set forth in SEQ ID NO: 80.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In one embodiment, the sdAb comprises SEQ ID NO: 49.

In one embodiment, the sdAb comprises SEQ ID NO: 50.

In one embodiment, the sdAb comprises SEQ ID NO: 51.

In one embodiment, the sdAb comprises SEQ ID NO: 52.

In one embodiment, the sdAb comprises SEQ ID NO: 53.

In one embodiment, the sdAb comprises SEQ ID NO: 54.

In one embodiment, the sdAb comprises SEQ ID NO: 55.

In one embodiment, the sdAb comprises SEQ ID NO: 56.

In one embodiment, the sdAb comprises SEQ ID NO: 57.

In one embodiment, the sdAb comprises SEQ ID NO: 58.

In one embodiment, the sdAb comprises SEQ ID NO: 79.

In one embodiment, the sdAb comprises SEQ ID NO: 80.

In some embodiments, the bi-specific T-cell engager is a sequence variant of the above bi-specific T-cell engager having 80%, 90%, 95%, 98%, or 99% identity to one of the above-described bi-specific T-cell engagers. In some embodiments, the variant retains substantially the same binding specificity as the parent molecule from which it is derived. In some embodiments the variant retains substantially the same binding affinity as the parent molecule from which it is derived.

### BiKEs & TriKEs

In one embodiment, the multivalent antibody is a bispecific killer cell engager.

The term “BiKE” refers to a recombinant bispecific protein that has two linked variable regions from two different antibodies, one targeting a cell-surface molecule on natural killer (NK) cells (for example, CD16), and the other targeting antigens on the surface of disease cells, typically malignant cells. For example the BiKE may comprises two scFvs, two VHHs, or a combination thereof. The two are typically linked together by a short flexible linker. By binding to tumor antigens and NK cells simultaneously, BiKEs mediate NK-cell responses and killing of tumor cells.

In one embodiment, the cell-surface marker of the immune cell comprises a natural killer (NK) cell marker. In one embodiment, the NK cell marker comprises human CD16.

In one embodiment, the multivalent antibody is a trispecific killer cell engager (BiKE).

The term “TriKE” indicates at a BiKE that has been further modified to include another functionality. This term has been used to encompass various approaches. One approach involves inserting an intervening immunomodulatory molecule (a modified human IL-15 crosslinker) to promote NK cell activation, expansion, and/or survival (Vallera et al. IL-15 Trispecific Killer Engagers (TriKEs) Make Natural Killer Cells Specific to CD33+ Targets While Also Inducing In Vivo Expansion, and Enhanced Function. Clinical Cancer Research. 2012; 22(14): 3440-50). Other TriKE approaches are trispecific molecules that include three antibody variable regions: one targeting an NK cell receptor and two that target tumour-associated antigens (Gleason et al. Bispecific and Trispecific Killer Cell Engagers Directly Activate Human NK Cells Through CD16 Signaling and Induce Cytotoxicity and Cytokine Production. Mol Cancer Ther. 2012; 11(12): 2674-84). Yet other TriKE approaches target two NK cell receptors (e.g., CD16 and NKp46) and one tumour-associated antigen (Gauthier et al. Multifunctional Natural Killer Cell Engagers Targeting NKp46 Trigger Protective Tumor Immunity. Cell. 2019; 177(7): 1701-13).

In one embodiment, the multivalent antibody further comprises a cytokine for stimulating activation, expansion, and/or survival of NK cells. In one embodiment, the cytokine for stimulating expansion of NK cells is interleukin-15 (IL15), a variant thereof, or a functional fragment thereof.

In one embodiment, the multivalent antibody further comprises at least a third antigen-binding portion that binds to a second NK cell marker. In one embodiment, the second NK cell marker is human NKp46.

In one embodiment, the multivalent antibody further comprises at least a third antigen-binding portion that binds to a tumour-associated antigen. In some embodiment, the tumour-associated antigen is distinct from human BCMA.

In one embodiment, the third antigen-binding portion comprises a V_{H}H, a V_{NAR}, or an scVF.

In one embodiment, the second antigen-binding portion comprises a V_{H}H.

In one embodiment, the third antigen-binding portion binds to human serum albumin. In such embodiment, the affinity for human serum albumin may contribute to stabilization/increased half-life.

In one embodiment, the sdAb comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7 or hBCMA-2C3).

In one embodiment, the sdAb comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2 or hBCMA-4D1).

In one embodiment, the sdAb comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3).

In one embodiment, the sdAb comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5).

In one embodiment, the sdAb comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4).

In one embodiment, the sdAb comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1).

In one embodiment, the sdAb comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2).

In one embodiment, the sdAb comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6).

In one embodiment, the sdAb comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)).

In one embodiment, the sdAb comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2).

In one embodiment, the ant sdAb comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)).

In one embodiment, the sdAb comprises a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10).

In one embodiment, the sdAb comprises a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2).

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment, the sdAb comprises a CDR1, CDR2, and CDR3 of the sdAb sequence set forth in SEQ ID NO: 49.

In one embodiment, the sdAb comprises a CDR1, CDR2, and CDR3 of the sdAb sequence set forth in SEQ ID NO: 50.

In one embodiment, the sdAb comprises a CDR1, CDR2, and CDR3 of the sdAb sequence set forth in SEQ ID NO: 51.

In one embodiment, the sdAb comprises a CDR1, CDR2, and CDR3 of the sdAb sequence set forth in SEQ ID NO: 52.

In one embodiment, the sdAb comprises a CDR1, CDR2, and CDR3 of the sdAb sequence set forth in SEQ ID NO: 53.

In one embodiment, the sdAb comprises a CDR1, CDR2, and CDR3 of the sdAb sequence set forth in SEQ ID NO: 54.

In one embodiment, the sdAb comprises a CDR1, CDR2, and CDR3 of the sdAb sequence set forth in SEQ ID NO: 55.

In one embodiment, the sdAb comprises a CDR1, CDR2, and CDR3 of the sdAb sequence set forth in SEQ ID NO: 56.

In one embodiment, the sdAb comprises a CDR1, CDR2, and CDR3 of the sdAb sequence set forth in SEQ ID NO: 57.

In one embodiment, the sdAb comprises a CDR1, CDR2, and CDR3 of the sdAb sequence set forth in SEQ ID NO: 58.

In one embodiment, the sdAb comprises a CDR1, CDR2, and CDR3 of the sdAb sequence set forth in SEQ ID NO: 79.

In one embodiment, the sdAb comprises a CDR1, CDR2, and CDR3 of the sdAb sequence set forth in SEQ ID NO: 80.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In one embodiment, the sdAb comprises SEQ ID NO: 49.

In one embodiment, the sdAb comprises SEQ ID NO: 50.

In one embodiment, the sdAb comprises SEQ ID NO: 51.

In one embodiment, the sdAb comprises SEQ ID NO: 52.

In one embodiment, the sdAb comprises SEQ ID NO: 53.

In one embodiment, the sdAb comprises SEQ ID NO: 54.

In one embodiment, the sdAb comprises SEQ ID NO: 55.

In one embodiment, the sdAb comprises SEQ ID NO: 56.

In one embodiment, the sdAb comprises SEQ ID NO: 57.

In one embodiment, the sdAb comprises SEQ ID NO: 58.

In one embodiment, the sdAb comprises SEQ ID NO: 79.

In one embodiment, the sdAb comprises SEQ ID NO: 80.

In some embodiments, the BiKE or TriKE is a sequence variant of one of the above BiKEs and TriKEs having 80%, 90%, 95%, 98%, or 99% identity thereto. In some embodiments, the variant retains substantially the same binding specificity as the parent molecule from which it is derived. In some embodiments the variant retains substantially the same binding affinity as the parent molecule from which it is derived.

### Nucleic Acid Molecules

In aspect, there is provided a recombinant nucleic acid molecule encoding the multivalent antibody as defined herein. In one embodiment, the nucleic acid molecule may comprise DNA. In one embodiment, the nucleic acid molecule may comprise RNA. In one embodiment, the nucleic acid molecule may comprise mRNA. In one embodiment, the nucleic acid molecule may comprise any nucleic acids that encode a protein. In one embodiment, nucleic acid is a vector.

### Compositions

In one aspect, there is provided a composition comprising a multivalent antibody as defined herein; together with an acceptable excipient, diluent or carrier. In one embodiment, the composition comprises a bispecific T-cell engager as herein defined. In one embodiment, the composition comprises a BiKE as herein defined. In one embodiment, the composition comprises a TriKE as herein defined. In one embodiment the composition is a pharmaceutical composition, and the excipient, diluent or carrier is a pharmaceutically acceptable excipient, diluent or carrier.

### Uses & Methods

In one aspect, there is provided a use of the multivalent antibody as defined herein for treatment of a cancer or an auto-immune disease. In one embodiment, the cancer or auto-immune disease to be treated is characterized by aberrant or increased expression of BCMA relative to healthy cells. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect, there is provided a use of the multivalent antibody as defined herein for preparation of a medicament for treatment of a cancer or an auto-immune disease. In one embodiment, the cancer or auto-immune disease to be treated is characterized by aberrant or increased expression of BCMA relative to healthy cells. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect, there is provided the multivalent antibody as defined herein for use in treatment of a cancer or an auto-immune disease. In one embodiment, the cancer or auto-immune disease to be treated is characterized by aberrant or increased expression of BCMA relative to healthy cells. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect, there is provided a method of treating a cancer or an auto-immune disease in subject comprising administering to the subject the multivalent antibody as defined herein. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

### Chimeric Antibody Receptors & Related Embodiments

In one aspect, there is provided a chimeric antibody receptor (CAR), which binds to human BCMA, comprising the VHH sdAb as defined herein.

“Chimeric antigen receptors” are receptor proteins engineered to give T cells the new ability to target a specific protein. The receptors are chimeric because they combine both antigen-binding and T-cell activating functions into a single receptor (see Stoiber et al. Limitations in the Design of Chimeric Antigen Receptors for Cancer Therapy. Cells. 2012; 8(5): 472 and van der Stegen et al. The pharmacology of second-generation chimeric antigen receptors. Nat Rev Drug Discov. 2019; 14(7): 499-509).

In one embodiment, the CAR comprises, in N-terminal to C-terminal direction:
a BCMA binding domain comprising the sdAb as defined herein,
a polypeptide hinge,
a transmembrane domain, and
a cytoplasmic domain comprising a co-stimulatory domain and a signaling domain.

The term “polypeptide hinge” used herein generally means any oligo- or polypeptide that functions to link the extracellular ligand-binding domain to the transmembrane domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively the hinge region may be a synthetic sequence that corresponds to a naturally occurring hinge sequence, or may be an entirely synthetic hinge sequence.

In one embodiment, the polypeptide hinge is a CD8 hinge domain. In one embodiment, the CD8 hinge domain comprises SEQ ID NO: 71

The term “transmembrane domain” indicates a polypeptide having the ability to span a cell membrane and thereby link the extracellular portion of the CAR (which comprises the BCMA-binding portion) to the intracellular portion responsible for signaling. Commonly used transmembrane domains for CARs have been derived from CD4, CD8α, CD28 and CD3ζ.

In one embodiment, the transmembrane domain is a CD28 transmembrane domain. In one embodiment, the CD28 transmembrane domain comprises SEQ ID NO: 72. In one embodiment, the transmembrane domain is at least 80% identical to SEQ ID NO: 72. In one embodiment, the transmembrane domain is at least 90% identical to SEQ ID NO: 72. In one embodiment, the transmembrane domain is at least 95% identical to SEQ ID NO: 72. In one embodiment, the transmembrane domain is at least 98% identical to SEQ ID NO: 72.

The term “cytoplasmic domain” (also termed a “signal transduction domain”) refers to the intracellular portion of the CAR that is responsible for intracellular signaling following the binding of extracellular ligand binding domain to the target resulting in the activation of the immune cell and immune response. In other words, cytoplasmic domain is responsible for the activation of at least one of the normal effector functions of the immune cell in which the CAR is expressed. For example, the effector function of a T cell can be a cytolytic activity or helper activity including the secretion of cytokines. Thus, the term “cytoplasmic domain” refers to the portion of a protein which transduces the effector signal and directs the cell to perform a specialized function. It is common for such cytoplasmic domains to comprise a co-stimulatory domain in addition to a signaling domain.

The term “signaling domain” refers to the portion of a protein which transduces the effector signal and directs the cell to perform a specialized function. Examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transducing domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Non-limiting examples of signaling domains used in the invention can include those derived from TCRzeta, common FcR gamma (FCERIG), Fcgamma RIIa, FcRbeta (Fc Epsilon Rib), FcRepsilon, CD3 zeta, CD3gamma, CD3delta, CD3epsilon, CD5, CD22, CD79a, CD79b, CD66d, DAP10, or DAP12. In a preferred embodiment, the signaling transducing domain of the CAR can comprise the CD3zeta signaling domain.

In one embodiment, the signaling domain is a CD3-zeta signaling domain. In one embodiment, the CD3-zeta signaling domain comprises SEQ ID NO: 74. In one embodiment, the signaling domain is at least 80% identical to SEQ ID NO: 74. In one embodiment, the signaling domain is at least 90% identical to SEQ ID NO: 74. In one embodiment, the signaling domain is at least 95% identical to SEQ ID NO: 74. In one embodiment, the signaling domain is at least 98% identical to SEQ ID NO: 74.

The term “co-stimulatory domain” refers to the cognate binding partner on a T-cell that specifically binds with a co-stimulatory ligand, thereby mediating a co-stimulatory response by the cell, such as, but not limited to proliferation. Co-stimulatory molecules include, but are not limited to, an MHC class I molecule, BTLA and Toll ligand receptor. Examples of costimulatory molecules include CD27, CD28, 4-1EE (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CDIId, ITGAE, CD103, ITGAL, CDIIa, LFA-1, ITGAM, CDIIb, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D or a combination thereof.

In one embodiment, the co-stimulatory domain is a 4-1 BB co-stimulatory domain. In one embodiment, the 4-1BB signal transduction domain comprises SEQ ID NO: 73. In one embodiment, the co-stimulatory domain is at least 80% identical to SEQ ID NO: 73. In one embodiment, the co-stimulatory domain is at least 90% identical to SEQ ID NO: 73. In one embodiment, the co-stimulatory domain is at least 95% identical to SEQ ID NO: 73. In one embodiment, the co-stimulatory domain is at least 98% identical to SEQ ID NO: 73.

In one embodiment, CAR further comprises a flexible amino acid linker between the sdAb and the polypeptide hinge. In one embodiment, the amino acid linker comprises SEQ ID NO: 70. In one embodiment, the amino acid linker is at least 80% identical to SEQ ID NO: 70. In one embodiment, the amino acid linker is at least 90% identical to SEQ ID NO: 70. In one embodiment, the amino acid linker is at least 95% identical to SEQ ID NO: 70. In one embodiment, the amino acid linker is at least 98% identical to SEQ ID NO: 70.

In one embodiment, the CAR further comprises a signal peptide.

In one embodiment, the signal peptide is a signal peptide from human CD28. In one embodiment, the signal peptide from human CD28 comprises SEQ ID NO: 69. In one embodiment, the signal peptide is at least 80% identical to SEQ ID NO: 69. In one embodiment, the signal peptide is at least 90% identical to SEQ ID NO: 69. In one embodiment, the signal peptide is at least 95% identical to SEQ ID NO: 69. In one embodiment, the signal peptide is at least 98% identical to SEQ ID NO: 69.

In one embodiment, the CAR is encoded by SEQ ID NO: 68.

In one embodiment, the sdAb comprises a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7).

In one embodiment, the sdAb comprises a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2).

In one embodiment, the sdAb comprises a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3).

In one embodiment, the sdAb comprises a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5).

In one embodiment, the sdAb comprises a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4).

In one embodiment, the sdAb comprises a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1).

In one embodiment, the sdAb comprises a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2).

In one embodiment, the sdAb comprises a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6).

In one embodiment, the sdAb comprises a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)).

In one embodiment, the sdAb comprises a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2).

In one embodiment, the ant sdAb comprises a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)).

In one embodiment, the sdAb comprises a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10).

In one embodiment, the sdAb comprises a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2).

In one embodiment, the sdAb comprises a CDR1, CDR2, and CDR3 of the sdAb sequence set forth in SEQ ID NO: 40.

In one embodiment, the sdAb comprises a CDR1, CDR2, and CDR3 of the sdAb sequence set forth in SEQ ID NO: 41.

In one embodiment, the sdAb comprises a CDR1, CDR2, and CDR3 of the sdAb sequence set forth in SEQ ID NO: 42.

In one embodiment, the sdAb comprises a CDR1, CDR2, and CDR3 of the sdAb sequence set forth in SEQ ID NO: 43.

In one embodiment, the sdAb comprises a CDR1, CDR2, and CDR3 of the sdAb sequence set forth in SEQ ID NO: 44.

In one embodiment, the sdAb comprises a CDR1, CDR2, and CDR3 of the sdAb sequence set forth in SEQ ID NO: 45.

In one embodiment, the sdAb comprises a CDR1, CDR2, and CDR3 of the sdAb sequence set forth in SEQ ID NO: 46.

In one embodiment, the sdAb comprises a CDR1, CDR2, and CDR3 of the sdAb sequence set forth in SEQ ID NO: 47.

In one embodiment, the sdAb comprises a CDR1, CDR2, and CDR3 of the sdAb sequence set forth in SEQ ID NO: 48.

In one embodiment, the sdAb comprises a CDR1, CDR2, and CDR3 of the sdAb sequence set forth in SEQ ID NO: 49.

In one embodiment, the sdAb comprises a CDR1, CDR2, and CDR3 of the sdAb sequence set forth in SEQ ID NO: 50.

In one embodiment, the sdAb comprises a CDR1, CDR2, and CDR3 of the sdAb sequence set forth in SEQ ID NO: 51.

In one embodiment, the sdAb comprises a CDR1, CDR2, and CDR3 of the sdAb sequence set forth in SEQ ID NO: 52.

In one embodiment, the sdAb comprises a CDR1, CDR2, and CDR3 of the sdAb sequence set forth in SEQ ID NO: 53.

In one embodiment, the sdAb comprises a CDR1, CDR2, and CDR3 of the sdAb sequence set forth in SEQ ID NO: 54.

In one embodiment, the sdAb comprises a CDR1, CDR2, and CDR3 of the sdAb sequence set forth in SEQ ID NO: 55.

In one embodiment, the sdAb comprises a CDR1, CDR2, and CDR3 of the sdAb sequence set forth in SEQ ID NO: 56.

In one embodiment, the sdAb comprises a CDR1, CDR2, and CDR3 of the sdAb sequence set forth in SEQ ID NO: 57.

In one embodiment, the sdAb comprises a CDR1, CDR2, and CDR3 of the sdAb sequence set forth in SEQ ID NO: 58.

In one embodiment, the sdAb comprises SEQ ID NO: 40.

In one embodiment, the sdAb comprises SEQ ID NO: 41.

In one embodiment, the sdAb comprises SEQ ID NO: 42.

In one embodiment, the sdAb comprises SEQ ID NO: 43.

In one embodiment, the sdAb comprises SEQ ID NO: 44.

In one embodiment, the sdAb comprises SEQ ID NO: 45.

In one embodiment, the sdAb comprises SEQ ID NO: 46.

In one embodiment, the sdAb comprises SEQ ID NO: 47.

In one embodiment, the sdAb comprises SEQ ID NO: 48.

In one embodiment, the sdAb comprises SEQ ID NO: 49.

In one embodiment, the sdAb comprises SEQ ID NO: 50.

In one embodiment, the sdAb comprises SEQ ID NO: 51.

In one embodiment, the sdAb comprises SEQ ID NO: 52.

In one embodiment, the sdAb comprises SEQ ID NO: 53.

In one embodiment, the sdAb comprises SEQ ID NO: 54.

In one embodiment, the sdAb comprises SEQ ID NO: 55.

In one embodiment, the sdAb comprises SEQ ID NO: 56.

In one embodiment, the sdAb comprises SEQ ID NO: 57.

In one embodiment, the sdAb comprises SEQ ID NO: 58.

In one embodiment, the sdAb comprises SEQ ID NO: 79.

In one embodiment, the sdAb comprises SEQ ID NO: 80.

In one embodiment, the CAR further comprises a second BCMA binding domain positioned N-terminally or C-terminally with respect to the first BCMA binding domain, and may be spaced apart from the first BCMA binding domain by an amino acid linker.

In one embodiment, the second BCMA binding domain comprises and sdAb that is the same as the sdAb of the first BCMA binding domain. These embodiments are referred to herein as “double binders”.

In another embodiment, the second BCMA binding domain comprises an sdAb that is different to the sdAb of the first BCMA binding domain. These embodiments are referred to herein as “bi-paratopic”. In this embodiment, the sdAb of the second BCMA binding domain may bind to a different epitope of BCMA to that bound by the sdAb of the first BCMA binding domain. A “different epitope” may alternatively be an epitope that overlaps that bound by the sdAb of the first BCMA binding domain. Alternatively, the sdAb may bind to the same epitope to that bound by the sdAb of the first BCMA binding domain.

In one embodiment, the CAR further comprises an additional binding domain that binds to a target molecule other than BCMA. These embodiments are referred to herein as “tandem constructs”. The additional binding domain may comprise an additional sdAb or an ScFv. The additional binding domain may be positioned N-terminally or C-terminally with respect to the BCMA binding domain. The additional binding domain may be separated from the BCMA binding domain by an amino acid linker. In one embodiment, the target molecule bound by the additional binding domain is expressed by a target cell that also expresses BCMA, thereby providing a CAR having dual affinity for the same target cell. For example, the target molecule other than BCMA may be CD19, CD20, CD22, CD44v6, GPRC5D, or integrin beta 7.

In some embodiments, the tandem constructs may comprise a third binding domain that targets yet another target molecule distinct from BCMA and distinct from that bound by additional binding domain. Such constructs are referred to herein as “multi-binders”.

In some embodiments, the CAR is a sequence variant of one of the above CARs having 80%, 90%, 95%, 98%, or 99% identity thereto. In some embodiments, the variant retains substantially the same binding specificity as the parent molecule from which it is derived. In some embodiments the variant retains substantially the same binding affinity as the parent molecule from which it is derived.

Nucleic Acids & Vectors

In one aspect, there is provided a nucleic acid molecule encoding the CAR as defined herein. In one embodiment, the nucleic acid molecule may comprise DNA. In one embodiment, the nucleic acid molecule may comprise RNA. In one embodiment, the nucleic acid molecule may comprise mRNA. In one embodiment, the nucleic acid molecule may comprise any nucleic acids that encode a protein. In one embodiment, nucleic acid is a vector.

In one aspect, there is provided a vector comprising the recombinant nucleic acid molecule as defined herein. In one embodiment, the vector is a viral vector. In one embodiment, the viral vector is a lentivirus vector.

### Viral Particles

In one aspect, there is provided a recombinant viral particle comprising the recombinant nucleic acid as defined herein. In one embodiment, the recombinant viral particle is a recombinant lentiviral particle.

### Cells

In one aspect, there is provided a cell comprising the recombinant nucleic acid molecule as defined herein.

In one aspect, there is provided an engineered cell expressing at the cell surface membrane the CAR as defined herein. In one embodiment, the engineered cell is an immune cell. In one embodiment, the immune cell is a T-lymphocyte or is derived from T-lymphocytes.

### Use & Methods

“CAR-T” cell therapy uses T cells engineered with CARs for cancer therapy. The premise of CAR-T immunotherapy is to modify T cells to recognize disease cells, typically cancer cells, in order to more effectively target and destroy them. Generally, T are genetically altered to express a CAR, and these cells are infused into a patient to attack their tumors. CAR-T cells can be either derived from T cells in a patient's own blood (autologous) or derived from the T cells of another healthy donor (allogeneic).

In one aspect, there is providing a use of the nucleic acid, vector, or viral particle as described herein for preparation of cells for CAR-T.

In one aspect, there is providing a method of preparing cells for CAR-T comprising contacting a T-cell with the viral particle as described herein. In one embodiment, the T-cell is from a donor. In one embodiment, the T-cell is from a patient.

In one aspect, there is providing a method of preparing cells for CAR-T comprising introducing into a T-cell the nucleic acid or vector as described herein. In one embodiment, the T-cell is from a donor. In one embodiment, the T-cell is from a patient.

In one aspect, there is provided a use of the CAR or of the engineered cell as described herein for treatment of a cancer or an auto-immune disease. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one embodiment, the method further comprises an initial step of obtaining cells from a patient or donor and introducing the recombinant nucleic acid molecule or vector encoding the CAR, as described herein.

In one embodiment, the method further comprises an initial step of obtaining cells from a patient or donor and contacting the cells with the viral particle, as described herein.

In one aspect, there is provided a use of the CAR or of the engineered cell as described herein for preparation of a medicament treatment of a cancer or an auto-immune disease. In one embodiment, the cancer or auto-immune disease to be treated is characterized by aberrant or increased expression of BCMA relative to healthy cells. In one embodiment, the cancer is a hematological malignancy. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect, there is provided the CAR or the engineered cell as described herein for use in treatment of a cancer or an auto-immune disease. In one embodiment, the cancer or auto-immune disease to be treated is characterized by aberrant or increased expression of BCMA relative to healthy cells. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

In one aspect there is provided a method of treating a cancer or an auto-immune disease in a subject, comprising administering to the subject the engineered cell as defined herein. In one embodiment, the cancer or auto-immune disease to be treated is characterized by aberrant or increased expression of BCMA relative to healthy cells. In one embodiment, the hematological malignancy is multiple myeloma (MM), lymphoma, chronic lymphocytic leukemia (CLL), B-cell acute lymphoblastic leukemia (B-ALL), or acute myelogenous leukemia (AML). In one embodiment, the hematological malignancy is multiple myeloma or lymphoma. In one embodiment, the lymphoma is diffuse large B cell lymphoma (DLBCL), non-Hodgkin lymphoma (NHL), Hodgkin Lymphoma (HL), plasmablastic lymphoma, Burkitt's lymphoma, marginal zone lymphoma (MZL), or mantle cell lymphoma (MCL).

### EXAMPLES

The following Examples outline embodiments of the invention and/or studies conducted pertaining to the invention. While the Examples are illustrative, the invention is in no way limited the following exemplified embodiments.

B-cell maturation antigen (BCMA) is expressed at significantly higher levels in all patient MM cells but not on other normal tissues except on surface of plasmablasts and differentiated plasma B cells. Although BCMA targeted immunotherapy may eliminate healthy plasma B cells; the resulting side effects of this is easily managed with passive immunoglobulin treatments.

BCMA has emerged as a molecular target of intense interest, with researchers developing new BCMA-targeted treatments using naked antibodies, chimeric antigen receptor-T cells (CAR-T), bi-specific T cell engagers (BITE), and others. Blinatumomab was the first BiTE which shown efficacy in in patients, in particular, with relapsed/refractory B-ALL. BCMA CAR-T-based scFv and single domain antibodies (sdAbs) have also been developed and shown variable degree of efficacy in multiple myeloma patients. Notable examples are a CAR-T molecule developed in China harbouring two llama-sdAbs which target two different BCMA epitopes with impressive efficacy in two clinical trials performed at different sites in China, followed by a recent follow-up trial sponsored by Janssen wherein deep MRD-negative responses were observed leading to breakthrough drug review status and approval from the FDA. The use of sdAbs in the CAR format have significant advantages over traditional scFv-based CARs including: (a) smaller size which makes them less immunogenic, (b) single-molecular structure eases cloning and incorporation in larger and more complex molecules, and (c) targeting of cancer-associated or other novel epitopes otherwise not targetable with scFvs.

B cell directed therapies have also proven to be effective in treating autoimmune diseases including classic B cell/autoantibody-driven disorders, such as systemic lupus erythematosus (SLE), autoimmune blistering skin diseases, myasthenia gravis and T cell driven autoimmune diseases such as rheumatoid arthritis (RA) or multiple sclerosis (MS). BCMA is a key regulators of B cell proliferation and survival, as well as maturation and differentiation into plasma cells. Thus BCMA targeted therapies may also be clinically effective in treating B cell mediated autoimmune diseases.

The applicability of camelid single domain antibodies as soluble, stable and modular domains for a number of therapeutic applications has well-been established with the first FDA-approved bivalent nanobody in 2018. Therefore, nanobodies present an excellent building block in CAR-T molecules, allowing a simple antibody domain fusion and building a pool of more stable and functional CAR-T constructs, therefore, increasing the chance of screening much more effective CAR-T cells for the treatment of non-solid tumor cells.

In addition these nanobodies could also be utilized to develop additional safe and efficacious immunotherapy regimens including but not limited to naked or drug conjugated antibody therapies and specific immune cell engager therapeutics.

The approaches described herein use single domain antibodies (sdAb) derived from an immunized llama with a unique BCMA-ECD protein fusion strategy developed at the NRC-HHT. These sdAb sequences specifically bind to BCMA antigen with high affinities which is preferentially expressed by mature B lymphocytes and its activation and overexpression are associated with multiple myeloma in preclinical models and in humans. Using the sdAb sequences, a novel chimeric receptor sequence has been generated that combines BCMA specific sdAb with T cell signaling molecules (in the form of 41BB, CD28 or other co-stimulation domain and CD3zeta signaling domains). In addition to chimeric antigen receptor applications, these BCMA targeting antibodies may be useful for developing other forms of immunotherapies including but not limited to bi-specific/tri-specific T or NK cell engager applications, antibody-drug conjugates, or as naked antibodies.

### Example 1: sdAb Antibody Production

### Introduction

Single domain antibodies (sdAbs) (also known as VHHs or nanobodies) derived from the variable domains of the camelid heavy chain, are characteristically stable and fully capable of antigen binding in the absence of the former VL domain. In addition to their small size, sdAbs possess high affinity, high solubility, and low immunogenicity in humans due to their high homology to human VH3 family, high expression levels in microorganisms such as bacteria and yeast, and remarkable stability at high temperature, extreme pH and high salt concentrations. Due to their superb antibody engineering potential, sdAbs are considered as ideal building blocks for bi- and multi-specific therapeutic reagents. Notable examples include the first FDA-approved bivalent anti-vWF nanobodies (Caplacizumab, 2019) and ten other therapeutic nanobodies, in bi-/multi-valent or bi-/multi-specific formats, which have been advanced into pre-clinical and clinical development by Ablynx/Sanofi and other biopharmaceutical companies thus far.

sdAbs are also ideal building blocks for the generation of Chimeric Antigen Receptor (CAR), whereby cancer-specific antigen binding domains (scFv, Fab) of conventional IgGs are genetically fused with immune T-cell activating domains to generated “armored” Immune T lymphocytes (CAR-T) that seek and kill specific cells that harbor the targeting antigen(s). Applying sdAbs in CAR-T constructs reduces domain complexity of scFv/Fab fragments and significantly increases the productivity and effectiveness of the final CAR-T constructs. It also allows additional specificity (against a second cancer biomarker or a different epitope on the same biomarker) to be added to the CAR-T construct (i.e., to generate bi-specific CAR-T cell), therefore, increasing the chance of generating much more effective CAR-T cells for the treatment of haematological tumors. Similarly, these sdAbs are ideal candidates for the development of other forms of immunotherapies such as bi-, tri- and multi-specific immune cell engagers.

In this study, functional camelid sdAbs are generated against the ecto-domian of BCMA that is preferentially expressed by mature B lymphocytes and its activation and overexpression are associated with multiple myeloma in preclinical models and in humans. The sdAbs will then be used to develop immunotherapeutics including but not limited to CAR-T therapies, bi-, tri- and multi-specific immune engager therapies, and naked or drug/tracer linked therapeutic antibodies with appropriate human IgG fusions. The sdAb may also be used to target other therapeutic modalities to MM cells. These therapies are intended for use as treatment modalities for cancer, auto-immune and inflammatory diseases. Examples are presented of the use of these sdAb sequences for developing CAR-T and bi-specific immune engagers with effective anti-tumor activity.

### Materials and Methods

### Cloning and Expression of BCMA-ECD

The gene encoding the extracellular domain of human predominant BCMA isoform 1 was fused to either mouse IgG2a-Fc (mIgG2a-Fc) or to a VHH carrier protein (FC5) and cloned into pTT5™ NRC proprietary mammalian expression vector. Upon transfection of HEK-293 cells, the cells were grown in a 250 mL flask and the expressed proteins were purified by Protein A column (MabSelect™ SuRe™) and Immunoaffinity chromatography (IMAC) and analyzed on SDS-PAGE.

### Llama Immunizations

A llama (LPAR1) was immunized with the BCMA-ECD-Fc (Protein Production Team, HHT-Montreal) and subsequently boosted with the recombinant human and mouse BCMA-ECD-FC5 (hBCMA-ECD-FC5 and mBCMA-ECD-FC5) antigens (NRT-HHT-sdAb Team). For each injection, 100 μg of recombinant mIgG2a-Fc/hBCMA-ECD-FC5/mBCMA-ECD-FC5, in a total volume of 0.5 mL was mixed with an equal volume of complete (first injection) and incomplete Freund's adjuvant (subsequent injections) and was injected, subcutaneously. Five injections were performed at approximately two week intervals and blood was collected after the third injection and 7 days after the last injection.

### RNA Isolation and PCR Amplification

Total RNA was isolated from approximately 2×10⁷ lymphocytes collected from day 49 of the immunization protocol with a QIAamp RNA blood mini kit (QIAGEN Sciences, Mississauga, ON) and according to the kit instructions. About 5 μg of total RNA was used as template for first strand cDNA synthesis with an oligo dT primer using a first-strand cDNA synthesis kit (Amersham Biosciences, USA). Based on the Camelidae and llama immunoglobulin databases, three variable domain sense primers (MJ1-3) and two CH2 domain antisense primers (CH2 and CH2b3) were designed (Baral T N et al 2013). The first PCR was performed with the cDNA as template and the variable regions of both conventional (IgG1) and heavy chain antibodies (IgG2 and IgG3) were amplified with combinations of MJ1-3/CH2 and MJ1-3/CH2b primers in two separate reactions. The PCR reaction mixtures contained the following components: 2 μL cDNA, 5 pmol of MJ1-3 primer mixture, 5 pmol of either CH2 or CH2b primer, 5 μL of 10× reaction buffer, 3 μL of 2.5 mM dNTP, 2.5 units of Taq DNA polymerase (Roche Applied Science, Indianapolis, IN) and water to a final volume of 50 μL. The PCR protocol consisted of an initial step at 94° C. for 3 minutes followed by 30 cycles of 94° C. for 30 seconds, 55° C. for 30 seconds, 72° C. for 1 minute and a final extension step at 72° C. for 7 minutes. The amplified PCR products were run onto a 2% agarose gel and consisted of two major bands of about 850 bp corresponding to conventional IgG1 and about 600 bp (550-650 bp) corresponding to heavy chain antibodies. The smaller bands were cut out of the gel, purified with a QIAquick gel extraction kit (QIAGEN Inc) and re-amplified in a second PCR reaction containing 1 μL of the purified DNA template, 5 pmol each of MJ7, a VH sense primer with a Sfil restriction site, underlined,

(5′-CAT GTG TAG ACT CGC GGC
CCA GCC GGC CAT GGC C-3′)

and MJ8, an antisense primer with a Sfil restriction enzyme site, underlined,

(5′-CAT GTG TAG ATT CCT GGC
CGG CCT GGC CTG AGG AGA CGG
TGA CCT GG),

5 μL of 10× reaction buffer, 3 μL of 2.5 mM dNTP, 2.5 unit of Taq DNA polymerase (Roche Applied Science, Indianapolis, IN) and water to a final volume of 50 μL. The PCR protocol consisted of an initial step at 94° C. for 3 minutes followed by 30 cycles of 94° C. for 30 seconds, 57° C. for 30 seconds, 72° C. for 1 minute and a final extension step at 72° C. for 7 minutes. The amplified PCR products (about 400-450 bp) that correspond to VHH fragments of heavy chain antibodies were purified with a QIAquick PCR purification kit (QIAGEN Inc.), digested with Sfil (New England BioLabs) and re-purified with the same kit.

### Library Construction

Thirty μg of pMED1 (Arbabi-Ghahroudi et al. 2009) DNA was digested with Sfil overnight at 50° C. To minimize the chance of self-ligation, the digestion was continued for additional 2 hours at 37° C. by adding 20 units of both XhoI and Pst/restriction enzymes. For library construction, 10 μg of phagemid DNA was ligated with 1.75 ug of VHH fragments and incubated for 2 hours at room temperature using the LigaFast DNA ligation system (Promega, Corp., Madison, WI) and according to the recommended protocol. The ligated product was electroporated into competent E. coli TG1cells (Stratagene, Cedar Creek, TX). Transformed bacterial cells were diluted in SOC medium and incubated for 1 hour at 37° C. with slow shaking. The size of library was calculated by plating aliquots on LB-Amp. The VHH fragments from 96 colonies were PCR-amplified and sequenced for diversity analysis. The library was aliquoted and stored at −80° C.

### Library Panning and Screening

The constructed LPAR1 Library with an approximate size of 2×10⁷ was phage-recued and the phage titer of 1.0×10¹⁰ cfu/uL was used to pan against the in vivo biotinylated hBCMA-FC5 or mBCMA antigen. Four rounds of panning was performed with alternating human and mouse BCMA as well as blocking buffers [e.g. Starter Block (Thermo Fisher Cat #37559) for rounds1, 3 and biotin-free casein for rounds 2, 4. Panning was also alternated between both Pierce™ streptavidin coated wells (Round 1, 3) (Thermoscientific cat #15501; lot #TF252884) and Pierce™ neutravidin coated wells (Round 2, 4) (Thermoscientific cat #15508; lot #SK253835). One neutravidin well was rinsed with 100 μL PBS and coated with 1 μg of biotinylated human or mouse BCMA-FC5 (well #3) and second well (negative control) (well #1) was filled with the PBS only and the plate was incubated at 37° C. for 1 hr. Additionally, one well (well #2) in an Immulon 4HBX plate was also coated with 5 μg FC5VHH (the llama VHH fusion protein) and incubated for 1 hr at 37° C. All three wells were blocked with Starting block for 1 hr at 37° C. and then rinsed with 300 μL PBC.

Phage Library input phage (˜1×10¹²) was added to the well #1 and incubate 1 hr at room temperature. The input phages (supernatant of well #1) were transferred to the well #2 (Immulon 4HBX plate) and incubated for an additional 1 hr at room temperature. The phage supernatant were then transferred to the antigen well (well #3) and incubated for 1 hr at room temperature. This was followed with wash steps 13×300 μL PBS-T (PBS+0.05% Tween 20) (quick); 2×300 μL PBS-T+(PBS+0.05% Tween 20) (incubate 5 minutes each wash); 3×300 μL PBS (quick); 2×300 μL PBS (incubate 5 minutes each wash)} and elution with 100 μL of 100 mM TEA. Phage were then removed from wells and neutralized with 50 μL of 1M Tris-HCl pH 7.4 in a new tube. 3 mL of exponentially growing TG1 E. coli culture previously grown at 37° C., 250 rpm, until OD₆₀₀=0.5 in 2YT+2% glucose in a 15 mL Falcon tube, was infected with the eluted phage. A 100 μL aliquot of uninfected TG1 E. coli cells was set aside as a control. Eluted phage were Incubated at 37° C. for 30 minutes with no shaking and then an aliquot was used for titer (dilutions of 10² to 10⁸) and plating on 2YT plates overnight at 32° C. The remaining 3 mL of infected TG1 culture, proceeded with overnight phage amplification using M13KO7 helper phage (˜1×10¹⁰ cfu).

The next day the eluted titers were calculated to determine the amount of input phage for the subsequent round. The cell culture containing the amplified phage was centrifuged at 5000 rpm, 30 minutes and the supernatant was filtered through 0.22 uM filter unit (Millipore) and precipitated in 20% PEG/2.5M sodium chloride (NaCl) followed by centrifugation and re-solubilization in PBS (pH 7.5). Amplified phage titer was determined (dilutions of 10⁴ to 10¹²) in TG1 E. coli cells as grown previously. The panning was repeated for three more rounds as described above but the washing conditions was more stringent as described elsewhere (Baral T N et al 2013). In subsequent rounds, ˜1×10¹² of input phage from each round of amplified phage was used.

After 4 rounds of panning, the sequences of positive colonies from phage ELISA were aligned and analyzed to identify the unique VHH sequences.

### Results

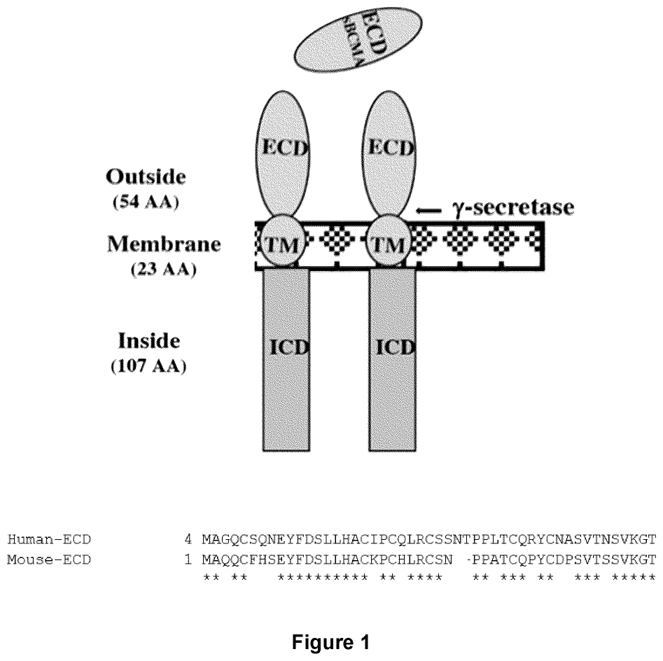

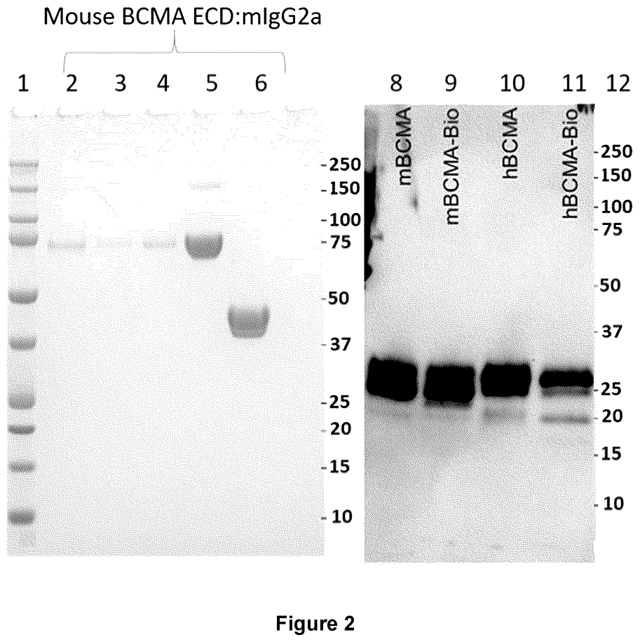

The extracellular domain (ECD) of human BCMA which include a single domain of 54 amino acids (Genbank Accession BAB60895 or UniProtKB Accession Q022223) (FIG. 1) was fused to the Fc region of mouse IgG2a (mIgG2a-BCMA). The ECD domain of human and mouse BCMA (49 amino acids; see, amino acids 1 to 49 of Genbank Accession AAC23799 or UniProtKB Accession 088472) were also fused to a camelid VHH (FC5). All constructs were cloned into pTT5 mammalian expression vector with a 19 amino acid leader signal. The mIgG2a-BCMA and BCMA-FC5VHH proteins were expressed in CHO and HEK-293 cells, respectively (NRC-HHT Montreal & Ottawa). The expressed mIgG2a-BCMA protein in CHO cells (290 aa; 31.9 kDa) was purified by protein A column (MabSelect™ SuRe™) under DTT-reducing and non-reducing conditions (FIG. 2, left panel). The human and mouse BCMA-FC5VHH fusion proteins (222 aa; 24.4 kDa and 217 aa; 23.9 kDa, respectively) were purified by Immunoaffinity chromatography (IMAC) and analyzed on SDS-PAGE under DTT-reducing conditions (FIG. 2, right panel).

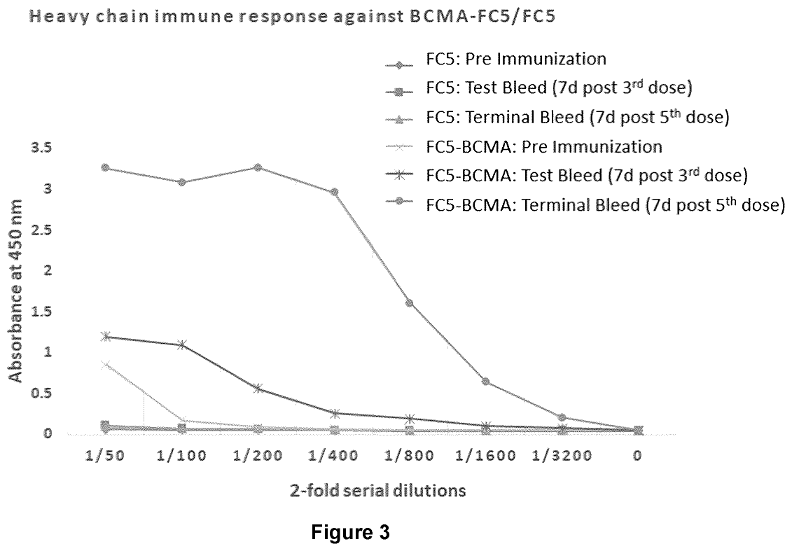

The recombinant mIgG2a-BCMA was used to immunize a llama (LPAR1) along with some additional proteins (CD69 and CRLF2)) and the llama immune response was monitored and analyzed by ELISA using alternative hBCMA-FC5VHH as the coating antigen. As shown in FIG. 3, BCMA-ECD injection elicited a strong heavy chain immune response in llama when it compared with the other two antigens used in immunization. The immune response to the FC5VHH (the fusion partner) is also minimal which indicates a heavy chain response is largely directed to the BCMA-ECD domain. The heavy chain immune response in llama's serum is measured by the use two monoclonal antibodies (mAbs; NRC in-house; unpublished results) which specifically bind to the heavy chain IgG2 and IgG3 llama sub-classes.

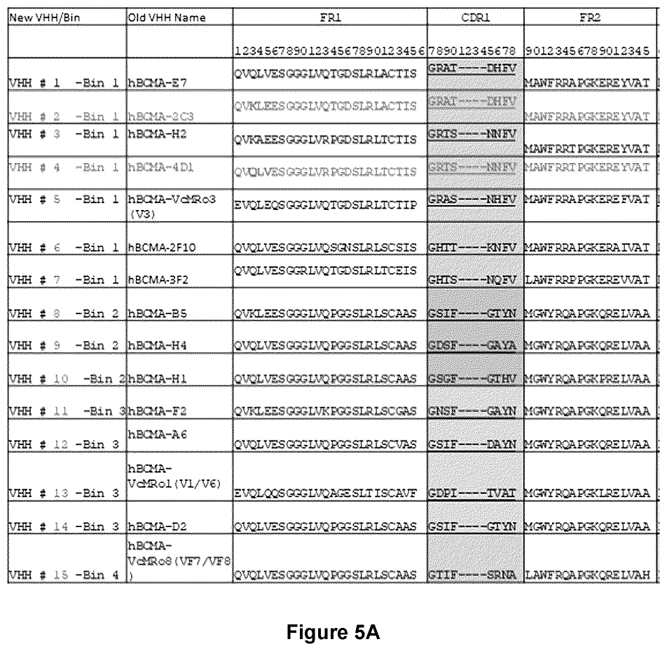

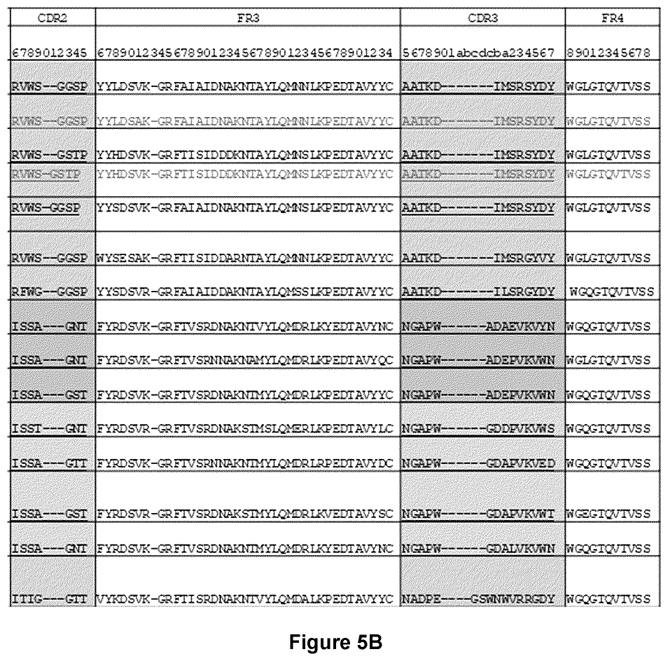

The heavy chain repertoire of llama immunoglobulins was amplified by gene-specific primers and cloned into a phagemid vector (pMED1). A medium size library (2×10⁷) was constructed and its complexity was analyzed by sending 96 colonies for sequencing. The sequencing data showed that the library has high complexity as all the VHH sequences were full-length with no repeating sequences. The library was phage-rescued using M13 helper phage as described elsewhere (Baral T N, MacKenzie R, Arbabi Ghahroudi M. Single-domain antibodies and their utility. Curr Protoc Immunol. 2013 Nov. 18; 103:2.17.1-2.17.57) and the phage antibodies were used in panning experiments where biotinylated BCMA-FC5VHH were captured on a solid surface. After four rounds of panning, 96 colonies from each panning strategy were grown and superinfected by M13 helper phage as described elsewhere (Baral T N et al 2013) and the phages were used in ELISA. Positive colonies were sent for sequencing and the sequencing data were analyzed. Alignment of the sequences was done using OPIG software and IMGT numbering (see FIGS. 5A and 5B). Based on the table provided, 13 unique VHH sequences were selected for gene synthesis and cloning into an NRC in-house expression vector (pMRO).

FIG. 1 depicts the structure of human BCMA molecule (known also as tumor necrosis factor receptor superfamily member 17; TNFRSF17). BCMA isoform 1 is the predominant isoform with 184 aa and it is a type Ill transmembrane protein with no signal peptide at the N-terminus and could also be available in soluble form (sBCMA) after it is cleaved by γ-secretase. The cytoplamic tail (107 aa) is connected by the transmembrane region (23 aa) to the extracellular domains (54 aa) (Bera 2020). The mouse BCMA has a similar structure but a with slightly shorter ECD domain (49 aa) and is connected to transmembrane region (21aa) and to the cytoplasmic tail (115 aa). There is a 71.4% identity at the ECD domain and 64.5% overall sequence identity between mouse and human BCMA.

FIG. 2 depicts a SDS-PAGE of Protein A (MabSelect™ SuRe™) and IMAC-purified BCMA extracellular domain fusion proteins (mIgG2a-BCMA, hBCMA-ECD-FC5VHH and mBCMA-ECD-FC5VHH) under reducing and non-reducing conditions. The purified proteins have the expected molecular weight of approximately 31.9 kDa (mIgG2a-BCMA) and 24.4 (hBCMA-ECD-FC5VHH) and 23.9 (mBCMA-ECD-FC5VHH).

FIG. 3 depicts the llama heavy chain immune response form the final bleed (3^{rd} August) against BCMA-ECD and two other antigens (CD69 and CRLF2)). As control, llama pre-immune serum and FC5VHH were used and no significant responses either in the pre-immune serum against BCMA-ECD or in the final bleed to the FC5VHH were observed. The binding of heavy chain antibodies was detected by anti-llama mAbs (in-house NRC) followed by donkey-anti-mouse-HRP. As shown, there is a strong and specific anti-BCMA-ECD heavy chain immune response.

### Discussion

The extracellular domain of the predominant human BCMA isoform 1 was successfully expressed in mammalian CHO and HEK-293 cell systems and the recombinant BCMA-ECD performed well in all downstream analytical assays (data not shown). This novel strategy of immunization and panning will be protected under a separate IP filing. The fusion of BCMA to a llama VHH (FC5) help to direct the llama immune response to the BCMA-ECD domain as it is expected that little or no immune response would be generated toward the llama FC5VHH as a fusion partner. After immunizing a llama with the recombinant mIgG2a-BCMA and boosted with hBCMA-ECD-FC5VHH fusion proteins, a strong heavy chain immune response was generated as determined by ELISA using heavy chain-specific mAbs. By constructing a library on the heavy chain repertoire, it was possible to isolate VHH domain antibodies specific to the immunogen (BCMA-ECD).

### Example 2: sdAb Characterization

### Introduction

Library construction on the heavy chain repertoire of immunized llama was performed following obtaining a positive immune response against the human BCMA-ECD. More than two hundred individual colonies were screened by phage-ELISA after performing a biotinylated panning strategy where in vivo biotinylated BCMA-ECD proteins were captured on a streptavidine/neutravidin surface and exposed to the rescued library phages. The individual VHH clones were sequenced and grouped based on their CDR1-3 sequences, resulting in 13 unique VHH sequences. The gene-encoding these VHHs were cloned into an NRC bacterial expression vector and purified proteins were characterized.

### Materials and Methods

### Expression of Soluble V_{H}H

The DNA sequences of the most repeated clones with phage ELISA OD₄₅₀>0.8 were sent for Gene synthesis to TWIST Bioscience and subsequently cloned into pMRO (a pET28a derivative, Novagen) expression vector. E. coli BL21(DE3) cells were transformed with the VHH constructs and the respective clones were grown in 0.25-liter cultures of 2×YT medium+ampicillin (100 mg·mL-1) with 0.1% glucose to an OD600 of 0.8. Cultures were induced with 1 mM IPTG and grown overnight on a rotary shaker at 37° C. After confirming of expression by SDS-PAGE and Western blotting, recombinant VHH proteins were extracted from the bacterial cells by standard lysis methods and purified by immobilized metal affinity chromatography (IMAC) and quantified as described elsewhere (Baral & Arbabi-Ghahroudi 2012). The VHH proteins were run on a Supdex 75 Size exclusion chromatography and the monomeric fractions were collected.

### SPR Analysis

For surface Plasmon resonance, 15 selected VHHs were passed though size exclusion columns, Superdex 75 (GE Healthcare), respectively, in 10 mM HEPES, pH 7.4, containing 150 mM NaCl, 3 mM EDTA, monomeric sdAb fractions were collected and protein concentrations were determined by measuring absorbance at 280 nm (A280). Analysis were performed with Biacore T200 instrument (GE Healthcare). All measurements were carried out at 25° C. in 10 mM HEPES, pH 7.4, containing 150 mM NaCl, 3 mM EDTA and 0.005% surfactant P20 (GE Healthcare). Approximately 500 RUs of the recombinant monomeric BCMA-ECD (obtained after SEC purification of the BCMA-ECD-FC5VHH) were captured on SA sensor chip (GE Healthcare) at a flow rate of 5 uL/min. Various concentration of the monomeric VHHs (20-500 nM) were injected over BCMA-ECD surface, respectively using an SA surface as a reference at a flow rate of 40 μL/min. Surfaces were generated by washing with running buffer. Data were analysed with BIAevaluation 4.1 software.

### Epitope Binning by SPR

In addition to obtaining binding kinetic data, Biacore co-injection experiments were performed on 4 selected VHHs (the 15 VHHs sequences were grouped into four bins based on their sequence identities and representative of each bin was used in SPR epitope binning) to determine whether these anti-BCMA VHHs could bind unique or overlapping epitopes on BCMA-ECD protein surface. Briefly, 80 μL of the first VHH diluted in HBS-EP buffer to a concentration of 5 times its KD value and was injected over 500 RUs of immobilized BCMA-ECD at 40 μL/min. Following injection of the first VHH, buffer or a second VHH (80 μL total volume, at 5×KD) was injected at 40 μL/min over the BCMA-ECD surface already saturated with the first VHH. Data were collected on all possible paired combinations of 4 VHHs, in both orientations (i.e. each VHH acted as the first and second VHH) and evaluated as described above. The epitope mapping of BCMA-2C3 was identical to BCMA E7 as the CDRs are identical in both VHHs. Likewise, the epitope mapping of H2 and 4D1 VHH is identical due to the same CDR sequences in both VHHs.

### Epitope Mapping Using Yeast Surface Display

The hBCMA ecto-domain (ECD) and its derived fragments were expressed and covalently displayed on the surface of yeast cell using the yeast surface display (Feldhaus et al., 2003). The YSD vector (pPNL6) was from The Pacific Northwest National Laboratory, USA. Twenty one hBCMA fragments covering the entire hBCMA-ECD (54aa) with overlapping ends, along with the full-length hBCMA-ECD were cloned and expressed as fusion proteins (Aga2-HA-(hBCMA)-MYC on the yeast cell surface. The displayed hBCMA fragments were used to map the regions of hBCMA to which the anti-hBCMA sdAbs of Example 1 bind. The binding of the sdAbs (biotinylated) to BCMA fragments on yeast cells was performed using a whole yeast cell ELISA probed with HRP-conjugated streptavidin. The relative amount of the displayed fusion protein was measured by probing with an anti-MYC antibody, followed by an HRP-conjugated secondary antibody, and used to normalize the binding signal for the sdAbs. The HRP activity was assayed with substrate TMB (tetramethyl benzidine) according to the manufacture's conditions and read at OD₄₅₀.

Evaluating Target Specificity of sdBCMA VHH

Purified VHH were used to assess the target specificity of the sdBCMA Ab by flow cytometry. The highly BCMA expressing human myeloma cell line RPM18226, the BCMA-low human Burkitt's lymphoma cell line Raji, and the BCMA-negative Jurkat human T cell leukemia cell line were incubated with 5 fold dilution of biotin labelled sdBCMA VHH from 7.5-0.06 μg/mL. The binding of the BCMA-targeted VHH to cell surface BCMA was detected by flow cytometry using a mixture of two broad reactivity mouse anti-VHH antibodies conjugated with AlexaFluor647.

### Results

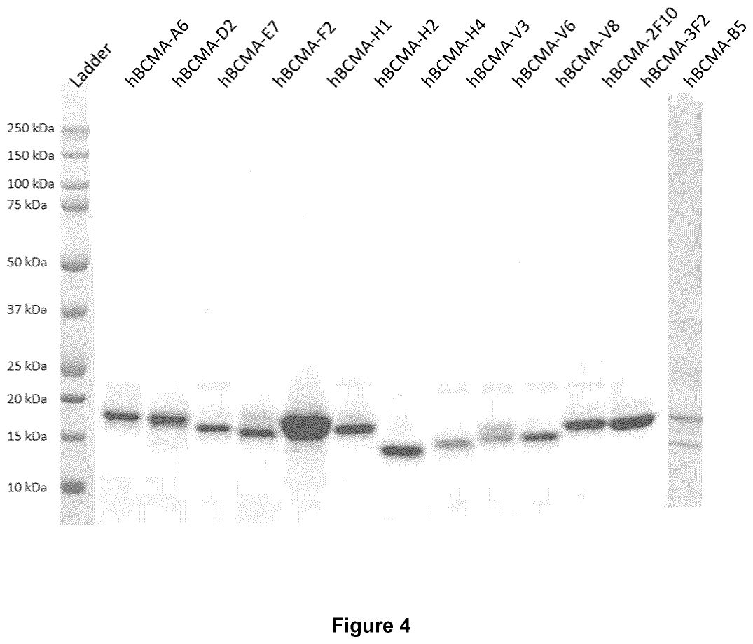

The gene synthesis and sub-cloning was performed by TWIST Bioscience (USA) and the plasmid DNA were transformed into BL21(DE3) E. coli for protein expression. The presence of a Histidine tag and biotinylation signal sequence (Avitag™) in the pMRO vector allows facile purification by IMAC column as well as specific addition of a biotin moiety at the VHH C-terminal. The single biotin addition facilitates VHH detection in future epitope mapping and other cell-based assays. The IMAC-purified VHH proteins were run on a SDS-PAGE (FIG. 4). As shown, the VHH antibodies showed an expected molecular weight of around 15-17 kDa. The VHH proteins for two VHHs (2C3 and 4D1) are not shown in this figure as they are quite highly similar to VHH-E7 and VHH-H2.

The state of aggregation of the purified protein was checked by size exclusion chromatography and as expected all were non-aggregating monomers. The reactivity of the individual VHH protein was also confirmed by ELISA in which rabbit anti-His₆ antibody conjugated to HRP was used for the detection of VHH binding to the immobilized BCMA-ECD (data not shown).

The monomeric fraction of all 15 VHHs were used for SPR experiment where the human BCMA-ECD or mouse BCMA was immobilized onto the CM5 dextran chip and various VHH concentration (20-500 nM) were passed over the sensor chip. SPR analysis revealed all 15 VHHs specifically bound BCMA-ECD with equilibrium constants ranging from 4 nM for hBCMA-A6 to 0.14 pM for hBCMA-E7. All of the data collected fit a 1:1 binding model except BCMA-A3 VHH which did not generate reliable binding data.

For epitope binning of anti-BCMA VHHs, co-injection SPR experiments were performed with pairs of VHHs in both orientations to determine if antibodies could bind BCMA-ECD simultaneously. If there is an increase in response upon co-injection of any two VHHs, this will indicate that binding of the first VHH (at saturation concentration) does not hinder the binding of the second one and, therefore, these antibodies recognize independent epitopes. However, if there is a minor change in response upon co-injection of two VHHs, this will indicate that the two VHHs could not bind simultaneously to the same region and, therefore, they recognize overlapping/identical epitopes. The co-injection SPR experiments were performed for 4 selected anti-BCMA VHHs and identified no VHH binding to distinct epitope as the ECD-BCMA domain is only 54 aa and there may not be sufficient spacing for the two VHHs to bind simultaneously.

FIG. 4 depicts the SDS-PAGE of 13 anti-BCMA VHH antibodies expressed in BL21(DE3) E. coli and purified by IMAC. The purified proteins showed expected molecular weight of 15-17 kDa and there was no sign of degradation in all protein samples. There is an additional smaller band in BCMA-B5 which was excluded by size exclusion chromatography when measuring its binding affinity. VHH #2 (203) and VHH #4 (401) yielded similar protein bands on SOS-PAGE (data not shown).

Table 1 depicts the amino acid sequences of all 15 VHHs. The CDR (underlined) and Framework regions are numbered according to IMOT numbering system.

FIGS. 5A and 56 together depicts the alignment of amino acid sequences of 15 VHHs.

Tables 3A and 36 depicts the measured affinities of all 15 VHHs as described in the text. The affinities data range from 0.14 pM (hBCMA-E7) to 4 nM (hBCMA-A6).

TABLE 3A
Measured Affinities for VHHs for human BCMA ECD

| Clone No.-Bin | Old name | Target | ka(1/Ms) | kd (1/s) | KD (M) |
|---|---|---|---|---|---|
| VHH # 1-B1 | hBCMA-E7 | hBCMA-ECD-FC [symbol] | 3.88E+06 | 1.79E−04 | 4.62E−11 |
| [symbol] | [symbol] | [symbol] | [symbol] | [symbol] | [symbol] |
| VHH # [symbol] -B1 | hBCMA-H2 | hBCMA-ECD-FC [symbol] | 4.28E+06 | 3.43E−04 | 8.01E−11 |
| [symbol] | [symbol] | [symbol] | [symbol] | [symbol] | [symbol] |
| VHH # [symbol] -B1 | hBCMA-VcMRo3 (V3) | hBCMA-ECD-FC [symbol] | 4.24E+05 | 2.39E−04 | 5.64E−10 |
| VHH # [symbol] -B1 | hBCMA-2F10 | hBCMA-ECD-FC [symbol] | 4.22E+06 | 9.10E−04 | 2.16E−10 |
| VHH # [symbol] -B1 | hBCMA-3F2 | hBCMA-ECD-FC [symbol] | 4.01E+06 | 2.53E−03 | 6.31E−10 |
| VHH # [symbol] -B2 | hBCMA-B5 | hBCMA-ECD-FC [symbol] | 1.08E+07 | 2.37E−03 | 2.20E−10 |
| VHH # [symbol] -B2 | hBCMA-H4 | hBCMA-ECD-FC [symbol] | 1.34E+06 | 5.09E−04 | 3.79E−10 |
| VHH # [symbol] -B2 | hBCMA-H1 | hBCMA-ECD-FC [symbol] | 3.05E+06 | 4.62E−04 | 1.52E−10 |
| VHH # [symbol] -B3 | hBCMA-F2 | hBCMA-ECD-FC [symbol] | 5.11E+06 | 3.49E−04 | 6.83E−11 |
| VHH # [symbol] -B3 | hBCMA-A6 | hBCMA-ECD-FC [symbol] | 2.36E+06 | 1.09E−02 | 4.62E−09 |
| VHH # [symbol] -B3 | hBCMA-VcMRo1(V1/V6) | hBCMA-ECD-FC [symbol] | 8.67E+05 | 2.62E−04 | 3.03E−10 |
| VHH # [symbol] -B3 | hBCMA-D2 | hBCMA-ECD-FC [symbol] | 2.42E+06 | 9.78E−04 | 4.03E−10 |
| VHH # [symbol] -B4 | hBCMA-VcMRo8(VF7/VF8) | hBCMA-ECD-FC [symbol] | 1.48E+07 | 1.25E−03 | 8.46E−11 |

[symbol]

indicates data missing or illegible when filed

TABLE 3B
Measured Affinities for VHHs murine BCMA ECD

| Clone No.-Bin | Old name | Target | ka(1/Ms) | kd (1/s) | KD (M) |
|---|---|---|---|---|---|
| VHH # 1-B1 | hBCMA-E7 | mBCMA-ECD-FC [symbol] | 7.62E+05 | 5.141E−02 | 6.75E−8 |
| [symbol] | [symbol] | [symbol] | [symbol] | [symbol] | [symbol] |

[symbol]

indicates data missing or illegible when filed

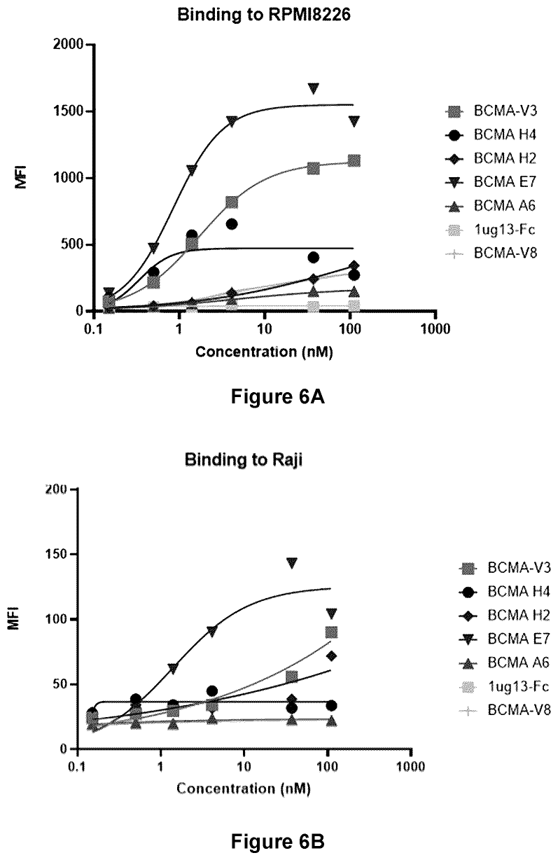

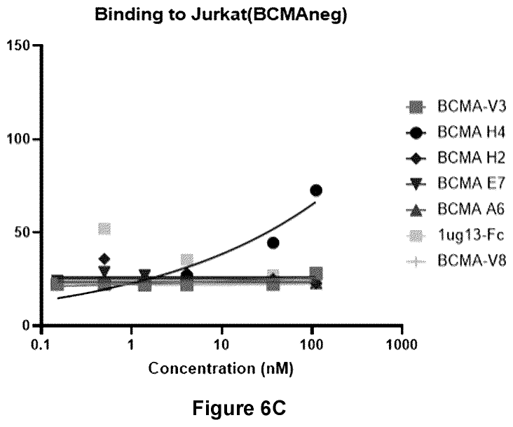

FIG. 6 depicts binding of anti-BCMA VHH to tumor cell lines with high (RPM18226), low (Raji) or no (Jurkat) BCMA expressing cells.

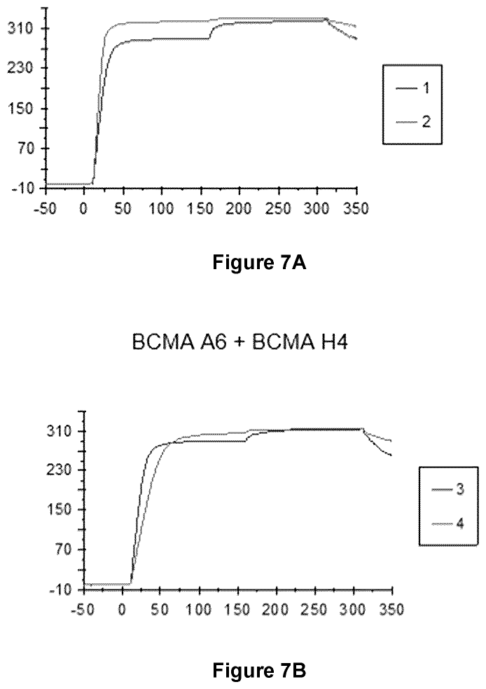

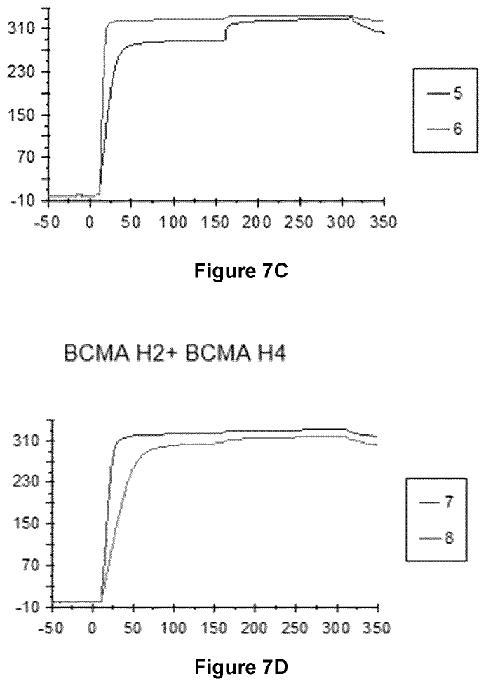

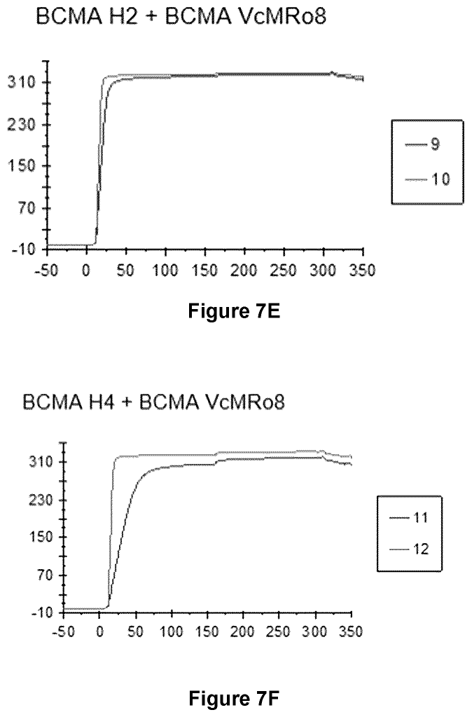

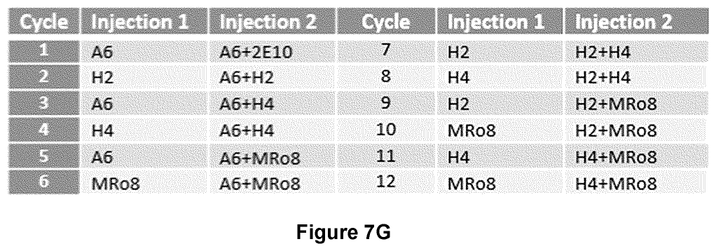

FIG. 7 depicts competitive binding data by SPR.

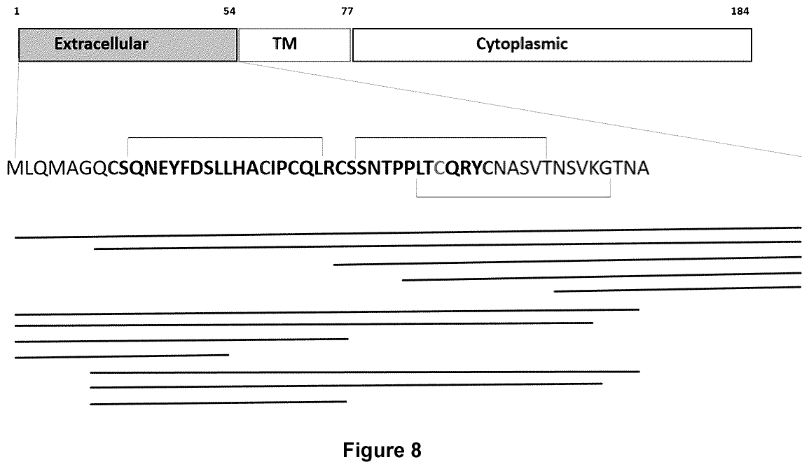

FIG. 8 depicts the sequence of the ecto-domain of the hBCMA and the positions of the disulfide bonds, and the yeast surface display constructs expressing the various BCMA fragments used cell ELISA for epitope mapping of the sdAbs presented in Example 1.

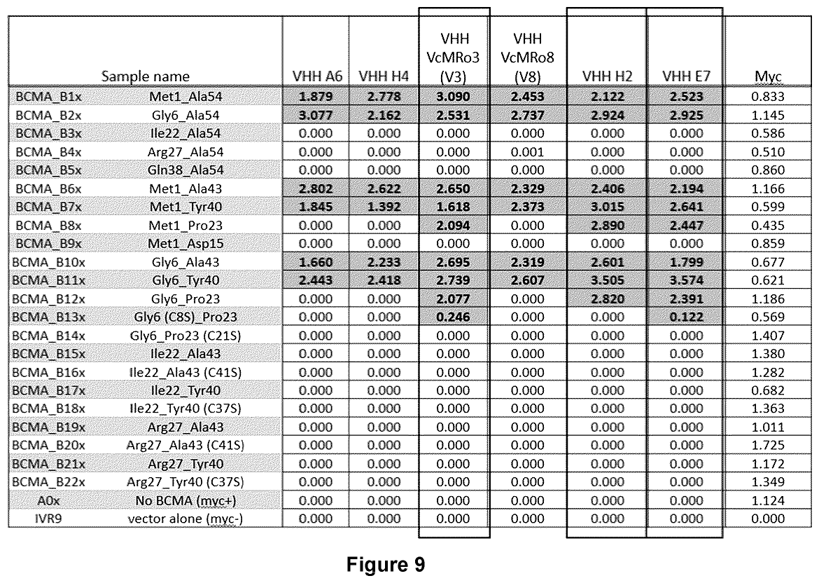

FIG. 9 depicts the yeast cell ELISA measured binding of selected sdAbs presented in Example 1 against yeast surface displayed various hBCMA ecto-domain fragments as indicated. The assays were performed as described in the Materials and Method, and OD₄₅₀ was measured. Note that OD₄₅₀ reads equal or less than 0.100 were scored as zero for clarity.

Two epitopes (I and II) were recognized differentially by the sdAbs, epitope I located in fragment encompassing Gly6-Pro23, recognized by VHH-E7, VHH-H2 and VcMRo3; VHH-A6, VHH-H4 and VcMRo8 bound epitope II located in fragment Gly6-Tyr40 of hBCMA. The two epitopes have been mapped onto the structure (PDB:2KN1) of BCMA extracellular domain.

### Discussion

Anti-BCMA-ECD VHHs were expressed in E. coli and the proteins were purified and biotinylated. The antibodies showed non-aggregating and monomeric behaviors as determined by size exclusion chromatography.

The binding kinetics of 15 VHHs were determined by SPR and the antibodies showed specific binding to human BCMA-ECD with affinities ranging from low nM to sub pM (4 nM for hBCMA-A6 to 0.14 pM for hBCMA-E7). This diverse set of affinities allows us to study the effect of affinity in productivity of CAR-T construct. Epitope binning of 4 representative out of 15 VHHs based on their sequence identities by SPR indicates that all the VHHs bind to the same or overlapping epitopes on the 54 aa extracellular domain of the BCMA. The possibility of competing VHHs to neighboring region could not be ruled out by SPR epitope binning. However, using a collection of various fragments human BCMA ecto-domain expressed as yeast surface display, it was possible to physically map out two epitopes that are differentially required for the binding of sdAbs presented in Sample 1. The two epitopes are physically different but overlapped at their N-terminal portion. It appears that, at current resolution, the whole epitope I is part of the epitope II. The physical epitope mapping data explains the earlier observation of the competition SPR data, as the anti-BCMA sdAbs presented in Sample 1 have overlapping epitopes and either direct competition or steric hindrance would be scored as functional competition. Given the relatively small landscape of the antigen, epitopes can be “crowded”. For example, it appears that binding of e.g., BCMA-H4 sdAb to Gly6-Try-40 region prevented any further binding of BCMA-H2 sdAb to Gly6-Pro23 region, and vice versa.

### Example 3: CAR-T In Vitro Testing

### Introduction

After identifying novel BCMA-binding single domain antibody (sdAb) sequences described above, it was desired to test their activity within the context of chimeric antigen receptor (CAR) molecules which can be used to redirect human T cell responses towards cells bearing specific surface antigens. Thus, using high throughput techniques previously described (Bloemberg 2020) novel BCMA-sdAb targeted CAR constructs were generated and tested their relative T cell activating activity via various assays described below.

### Materials and Methods

Single domain antibody antigen binding sequences (ABD) were transferred to a modular CAR plasmid backbone (e.g., see SEQ ID NO: 68) containing restriction sites to allow efficient recombination wherein the antigen binding domain could be removed and replaced with the novel BCMA-sdAb antigen binding domain (ABD) sequences. Specific CAR design used was as follows: Human CD28 signal peptide (SEQ ID NO: 69), ABD (any one of SEQ ID NOs: 40 to 58), flexible linker domain (SEQ ID NO: 70), human CD8 hinge domain (SEQ ID NO: 71), human CD28 transmembrane domain (SEQ ID NO: 72), human 4-1BB signal transduction domain (SEQ ID NO: 73), and human CD3-zeta signal transduction domain (SEQ ID NO: 74). Control constructs were also generated using sequences derived from previously demonstrated CD19-specific CAR sequence.

For some sdAbs, sequence changes were made. The N-terminal region of sdAb E7 was changed from QVKLEE to QVQLVE (see SEQ ID NO: 54), which led to stability improvements. For similar reasons, the N-terminal region of sdAb H2 was changed from QVQLVE to QVKQEE (see SEQ ID NO: 55).

With use of degenerate primers, the third position of the FR4 region could switch from L to Q and vice versa.

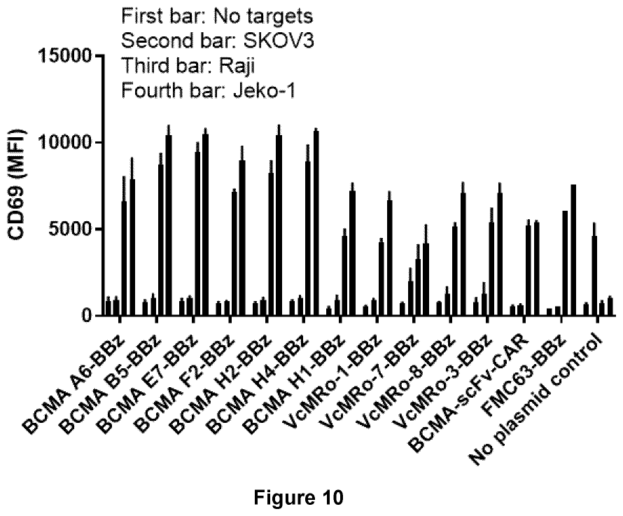

Novel BCMA-targeting CAR constructs were then tested for activity in an immortalized human T cell line (Jurkat) similarly as described in Bloemberg 2020. In brief, plasmids were electroporated into Jurkat T cells and allowed to recover for several hours. Jurkat-CAR cells were then mixed at varying doses with target cell lines exhibiting varying expression levels of human BCMA. Target cell lines with varying BCMA expression of (BCMA+ Raji or Jeko-1; BCMA-negative SKOV3) were utilized for this study to confirm CAR activation activity in Jurkat cells. In order to quantitate CAR-mediated Jurkat cell activation, expression of CD69 was measured using specific antibody staining and flow cytometry. Using expression of GFP-marker to gate CAR-expressing cells, the level of T cell activation as determined using the CD69-surface marker was clearly elevated in various Jurkat cells expressing various BCMA-sdAb targeted CAR constructs when cells were placed in co-culture with BCMA expressing Ramos cells but not with BCMA-negative cells (FIG. 10).

Following this CAR-J testing, several BCMA-CAR constructs were selected for testing in primary human T cells. To accomplish this, lentivirus was prepared through co-transfection of CAR plasmids with lentiviral packaging cell lines. Lentiviral particles in the cell supernatant were collected and concentrated using ultracentrifugation. Primary human T cells were then isolated from a donor blood samples using magnetic bead separation and polyclonally activated using anti-CD3 and anti-CD28 beads. Activated human T cells were then transduced with concentrated lentivirus containing various BCMA-targeted CAR constructs at pre-determined multiplicity of infection. Following viral transduction, cells were confirmed to express CAR using flow cytometric analysis for GFP-marker. Virally transduced T cells (CAR-T cells) were then expanded for 9 days before examination for CAR activity.

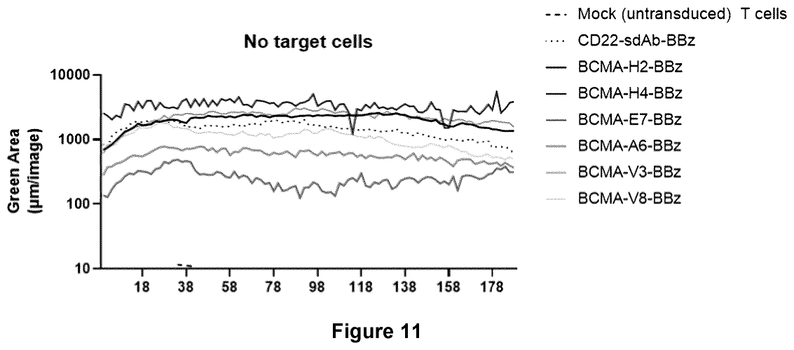

To examine CAR activity in virally transduced CAR-T cells a number of assays were utilized. Firstly cells were placed without additional stimulation in controlled cell culture conditions and examined for non-specific cellular expansion over an additional 6 days via live microscopy using an IncuCyte® S3 device (Sartorius, USA). Total cell count was determined using automated cell counting. Primary human T cells stably transduced with various BCMA-sdAb targeted CAR constructs did not show significant cell expansion when left in unstimulated conditions between day 9 and 15 post-polyclonal activation (FIG. 11). These results indicate that BCMA-sdAb targeted CAR constructs tested do not confer target-independent tonic T cell activation to primary human T cells.

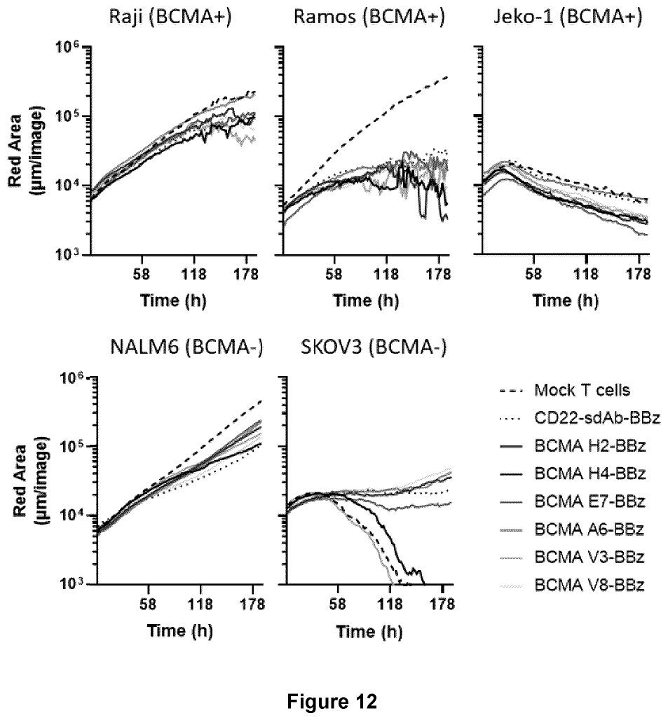

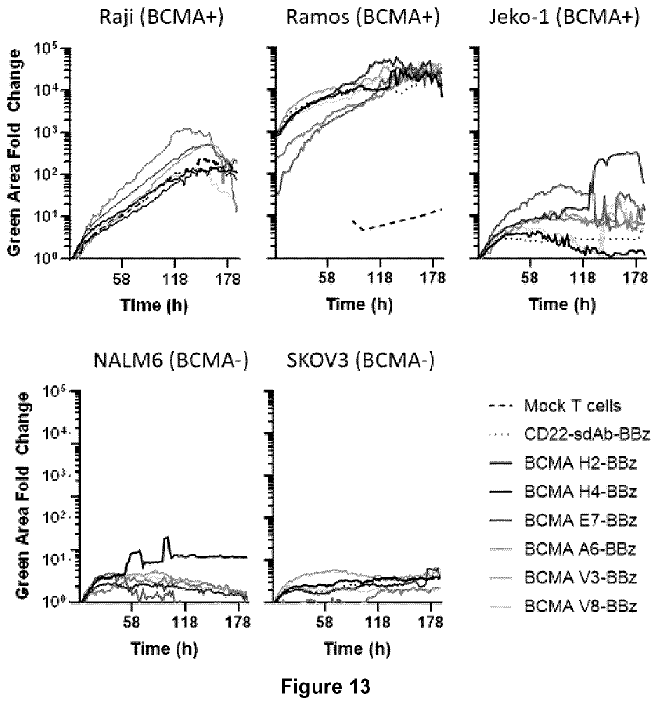

Following this, primary CAR-T cells were tested for antigen specific activation and target cell killing in response to cells with and without BCMA expression (BCMA-positive: Raji, Ramos, Jeko-1; BCMA-negative: NALM6, SKOV3). CAR-T cells were placed in co-culture with various target cells expressing a red-fluorescent protein tag, NucLight™-Lentivirus (Sartorius, USA), and monitored for 6 days using the IncuCyte S3 live microscopy device. CAR-T mediated target cell growth repression occurred with all BCMA-positive target cell lines but was most apparent with Ramos (FIG. 12; top 3 panels). Target growth repression for BCMA-negative targets was variable across constructs tested (FIG. 12; bottom 2 panels). Examining the number of GFP-labelled CAR-T cells, all BCMA CAR constructs showed clear expansion of GFP+ cells in response to BCMA+ cell lines (Raji, Ramos), with lower responses against BCMA-low Jeko-1 cells, and no apparent CAR-T expansion in response to a BCMA-negative cell line (NALM6, SKOV3) demonstrating antigen-specific activation and expansion (FIG. 13).

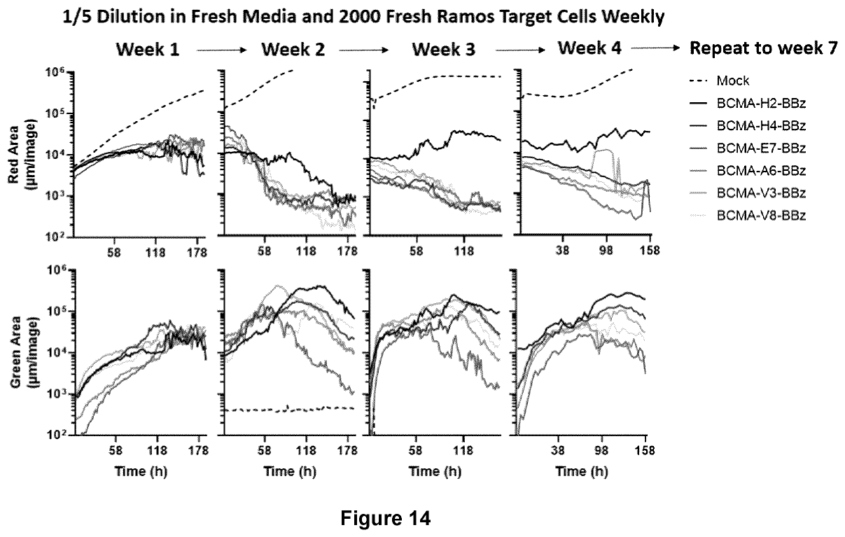

Next, experiments were undertaken to demonstrate serial killing capacity in novel BCMA sdAb targeted primary CAR-T cells. As described above, CAR-T cells were generated from donor blood derived T cells using lentiviral transduction and expanded for 9 days in cell culture. CAR-T cells were then placed in co-culture with fluorescently labelled BCMA expressing target cells (Ramos). After 1 week co-cultures were diluted with fresh media (1 in 5 dilution with cytokine supplemented media) and fresh target cells were also added to the cultures at a similar number to the initial target dose. Subsequently, assessment of target cell expansion (red fluorescence) and CAR-T expansion (green fluorescence) demonstrates sustained ability of BCMA-CAR-T cells to respond to target cells over repeated challenged for 4 weeks (FIG. 14). Similar long-term repeated co-cultures were maintained for target cells with varying BCMA-expression (data not shown).

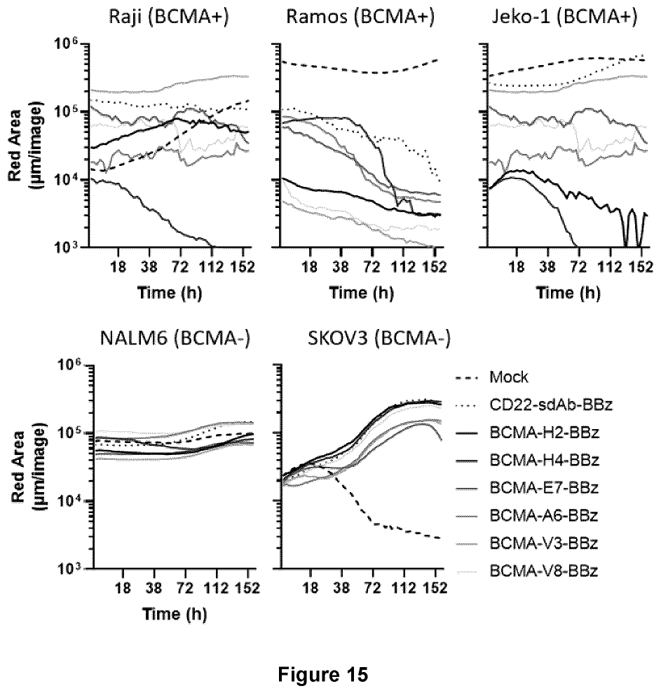

Long-term co-culture challenges with target cells with varying BCMA-expression were repeated for 5 weeks, followed by 1 week of resting in media without additional target challenge. Week 6 rested co-cultures were then diluted with fresh media and re-challenged with additional target cells as described above. Monitoring of red fluorescent target cell growth within these week 7 co-cultures demonstrates varying capacity of BCMA-CAR-T cells to repress BCMA-positive target cells (FIG. 15, top 3 panels). Response to Ranking based on these results reveals highest target repression activity for H4, then H2, then A6, which is equivalent to E7, V3, and V8. No CAR-T cells showed responses to BCMA-negative NALM6 target cells after 7-weeks in co-culture, and only untransduced (mock) T cells showed repression of SKOV3 cells (FIG. 15, bottom 2 panels).

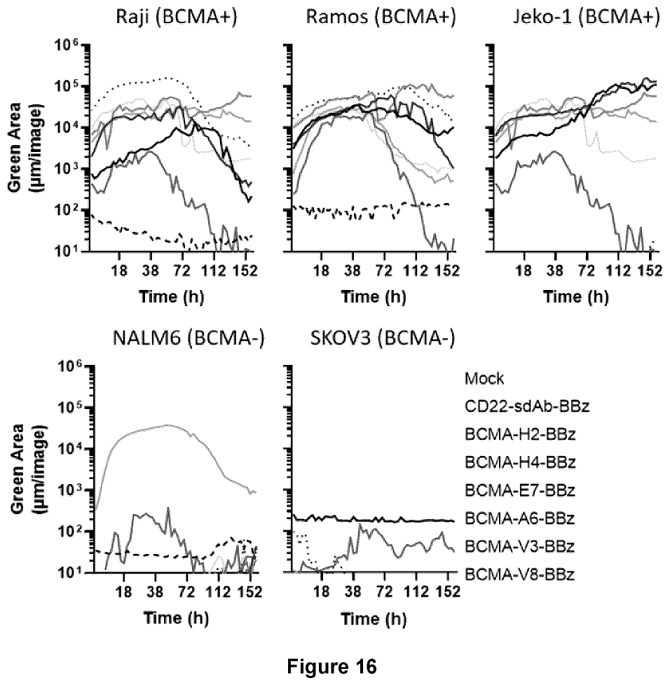

Examining GFP-marked CAR-T expansion in week 7 co-cultures, results show very good expansion of all CAR-T constructs in co-culture with BCMA-positive target cells (FIG. 16 top 3 panels). Only BCMA-V3-BBz construct showed a response to BCMA-negative NALM6 cells (FIG. 16, bottom left panel), and no CAR-T cells showed response to BCMA-negative SKOV3 cells (FIG. 16, bottom right panel). Ranking CAR-T constructs in this assay shows similar responses for H4, H2, and A6 CAR constructs, with lower responses for V3, then V8, then E7 CAR constructs. Overall long-term co-culture results demonstrates that BCMA-CAR-T cells maintain potent antigen-specific responsiveness even after several weeks of repeated exposure to target cells.

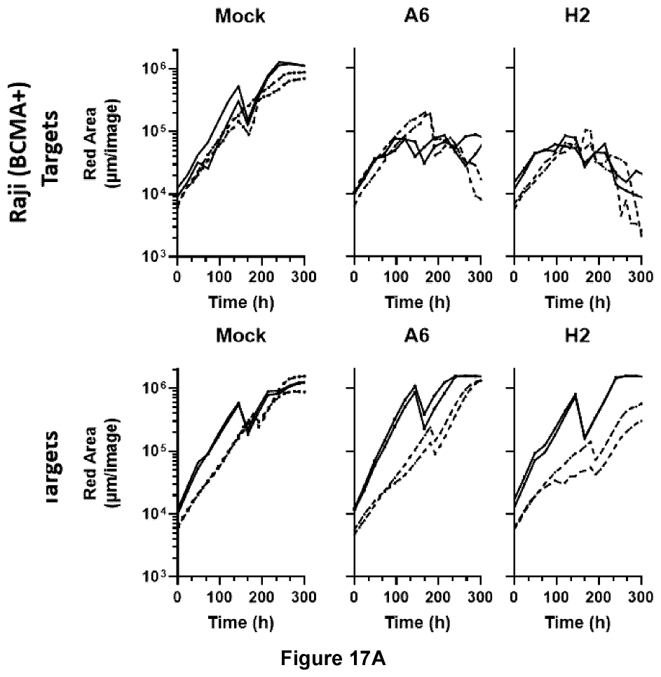

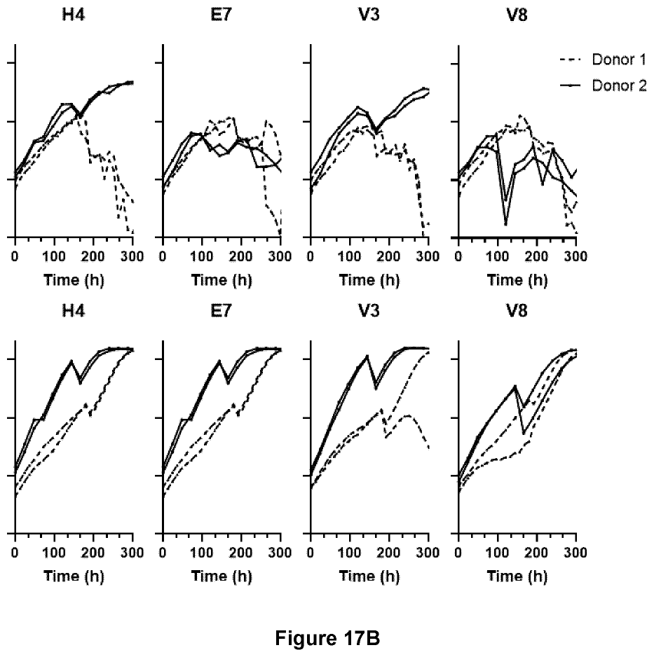

To investigate inter-donor variability with the novel BCMA-sdAb targeted CAR constructs additional CAR-T cells were generated as described above from two different donor blood samples. In this experiment, the novel CAR-T constructs were introduced into polyclonally stimulated blood derived T cells from two healthy donors using lentiviral transduction as described above. CAR-T cells were then placed in co-culture with BCMA-expressing target cells (Raji) or BCMA-negative target cells (NALM6) and examined for tumour cell growth repression (FIG. 17A) and CAR-T cell expansion (FIG. 17B). After 7 days, co-cultures were diluted with fresh media and additional target cells were added as described above. Results demonstrate that BCMA-CAR constructs shows consistent and similar response to BCMA-positive target cells and low responses to BCMA-negative target cells with CAR-T cells generated from varying donors.

Next, it was desired to test whether these BCMA-CAR constructs could also show antigen specific CAR activity when expressed within NK-cells rather than T cells. Thus, similarly as described above for T cells, the immortalized human NK92 cells were transduced with various BCMA-CAR constructs. NK92-CAR cells were then co-cultured at varying ratios with BCMA-positive target cells (RPM18226 or Raji) or BCMA-negative target cells (NALM6). Surface expression of CD107a degranulation marker and increased intracellular expression of interferon-gamma demonstrates BCMA-specific responsiveness in all 3 constructs tested here. Overall results indicate that BCMA-CAR constructs can have antigen-specific responsiveness activity in human NK cells.

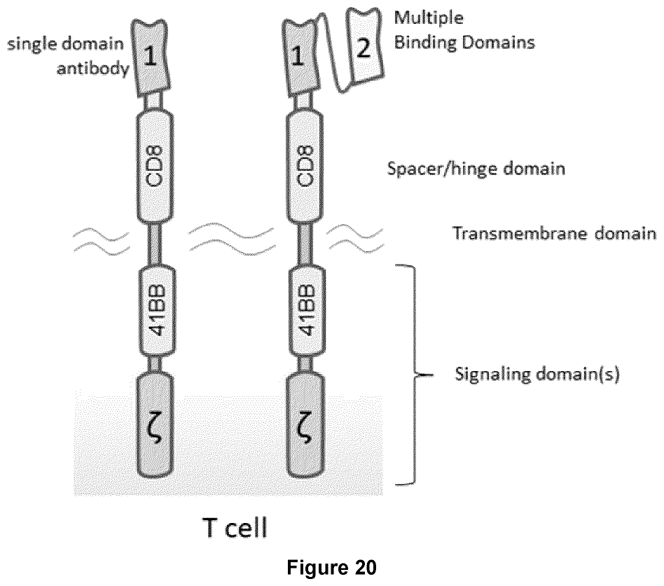

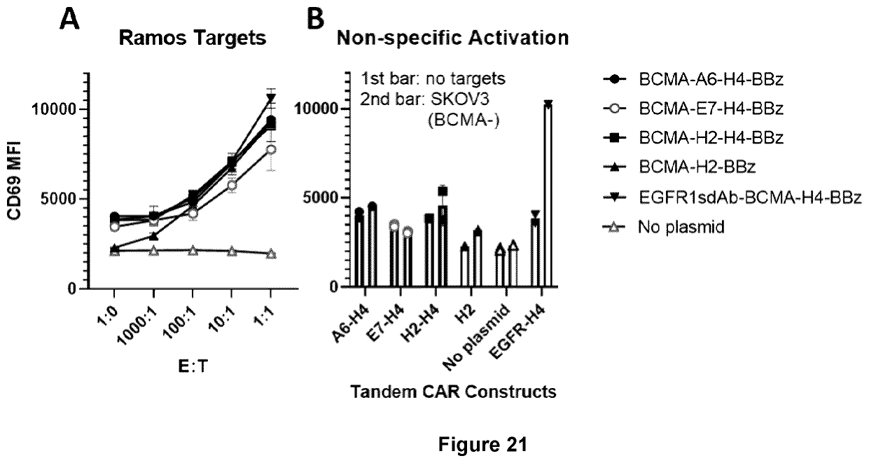

Lastly, it was investigated whether the BCMA-single domain antibody targeting moieties tested here could also have functionality when combined in multi-binder or multi-antigen binding constructs. SEQ ID NO: 77 is an example multi-binder comprising sdAbs A6 and H4. In contrast to single binders (FIG. 20 left), multi-binder CAR constructs can contain two or more binding elements (FIG. 20 right). In order to test the functionality of such constructs, synthetic DNA was generated expressing multiple BCMA single domain antibody sequences (SEQ ID NO: 79 provides an exemplary amino acid sequence for BCMA-A6-H4-BBz CAR), or a BCMA-sdAb and EGFR-targeted single domain antibody sequence connected by a G4S linker sequence within the modular CAR plasmid backbone. These plasmids were then transiently expressed in Jurkat cells using electroporation. Jurkat-CAR cells were then co-cultures with BCMA-positive Ramos cells at varying effector:target ratios, which induced clear activation of all BCMA-multi-binder constructs similar to a single binder construct as measured by CAR-T cells CD69 expression (FIG. 21, panel A). Examining the level of Jurkat-CAR cell activation in the absence of target cells of with BCMA-negative target cells shows relatively low tonic signaling for BCMA-multi-binder constructs (FIG. 21, panel B). As an example of multi-antigen targeting with a similar CAR structure, a CAR with both a BCMA and EGFR sdAb binding elements was tested. This construct showed good response to both BCMA-positive Ramos cells and EGFR-positive SKOV3 cells (FIG. 21, panels A B). Overall, these results demonstrate that BCMA-sdAbs tested here maintain strong antigen response activity when combined in multi-binder CAR constructs.

### Results

FIG. 10 depicts the results of CAR-Jurkat assay wherein Jurkat cells were transiently electroporated with varying CAR plasmids and cultured alone or in co-culture with BCMA-positive (Ramos or Jeko-1) or BCMA-negative (SKOV3) cell lines. The level of T cell activation was measured using human CD69-specific antibody staining and flow cytometry. Graphs depict the mean fluorescent intensity for CD69-staining for each single domain antibody targeted CAR constructs performed in a single experiment in duplicate, either in culture with no target cells (first bar), BCMA negative SKOV3 target cells (second bar), or BCMA positive Ramos or Jeko-1 target cells (third and fourth bar respectively). Error bars show the standard error of the mean for duplicate wells. Results demonstrate antigen-specific response with all of the novel BCMA CAR constructs tested.

FIG. 11 depicts the results of CAR-T tonic activation assay wherein primary donor blood derived T cells were transduced with varying CAR constructs and examined for target-independent expansion. Mock refers to donor derived T cells exposed to similar treatment conditions in the absence of any CAR-expressing lentivirus. As described in methods, CAR-T cells were examined between day 9 and 15 post-polyclonal activation for proliferation in cell culture via live microscopy. Graphs depict the fold change in GFP-marked CAR-T cell number relative to number of cells the start of this assay as determined using automated cell counting. Results demonstrate a lack of antigen-independent T cell expansion in those CAR constructs tested.

FIG. 12 depicts the results of CAR-T target growth repression assay performed using donor blood derived T cells transduced with varying BCMA-single domain antibody or CD22-specific comparator CAR constructs. Mock refers to unmodified donor derived T cells without CAR expression exposed to similar treatment conditions. As described above, red fluorescent protein (mKate2) marked target cells with varying BCMA expression (BCMA-pos: Raji, Ramos, Jeko-1; BCMA-neg: NALM6 and SKOV3) were examined via live fluorescent microscopy for target cell proliferation when in co-culture with BCMA+ target cells. Graphs depict the total red fluorescent protein marked target cells as determined using automated counting. Results demonstrate specific repression of BCMA-expressing target cells by BCMA-CAR-T cells, wherein all BCMA constructs tested show significant expansion and thus were all selected as hits for downstream testing.

FIG. 13 depicts the results of CAR-T target-specific activation assay performed using donor blood derived T cells transduced with varying BCMA-single domain antibody or CD22-specific comparator CAR constructs. Mock refers to unmodified donor derived T cells without CAR expression exposed to similar treatment conditions. As described above, GFP-marked CAR-T cells were examined via live fluorescent microscopy between day 9 and 15 post-polyclonal activation for proliferation in co-culture with target cells with varying BCMA-expression (BCMA-pos: Raji, Ramos, Jeko-1; BCMA-neg: NALM6 and SKOV3). Graphs depict the total green fluorescent protein signal as determined using automated counting. Results demonstrate specific expansion of CAR-T cells in response to BCMA-expressing target cells, wherein all BCMA constructs tested show significant expansion and thus were all selected as hits for downstream testing.

FIG. 14 depicts the results of CAR-T target-specific serial killing assay performed using long-term co-culture assay of donor blood derived CAR-T cells transduced with varying BCMA-single domain antibody or comparator CD22-CAR constructs generated as described above. Mock refers to unmodified donor derived T cells without CAR expression exposed to similar treatment conditions. CAR-T or Mock-T cells were placed in co-culture with red-fluorescent protein expressing BCMA+ target cells and examined for target growth (top panel) or CAR-T cell growth via automated cell counting (bottom panel). Six days post initial challenge, cells were split 1 in 5 in fresh media and challenged with 2000 additional target cells. This procedure was repeated for a total of 7 weeks to assess the long term killing capacity of BCMA CAR constructs (only data to 4 weeks is shown). Graphs shown depict co-culture with BCMA+ Ramos cells, but similar experiments were performed with targets with varying BCMA expression (BCMA-pos: Raji, Ramos, Jeko-1; BCMA-neg: NALM6 and SKOV3).

FIG. 15 depicts the results of CAR-T co-culture assay performed over week 7 of the long-term co-culture assay. Donor blood derived CAR-T cells transduced with varying BCMA-single domain antibody, comparator CD22-CAR constructs, or with no CAR construct (mock) as described above. Co-cultures were examined as described above for red fluorescent protein (mKate2) marked target cell growth in co-cultures with varying BCMA expression (BCMA-pos: Raji, Ramos, Jeko-1; BCMA-neg: NALM6 and SKOV3). Graphs depict the total red fluorescent protein found in wells as determined using automated counting. Results stratify BCMA-specific CAR constructs based on long term repression: with H4 showing the highest activity followed by H2, then by A6 which was approximately equivalent to E7, V3, and V8.

FIG. 16 depicts the results of CAR-T co-culture assay performed over week 7 of the long-term co-culture assay. Donor blood derived CAR-T cells transduced with varying BCMA-single domain antibody, comparator CD22-CAR constructs, or with no CAR construct (mock) as described above. Co-cultures were examined as described above for green fluorescent protein (GFP) marked CAR-T cell growth in co-cultures with varying BCMA expression (BCMA-pos: Raji, Ramos, Jeko-1; BCMA-neg: NALM6 and SKOV3). Graphs depict the total green fluorescent protein signal found in wells as determined using automated counting. Results stratify BCMA-specific CAR constructs based on CAR-T expansion: with H2, H4, and A6 showing the highest activity, followed by V3, then by V8 then by E7 CAR construct.

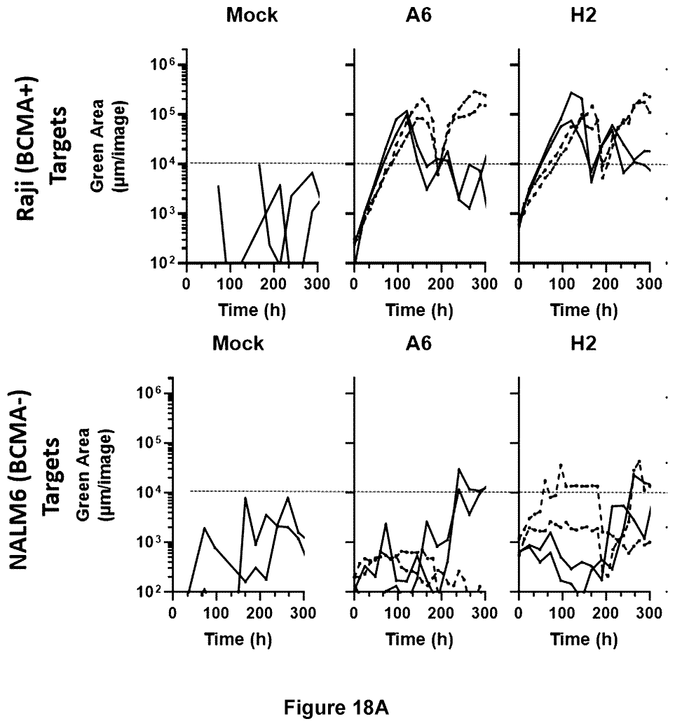

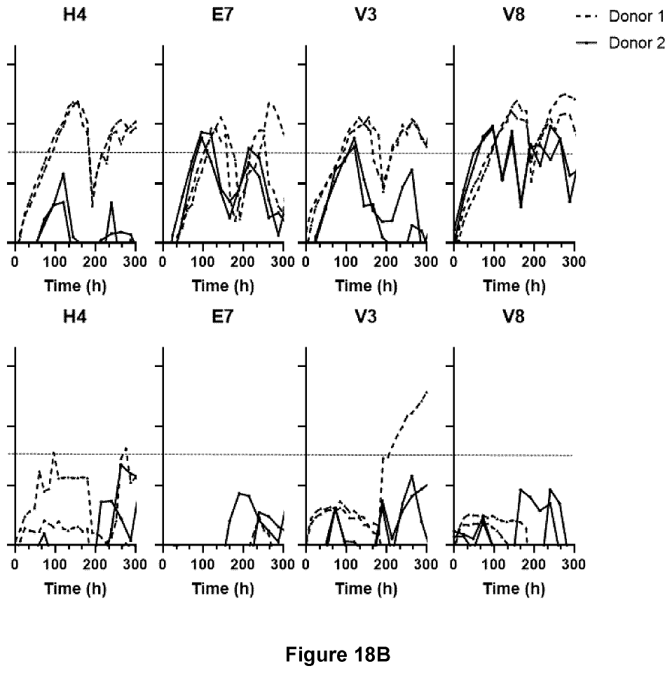

FIGS. 17A, 17B, 18A, and 18B depict results of consistency analysis and comparison with un-transduced T cells (mock) and BCMA-sdAb targeted CAR-transduced T cells generated from 2 separate donors as described above. CAR-T cells were placed in duplicate wells in co-culture with BCMA+ target cells (Raji) or BCMA− targets (NALM6) and examined via live fluorescent microscopy. Graphs depict the total red fluorescent protein (NucLight) signal from marked target cells (FIG. 17A). FIGS. 18A and 18B depict total green fluorescent protein signal from CAR cells as determined using automated counting. Results demonstrate intra- and inter-donor consistency for BCMA CAR-T specific repression of growth of BCMA+ target cells and target-induced expansion of most of the BCMA CAR-T cells constructs tested.

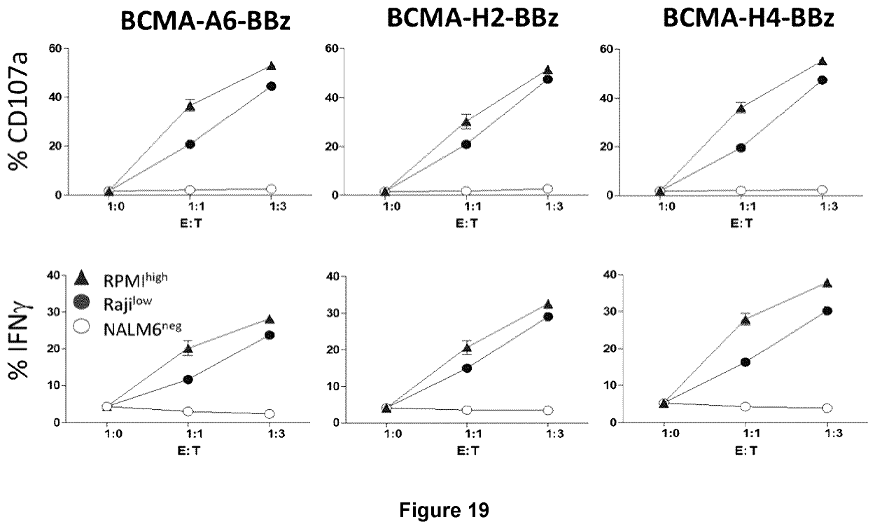

FIG. 19 depicts the results of an assay to test the activity of varying BCMA-specific CAR-T constructs within NK-cells. In brief, the human immortalized NK cell line (NK92) was transduced with lentiviral vectors encoding BCMA-targeted CAR constructs (BCMA-sdAb-BBz) similarly as described above for T cells. BCMA-CAR-NK92 cells were then placed in co-culture BCMA-positive target cells (RPM18226 or Raji) or BCMA-negative target cells (NALM6). After several hours in co-culture, response of NK92 cells were examined via antibody staining for NK degranulation marker CD107a, or intracellular cytokine staining for interferon-gamma via flow cytometry. Results demonstrate BCMA-specific responsiveness of BCMA-CAR-NK92 cells.

FIG. 20 depicts the molecular structure of a single-binder (left) or multi-binder (right) BCMA-specific chimeric antigen receptor; for multi-binder CAR constructs a BCMA-sdAb sequence at the 5′ end of a CAR DNA construct is followed by a linker sequence which can be of varying composition, followed by another sdAb sequence which can be the same of different from the first sdAb sequence included in the sequence, the followed by a similar structure to other CAR molecules [hinge domain, transmembrane domain, signaling domain(s)]. A similar molecule structure can also be used to generate multi-antigen binding CAR constructs wherein a BCMA-sdAb sequence is followed by a linker and then an alternate sdAb sequence targeting a different antigen

FIG. 21 depicts the results of Jurkat cell CAR activation activity assay wherein CAR plasmids with varying single or multi-binder formats were electroporated into Jurkat cells, which were then placed in co-cultures containing BCMA-positive (Ramos; left), without target cells or with BCMA-negative (SKOV3) target cells (right). Graphs depict the average CD69-specific antibody staining of Jurkat cells as measured by flow cytometry after overnight incubation of co-cultures. Error bars present the standard error of the mean over 2 duplicate co-culture wells. Results demonstrate similar BCMA-antigen specific activation of T cells expressing CAR molecules with single BCMA binding elements, multiple BCMA binding elements, or BCMA and an EGFR-targeted binding element.

### Discussion

Overall these results exemplify that BCMA-specific single domain binders can generate strong antigen-driven T cell activation signaling which can drive target cell killing, target serial killing, long-term tumour cell growth repression, and CAR-T expansion even after repeated challenges over an extended period of time. Data is also provided demonstrating that these BCMA-specific CAR constructs produce strong antigen-specific response in both T and NK cells. While few lead molecules were identified in the exemplary data provided here, molecular optimization may be performed with additional BCMA-specific single domain antibody sequences in order to generate highly functional CAR molecules. As an example of such molecular optimization, data was provide demonstrating that when expressed in multi-binder and/or multi-antigen targeting CAR format, BCMA-constructs maintain strong antigen-specific responsiveness. In addition, combining multiple BCMA-specific single domain antibody sequences in a single molecule may be an effective strategy to increase target-specific CAR activating activity.

### Example 4: CAR-T In Vivo Testing

### Introduction

To further confirm the anti-tumor effect of BCMA-binding single domain CAR-T cells in vivo, a xenograft model was established by intravenously inoculating Ramos tumor cells expressing firefly luciferase as a reporter into NOD/SCID/IL2r-gamma-chain^{null} (NSG) mice prior to infusion of BCMA-targeting single domain antibody CAR-T cells.

### Materials and Methods

For in vivo studies, luciferase-expressing cell lines were generated by stably transducing wild-type tumor lines with lentiviral vector encoding firefly luciferase (FLUC) followed by selection of luciferase-positive cells using puromycin resistance as a selection marker. Ramos-FLUC was maintained in RPMI 1640 supplemented with 10% heat inactivated fetal bovine serum and 2 mM L-glutamine and 1 mM sodium pyruvate. All cell culture reagent were purchased from Gibco. The cell line were confirmed for the absence of mycoplasma contamination PCR.

Female NOD/SCID/IL2Ry^{-/-} (NSG) mice, 6-8 weeks of age, were obtained from Jackson Laboratories and maintained at the Animal Care Facility at the National Research Council of Canada. The mice were housed in pathogen-free individually ventilated cages in a barrier system under conditions. Animals had access to certified rodent diet and sterilized water was given via water bottles. NSG mice lack mature T cells, B cells and natural killer cells; thus, they are better than nu/nu mice for the study. Eight-week-old NSG mice were injected with 5×10⁴ Ramos-FLUC cells in 100 μL HBSS intravenously via the tail vein. On day 4 post tumor cells injection, mice were injected intravenously via the retro orbital plexus with 2.5×10⁶ BCMA-targeted single domain CAR-T cells, un-transduced mock T cells from the same donor (normalized to the highest CAR-T dose), or with vehicle control. Tumor growth in mice was monitored through bioluminescent (IVIS imager; PerkinElmer). Mice were monitored daily for signs of illness and sacrificed immediately if they met pre-specified humane endpoints including but not limited to hind-limb paralysis, respiratory distress, or 30% body weight loss as approved by the Animal Care Committee of the Research Center.

In order to monitor tumor growth in mice, whole body luminescent images were taken on a regular basis using IVIS Lumina Ill (Perkin Elmer, USA). Mice were given an intraperitoneal injection of 150 mg/kg Redi-Ject D-Luciferin Bioluminescent substrate (Perkin Elmer) 5 min before anesthesia (3% isoflurane) and imaged at the peak of photon emission excitation 580 and emission 670. Images were used to calculate the relative amounts of luciferase gene expression by quantifying the total flux (photons/second) and analyzed Living Image Software (Caliper Life Sciences, MA).

### Results

To assess the activity of BCMA-binding single domain-CAR-T in a xenogeneic model, 8 week old NOD/SCID mice were inoculated intravenously with 50,000 Ramos-FLUC cells on day 0, and subsequently treated by retro-orbital injection with 2.5×10⁶ BCMA-targeted single domain-CAR-T cells (BCMA-E7, A6, H2, H4, or V8) generated from healthy human donor T cells as described above, or Mock-transduced T cells (no lentivirus) without CAR expression on day 4. Mice were imaged by bioluminescence in vivo imaging.

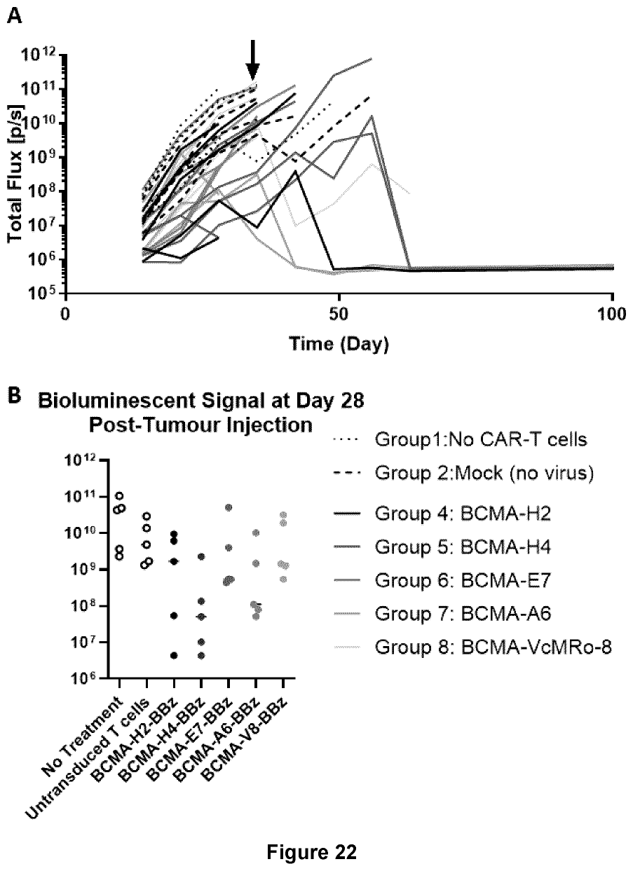

FIG. 22 depicts result of tumor burden in mice that were inoculated with Ramos-FLUC and treated with various CAR-T cells. Mice were monitored for tumor burden by quantifying bioluminescence using IVIS Lumina Ill. Graph depicts the total flux (photons/second) in individual animals within each treatment group (FIG. 22, panel A) over the course of the experiment; the bioluminescent reading from the final experimental time point where mice from all groups were alive is shown in FIG. 22, panel B. Mice treated with BCMA-H2, BCMA-H4, or BCMA-A6 CAR-T cells showed a moderate reduction in tumor burden compared to mice receiving mock T cells with few mice in groups treated with BCMA-H2, BCMA-H4 or BCMA-A6 H4 showing tumor resolution.

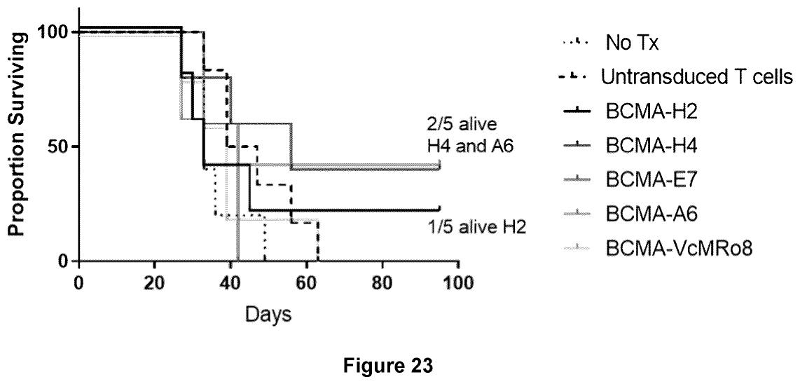

FIG. 23 depicts the proportion of surviving animals in each treatment group throughout the course of the experiment. Mice were monitored daily for signs of illness and sacrificed immediately if they met pre-specified humane endpoints as described above. At the end of the experimental monitoring period, 2 out of 5 mice were alive with no sign of disease in animals treated with BCMA-H4 or BCMA-A6 CAR-T cells, and 1 out of 5 mice were alive with no sign of disease in animals treated with BCMA-H2 CAR-T cells. Although not statistically significant, this data shows potential therapeutic benefit of these constructs.

### Discussion

NSG mice are widely used to study the interactions between the human immune system and cancer, a practical platform for evaluating immunotherapeutics in the context of human immune cells and human tumors. Overall, these results clearly demonstrate anti-cancer activity of BCMA-targeting single domain CAR modified T cells in vivo, similar to in vitro, and demonstrate therapeutic potential of these antibodies as tumor targeting moieties within CAR-T cells. Their ability to effectively and specifically target cells expressing BCMA antigen also provides evidence for their therapeutic potential beyond CAR-T therapy.

### Example 5: BiTE Constructs

### Introduction

Similar to chimeric antigen receptor technology, novel antigen binding elements can also be linked to CD3-engaging antibody elements in order generate a soluble molecule that can simultaneously bind T cells and cellular target molecules, resulting in an antigen-specific T cell activation signal. This type of molecule, referred to as a bi-specific T cell engagers, is exemplified by Blinatumomab, wherein a single molecule simultaneously engages human CD19 and human CD3; used as a therapy for CD19 expressing B-cell family malignancies. In order to assess whether the human BCMA-specific single domain antibodies generated herein could be used in such a bi-specific T cell engager molecule, molecules were generated wherein one end of the molecule was comprised of a BCMA-specific single domain antibody sequence and the other end was comprised of a CD3-engager molecule. These novel bi-specific T cell engagers were then screened for non-specific and antigen-specific induction of T cell activation and T cell killing of target cells.

### Materials and Methods

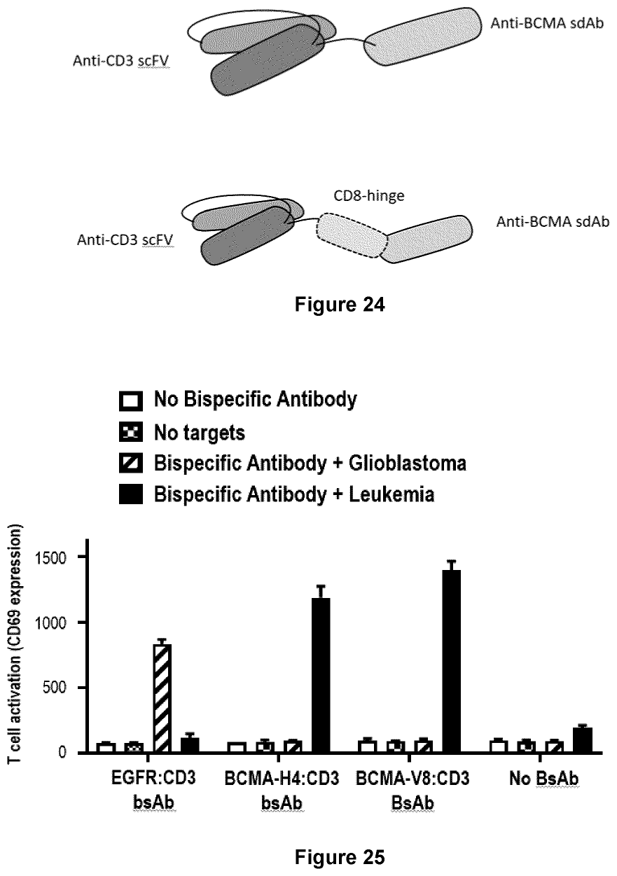

Single domain antibody antigen binding sequences were transferred to a modular bi-specific T cell engager DNA sequence (see SEQ ID NO: 76) within a plasmid backbone; the DNA sequence used contains restriction sites to allow efficient recombination wherein the antigen binding domain could be replaced with the novel BCMA-antigen binding domain (ABD) sequences. Specific bi-specific T cell engager design used was as follows: Human CD28 signal peptide (SEQ ID NO: 69), sdAb antibody (ABD) (e.g., any one of SEQ ID NOs: 40 to 58), flexible linker domain (SEQ ID NO: 70), human CD8 hinge domain (SEQ ID NO: 71), short flexible linker domain (SEQ ID NO: 75), and a CD3-specific single chain variable fragment sequence (see SEQ ID No: 78 for an sequence of an example BCMA-bispecific immune engager construct comprising sdAb H4). A model of BCMA-CD3 bi-specific T cell engager molecules with or without the inclusion of a hinge/spacer domain is provided (FIG. 24). Constructs were generated using golden gate assembly and confirmed using Sanger sequencing before proceeding to downstream testing.

To generate purified protein forms of bi-specific T cell engager molecules, plasmid DNA containing various constructs were transfected into HEK293T cells using polyethylenimine via standard process. Transfected cells were placed in cell culture and supernatant was collected over several days. Supernatant from BCMA-CD3 bispecific antibodies or a control EGFR-CD3 bi-specific antibody were then tested for bi-specific T cell engager activity by placing supernatant directly on Jurkat cells alone or in co-culture with BCMA-positive (Ramos) or BCMA-negative (U87vIII) target cells and incubated under standard conditions overnight. Jurkat cells were then examined for T cell activation using antibody staining for the human CD69 marker and flow cytometric analysis (FIG. 25). Results demonstrate that when delivered in solution, a BCMA-sdAb targeted bi-specific T cell engager can induce target dependent T cell activation, with varying activity between different constructs.

To test whether these results extend to induction of specific anti-tumour responses in primary human T cells, the novel bi-specific T cell engager containing supernatants generated above was next utilized in an assay with primary T cells. Specifically, T cells were isolated from human donor blood and polyclonally expanded for 10 days. Following polyclonal expansion, T cells were placed in co-culture with stable fluorescent protein (NucLight; Sartorius, USA) expressing BCMA-positive target cells (Raji or Ramos) in the presence of supernatant containing various bi-specific T cell engagers or control supernatant (Mock). Co-cultures were then monitored for target cell growth using IncuCyte (Sartorius, USA) live microscopy device. Using automated cell counting of fluorescently labelled target cells, the relative growth of target cells was quantified over 3 days (FIG. 24). Results demonstrate that BCMA-sdAb containing bi-specific T cell engager molecules can re-target cytolytic human T cell responses against BCMA-expressing target cells, and that activity is improved when a CD8-hinge/spacer domain is included in some constructs.

### Results

FIG. 24 depicts the molecular structure of BCMA-specific single domain antibody bi-specific T cell engager proteins with or without the inclusion of an additional hinge/spacer domain; with a BCMA-sdAb sequence at the 5′ end of a DNA construct, followed by a linker sequence which can be of varying composition, followed by a CD3-specific single chain variable fragment.

FIG. 25 depicts the results of Jurkat cell bi-specific T cell engager activation activity assay wherein HEK293T supernatants containing various bi-specific T cell engager molecules was placed on top of co-cultures containing Jurkat cells and BCMA-positive (Ramos) or BCMA-negative (U87vIII) target cells. Graphs depict the average CD69-specific antibody staining of Jurkat cells as measured by flow cytometry. Error bars present the standard error of the mean over 2 duplicate co-culture wells. Results demonstrate BCMA-antigen specific activation of T cells in the presence of novel BCMA-sdAb bi-specific T cell engager molecules.

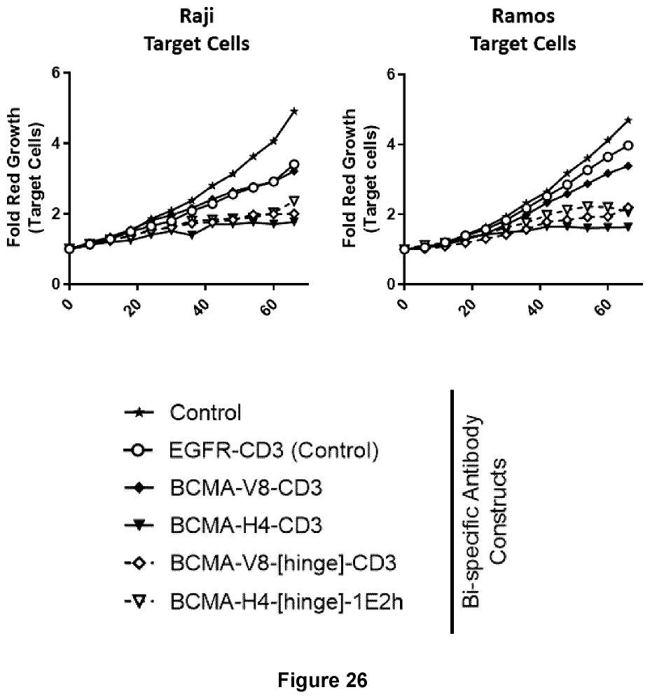

FIG. 26 depicts the results of a bi-specific T cell engager activity assay using primary human T cells in co-culture with BCMA-positive target cells (Ramos). As described above donor blood derived T cells were placed in co-culture with fluorescently labelled target cells in the presence of control (mock) or BCMA-specific bi-specific T cell engager containing supernatants and examined hourly over 3 days via live fluorescence microscopy. Graphs depict the fold growth of fluorescently labelled target cells as determined using automated cell counting. Error bars present the standard error of the mean over 2 duplicate co-culture wells. Results demonstrate T-cell mediated tumour growth suppression in the presence of BCMA-sdAb targeted bi-specific T cell engager molecules.

### Discussion

Overall these results exemplify that BCMA-specific single domain binders can generate strong antigen-driven T cell activation signaling when combined in a bi-specific T cell engager molecule. BCMA-sdAb targeted bi-specific T cell engager molecules are demonstrated to drive target specific T cell activation and direct target cell killing by primary human T cells. While exemplary data is provided for 2 BCMA-specific single domain antibodies, this data indicates that additional high affinity BCMA-binders described in this application are likely to have similar activity. These results can be extended to multivalent antibodies generally. Furthermore, molecular optimization may be performed in order to further increase functionality of bi-specific T cell engager molecules. In addition, combining multiple BCMA-specific single domain antibody sequences in a single molecule may be an effective strategy to increase target-specific activating activity.

### GENERAL DISCUSSION OF EXAMPLES

This is a demonstration of novel single domain antibodies for application in BCMA targeted immunotherapies with specific data driven evidence for their application in CAR-T and bi-specific T cell engager treatment modalities. Single domain antibodies offer significant advantage over the single-chain variable fragment antibodies which are typically used in the antigen recognition domain of CAR constructs, including significantly smaller size, higher homology with human antibody sequences, enhanced modularity, and ability to target epitopes which may not be accessible to scFvs. This invention may later be combined with other single domain antibodies targeting antigen that are co-expressed with BCMA to generate therapeutic construct targeting B-cell related disease indications; e.g. cancer, autoimmune diseases.

In the preceding description, for purposes of explanation, numerous details are set forth in order to provide a thorough understanding of the embodiments. However, it will be apparent to one skilled in the art that these specific details may not be required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

All references referred to herein are expressly incorporated by references in their respective entireties.
Bera T K. Anti-BCMA Immunotoxins: Design, Production, and Preclinical Evaluation. Biomolecules. 2020; 10(10):1387.
Baral T N, Arbabi-Ghahroudi M. Expression of single-domain antibodies in bacterial systems. Methods Mol Biol. 2012; 911:257-75.
Feldhaus et al., 2003, Nat. Biotechnol. Vol. 21, 163-170.
Bloemberg, Darin et al. A High-Throughput Method for Characterizing Novel Chimeric Antigen Receptors in Jurkat Cells. Molecular Therapy—Methods & Clinical Development. 2020, 16:238-254
Vallera et al. IL-15 Trispecific Killer Engagers (TriKEs) Make Natural Killer Cells Specific to CD33+ Targets While Also Inducing In Vivo Expansion, and Enhanced Function. Clinical Cancer Research. 2012; 22(14): 3440-50.
Gleason et al. Bispecific and Trispecific Killer Cell Engagers Directly Activate Human NK Cells Through CD16 Signaling and Induce Cytotoxicity and Cytokine Production. Mol Cancer Ther. 2012; 11(12): 2674-84.
Gauthier et al. Multifunctional Natural Killer Cell Engagers Targeting NKp46 Trigger Protective Tumor Immunity. Cell. 2019; 177(7): 1701-13.
Stoiber et al. Limitations in the Design of Chimeric Antigen Receptors for Cancer Therapy. Cells. 2012; 8(5): 472.
van der Stegen et al. The pharmacology of second-generation chimeric antigen receptors. Nat Rev Drug Discov. 2019; 14(7): 499-509.

TABLE 4
Table of Sequences

| Seq. | | | |
|---|---|---|---|
| # | Description | | Sequence |
| 1 | hBCMA-E7 | CDR1 | GRATDHFV |
| 2 | hBCMA-E7 | CDR2 | RVWSGGSP |
| 3 | hBCMA-E7 | CDR3 | AATKDIMSRSYDY |
| 4 | hBCMA-H2 | CDR1 | GRTSNNFV |
| 5 | hBCMA-H2 | CDR2 | RVWSGSTP |
| 6 | hBCMA-H2 | CDR3 | AATKDIMSRSYDY |
| 7 | hBCMA-V3 | CDR1 | GRASNHFV |
| 8 | hBCMA-V3 | CDR2 | RVWSGGSP |
| 9 | hBCMA-V3 | CDR3 | AATKDIMSRSYDY |
| 10 | hBCMA-B5 | CDR1 | GSIFGTYN |
| 11 | hBCMA-B5 | CDR2 | ISSAGNT |
| 12 | hBCMA-B5 | CDR3 | NGAPWADAEVKVYN |
| 13 | hBCMA-H4 | CDR1 | GDSFGAYA |
| 14 | hBCMA-H4 | CDR2 | ISSAGNT |
| 15 | hBCMA-H4 | CDR3 | NGAPWADEPVKVWN |
| 16 | hBCMA-H1 | CDR1 | GSGFGTHV |
| 17 | hBCMA-H1 | CDR2 | ISSAGST |
| 18 | hBCMA-H1 | CDR3 | NGAPWADEPVKVWN |
| 19 | hBCMA-F2 | CDR1 | GNSFGAYN |
| 20 | hBCMA-F2 | CDR2 | ISSTGNT |
| 21 | hBCMA-F2 | CDR3 | NGAPWGDDPVKVWS |
| 22 | hBCMA-A6 | CDR1 | GSIFDAYN |
| 23 | hBCMA-A6 | CDR2 | ISSAGTT |
| 24 | hBCMA-A6 | CDR3 | NGAPWGDAPVKVED |
| 25 | hBCMA | CDR1 | GDPITVAT |
| | VcMRo1(V1/V6) | | |
| 26 | hBCMA | CDR2 | ISSAGST |
| | VcMRo1(V1/V6) | | |
| 27 | hBCMA | CDR3 | NGAPWGDAPVKVWT |
| | VcMRo1(V1/V6) | | |
| 28 | hBCMA-D2 | CDR1 | GSIFGTYN |
| 29 | hBCMA-D2 | CDR2 | ISSAGNT |
| 30 | hBCMA-D2 | CDR3 | NGAPWGDALVKVWN |
| 31 | hBCMA-V8 | CDR1 | GTIFSRNA |
| 32 | hBCMA-V8 | CDR2 | ITIGGTT |
| 33 | hBCMA-V8 | CDR3 | NADPEGSWNWVRRGDY |
| 34 | hBCMA-2F10 | CDR1 | GHTTKNFV |
| 35 | hBCMA-2F10 | CDR2 | RVWSGGSP |
| 36 | hBCMA-2F10 | CDR3 | AATKDIMSRGYVY |
| 37 | hBCMA-3F2 | CDR1 | GHTSNQFV |
| 38 | hBCMA-3F2 | CDR2 | RFWGGGSP |
| 39 | hBCMA-3F2 | CDR3 | AATKDILSRGYDY |
| 40 | hBCMA-E7 | Full length | QVQLVESGGGLVQTGDSLRLACTISGRATDHFVMAWERRA |
| | | | PGKEREYVATRVWSGGSPYYLDSVKGRFAIAIDNAKNTAY |
| | | | LQMNNLKPEDTAVYYCAATKDIMSRSYDYWGLGTQVTVSS |
| 41 | hBCMA-H2 | Full length | QVKAEESGGGLVRPGDSLRLTCTISGRTSNNFVMAWFRRT |
| | | | PGKEREYVATRVWSGSTPYYHDSVKGRFTISIDDDKNTAY |
| | | | LQMNSLKPEDTAVYYCAATKDIMSRSYDYWGLGTQVTVSS |
| 42 | hBCMA-V3 | Full length | EVQLEQSGGGLVQTGDSLRLTCTIPGRASNHFVMAWFRRA |
| | (hBCMA- | | PGKEREFVATRVWSGGSPYYSDSVKGRFAIAIDNAKNTAY |
| | VcMRo3) | | LQMNSLKPEDTAVYYCAATKDIMSRSYDYWGLGTQVTVSS |
| 43 | hBCMA-B5 | Full length | QVKLEESGGGLVQPGGSLRLSCAASGSIFGTYNMGWYRQA |
| | | | PGKQRELVAAISSAGNTFYRDSVKGRFTVSRDNAKNTVYL |
| | | | QMDRLKYEDTAVYNCNGAPWADAEVKVYNWGQGTQVTVSS |
| 44 | hBCMA-H4 | Full length | QVQLVESGGGLVQPGGSLRLSCAASGDSFGAYAMGWYRQA |
| | | | PGKQRELVAAISSAGNTFYRDSVKGRFTVSRNNAKNAMYL |
| | | | QMDRLKPEDTAVYQCNGAPWADEPVKVWNWGLGTQVTVSS |
| 45 | hBCMA-H1 | Full length | QVQLVESGGGLVQPGGSLRLSCAASGSGFGTHVMGWYRQA |
| | | | PGKPRELVAAISSAGSTFYRDSVKGRFTVSRDNAKNTMYL |
| | | | QMDRLKPEDTAVYYCNGAPWADEPVKVWNWGQGTQVTVSS |
| 46 | hBCMA-F2 | Full length | QVKLEESGGGLVKPGGSLRLSCGASGNSFGAYNMGWYRQA |
| | | | PGKQRELVAAISSTGNTFYRDSVRGRFTVSRDNAKSTMSL |
| | | | QMERLKPEDTAVYLCNGAPWGDDPVKVWSWGQGTQVTVSS |
| 47 | hBCMA-A6 | Full length | QVQLVESGGGLVQPGGSLRLSCVASGSIFDAYNMGWYRQA |
| | | | PGKQRELVAAISSAGTTFYRDSVKGRFTVSRNNAKNTMYL |
| | | | QMDRLRPEDTAVYDCNGAPWGDAPVKVEDWGQGTQVTVSS |
| 48 | hBCMA | Full length | EVQLQQSGGGLVQAGESLTISCAVFGDPITVATMGWYRQA |
| | VcMRo1 | | PGKLRELVAAISSAGSTFYRDSVRGRFTVSRDNAKSTMYL |
| | (V1/V6) | | QMDRLKVEDTAVYSCNGAPWGDAPVKVWTWGEGTQVTVSS |
| 49 | hBCMA-D2 | Full length | QAQVQLVESGGGLVQPGGSLRLSCAASGSIFGTYNMGWYR |
| | | | QAPGKQRELVAAISSAGNTFYRDSVKGRFTVSRDNAKNTM |
| | | | YLQMDRLKYEDTAVYNCNGAPWGDALVKVWNWGQGTQVTV |
| | | | SS |
| 50 | hBCMA-V8 | Full length | QVQLVESGGGLVQPGGSLRLSCAASGTIFSRNALAWFRQA |
| | (VcMR08 | | PGKQRELVAHITIGGTTVYKDSVKGRFTISRDNAKNTVYL |
| | (VF7/VF8)) | | QMDALKPEDTAVYYCNADPEGSWNWVRRGDYWGQGTQVTV |
| | | | S |
| 51 | hBCMA-2F10 | Full length | QVQLVESGGGLVQSGNSLRLSCSISGHTTKNFVMAWFRRA |
| | | | PGKERAIVATRVWSGGSPWYSESAKGRFTISIDDARNTAY |
| | | | LQMNNLKPEDTAVYYCAATKDIMSRGYVYWGLGTQVTVSS |
| 52 | hBCMA-3F2 | Full length | QVQLVESGGRLVQTGDSLRLTCEISGHTSNQFVLAWERRP |
| | | | PGKEREVVATRFWGGGSPYYSDSVRGRFAIAIDDAKNTAY |
| | | | LQMSSLKPEDTAVYYCAATKDILSRGYDYWGQGTQVTVSS |
| 53 | hBCMA-A6 | Full length | QVQLVESGGGLVQPGGSLRLSCVASGSIFDAYNMGWYRQA |
| | used in CAR | | PGKQRELVAAISSAGTTFYRDSVKGRFTVSRNNAKNTMYL |
| | | | QMDRLRPEDTAVYDCNGAPWGDAPVKVEDWGQGTQVTVSS |
| 54 | hBCMA-E7 | Full length | QVQLVESGGGLVRPGDSLRLTCTISGRTSNNFVMAWFRRT |
| | used in CAR | | PGKEREYVATRVWSGSTPYYHDSVKGRFTISIDDDKNTAY |
| | | | LQMNSLKPEDTAVYYCAATKDIMSRSYDYWGQGTQVTVSS |
| 55 | BCMA-H2 used | Full length | QVQLVESGGGLVRPGDSLRLTCTISGRTSNNFVMAWERRT |
| | in CAR | | PGKEREYVATRVWSGSTPYYHDSVKGRFTISIDDDKNTAY |
| | | | LQMNSLKPEDTAVYYCAATKDIMSRSYDYWGQGTQVTVSS |
| 56 | BCMA-H4 used | Full length | QVQLVESGGGLVQPGGSLRLSCAASGDSFGAYAMGWYRQA |
| | in CAR | | PGKQRELVAAISSAGNTFYRDSVKGRFTVSRNNAKNAMYL |
| | | | QMDRLKPEDTAVYQCNGAPWADEPVKVWNWGLGTQVTVSS |
| 57 | BCMA-V3 used | Full length | QVQLVESGGGLVQTGDSLRLTCTIPGRASNHFVMAWERRA |
| | in CAR | | PGKEREFVATRVWSGGSPYYSDSVKGRFAIAIDNAKNTAY |
| | | | LQMNSLKPEDTAVYYCAATKDIMSRSYDYWGLGTQVTVSS |
| 58 | BCMA-V8 used | Full length | QVQLVESGGGLVQPGGSLRLSCAASGTIFSRNALAWFRQA |
| | in CAR | | PGKQRELVAHITIGGTTVYKDSVKGRFTISRDNAKNTVYL |
| | | | QMDALKPEDTAVYYCNADPEGSWNWVRRGDYWGQGTQVTV |
| | | | SS |
| 59 | Group 1 CDR1 Consensus | | GX₁X₂X₃X₄X₅FV, wherein: |
| | | | X₁ is R or H, |
| | | | X₂ is A or T, |
| | | | X₃ is T or S, |
| | | | X₄ is D, N, or K, and |
| | | | X₅ is H, N, or Q |
| 60 | Group 1 CDR2 Consensus | | RX₆WX₇GX₈X₉P, wherein: |
| | | | X₆ is V or F, |
| | | | X₇ is S or G, |
| | | | X₈ is G or S, and |
| | | | X₉ is S or T, |
| 61 | Group 1 CDR3 Consensus | | AATKDIX₁₀SRX₁₁YX₁₂Y, wherein: |
| | | | X₁₀ is M or L, |
| | | | X₁₁ is S or G, and |
| | | | X₁₂ is D or V; |
| 62 | Group 2 CDR1 Consensus | | GX₁X₂FGX₃X₄X₅, wherein: |
| | | | X₁ is S or D, |
| | | | X₂ is I, S, or G, |
| | | | X₃ is T or A, |
| | | | X₄ is Y or H, and |
| | | | X₅ is N, A, or V |
| 63 | Group 2 CDR2 Consensus | | ISSAGX₆T, wherein: |
| | | | X₆ is N or S |
| 64 | Group 2 CDR3 Consensus | | NGAPWADX₇X₈VKVX₉N, wherein: |
| | | | X₇ is A or E, |
| | | | X₉ is E or P, and |
| | | | X₉ is Y or W |
| 65 | Group 3 CDR1 Consensus | | GX₁X₂X₃X₄X₅X₆X₇, wherein: |
| | | | X₁ is N, S, or D, |
| | | | X₂ is S, I, or P, |
| | | | X₃ is F or I, |
| | | | X₄ is G, D, or T, |
| | | | X₅ is A, V, or T, |
| | | | X₆ is Y or A, and |
| | | | X₇ is N or T |
| 66 | Group 3 CDR2 Consensus | | ISSX₈GX₉T, wherein: |
| | | | X₈ is T or A, and |
| | | | X₉ is N, T, or S |
| 67 | Group 3 CDR3 Consensus | | NGAPWGDX₁₀X₁₁VKVX₁₂X₁₃, wherein: |
| | | | X₁₀ is D or A, |
| | | | X₁₁ is P or L, |
| | | | X₁₂ is W or E, and |
| | | | X₁₃ is S, D, T, or N |
| 68 | CAR modular construct | | atgctcaggctgctcttggctctcaacttattcccttcaa |
| | | | ttcaagtaacaggaggGTCTTC |
| | | | . . . [ABD_sequence] . . . |
| | | | GAAGACttCCTTTGcGAGACGacGGTGGCGGGGGATCAGG |
| | | | TGGTGGAGGTAGCGGGGGAGGGGGCTCAGGCGGTACAACT |
| | | | ACGCCTGCACCTCGCCCACCGACCCCAGCACCAACCATCG |
| | | | CTTCACAGCCTTTGAGCCTGCGACCAGAGGCATGTCGCCC |
| | | | TGCTGCGGGCGGTGCCGTTCATACTCGCGGACTTGATTTT |
| | | | GCGTGTGACgtCGTCTCgccttctaagcccttttgggtgc |
| | | | tggtggtggttggtggagtcctggcttgctatagcttgct |
| | | | agtaacagtggcctttattattttctgggtgaggaaacgg |
| | | | ggcagaaagaaactcctgtatatattcaaacaaccattta |
| | | | tgCgaccagtacaaactactcaagaggaagatggctgtag |
| | | | ctgccgatttccagaagaagaagaaggaggatgtgaactg |
| | | | ctgagagtgaagttcagcaggagcgcagacgcccccgcgt |
| | | | accagcagggccagaaccagctctataacgagctcaatct |
| | | | aggacgaagagaggagtacgatgttttggacaagCgacgt |
| | | | ggccgggaccctgagatggggggaaagccgcagagaagga |
| | | | agaaccctcaggaaggcctgtacaatgaactgcagaaaga |
| | | | taagatggcggaggcctacagtgagattgggatgaaaggc |
| | | | gagcgccggaggggcaaggggcacgatggcctttaccagg |
| | | | gActcagtacagccaccaaggacacctacgacgcccttca |
| | | | catgcaggccctgccccctcgcGCTAGCGCCACGAACTTC |
| | | | TCTCTGTTAAAGCAAGCAGGCGACGTGGAAGAAAACCCCG |
| | | | GTCCCATGGTGAGCAAGGGCGAGGAGGACAACATGGCCAG |
| | | | CCTGCCCGCCACCCACGAGCTGCACATCTTCGGCAGCATC |
| | | | AACGGCGTGGACTTCGACATGGTGGGCCAGGGCACCGGCA |
| | | | ACCCCAACGACGGCTACGAGGAGCTGAACCTGAAGAGCAC |
| | | | CAAGGGCGACCTGCAGTTCAGCCCCTGGATtCTGGTGCCC |
| | | | CACATCGGCTACGGCTTCCACCAGTACCTGCCCTACCCCG |
| | | | ACGGCATGAGCCCCTTCCAGGCCGCCATGGTGGACGGCAG |
| | | | CGGCTACCAGGTGCACAGGACCATGCAGTTCGAGGACGGC |
| | | | GCCAGCCTGACCGTGAACTACAGGTACACCTACGAGGGCA |
| | | | GCCACATCAAGGGCGAGGCCCAGGTGAAGGGCACCGGCTT |
| | | | CCCCGCCGACGGCCCCGTGATGACCAACAGCCTGACCGCC |
| | | | GCCGACTGGTGCAGGAGCAAAAAGACCTACCCCAACGACA |
| | | | AGACCATCATCAGCACCTTCAAGTGGAGCTACACCACCGG |
| | | | CAACGGCAAGAGGTACAGGAGCACCGCCAGGACCACCTAC |
| | | | ACCTTCGCCAAGCCCATGGCCGCCAACTACCTGAAGAACC |
| | | | AGCCCATGTACGTGTTCAGAAAGACCGAGCTGAAGCACAG |
| | | | CAAGACCGAGCTGAACTTCAAGGAGTGGCAGAAGGCCTTC |
| | | | ACCGACGTGATGGGCATGGACGAGCTGTACAAGCCCAAGA |
| | | | AGAAGAGGAAGGTGGAGGACCCCCCCGCCGCCAAGAGGGT |
| | | | GAAGCTGGACTaa |
| 69 | Human CD28 Signal Peptide | | MLRLLLALNLFPSIQVTG |
| 70 | Synthetic Flexible Linker | | GGGGSGGGGSGGGGSGG |
| | Domain (exemplary) | | |
| 71 | Human CD8 Hinge Domain | | TTTPAPRPPTPAPTIASQPLSLRPEACRPAAGGAVHTRGL |
| | (exemplary) | | DFACD |
| 72 | Human CD28 Transmembrane | | PSKPFWVLVVVGGVLACYSLLVTVAFIIFWVR |
| | Domain (exemplary) | | |
| 73 | Human 4-1BB Costimulatory | | KRGRKKLLYIFKQPFMRPVQTTQEEDGCSCRFPEEEEGGC |
| | Domain (exemplary) | | EL |
| 74 | Human CD3zeta Signaling | | LRVKFSRSADAPAYQQGONQLYNELNLGRREEYDVLDKRR |
| | Domain (exemplary) | | GRDPEMGGKPQRRKNPQEGLYNELQKDKMAEAYSEIGMKG |
| | | | ERRRGKGHDGLYQGLSTATKDTYDALHMQALPPR |
| 75 | Short flexible linker amino acid | | GGGGS |
| | sequence (exemplary) | | |
| 76 | Bispecific T cell engager | | ATGGAGTTTGGGCTGAGCTGGGTTTTCCTCGTTGCTCTTT |
| | modular construct DNA | | TTAGAGGTGTCCAGTGTACAGGAGGGTCTTCG . . . |
| | sequence | | [ABD_sequence] . . . |
| | | | gaagacttccttggaggaggcggaagtCAAGT |
| | | | GCAGTTGCAACAACCAGGTGCCGAGCTTGGGAAACCGGGT |
| | | | ACCAGCGTTAAACTCTCTTGCAAAGCATCCGGTTATACCT |
| | | | TTACTTCTTACTGGATCCATTGGGTCAAACAACGCCCAGG |
| | | | CCAGGGTCTCGAATGGGTGGGCAACATCAACCCAAACAGC |
| | | | GGCTCTATCAATTATAACGAAAAGTTTAAGAATAAAGCAA |
| | | | CTCTCACGGTGGATAAGAGCAGCTCAACCGCTTATATGCA |
| | | | ATTGAGTACTTTGACCAGTGAAGATTTCGCTGTGTATTAT |
| | | | TGCACGCGCGATACATCCGGACAGTATTATTTCGATTATT |
| | | | GGGGCCAGGGGACAACCCTGACAGTGTCTAGCggcggtgg |
| | | | tgggtcaggcggcggtgggagcggaggaggtggaagcGAT |
| | | | ATCGTAATGAGTCAATCCCCTAGCAGCTTGGCGGTAAGTG |
| | | | CTGGGGAACGCGTTACAATGTCCTGTAAGAGCTCTCAATC |
| | | | TTTGCTGAATTCCAGGACTAGGAAGAATTATCTGGCCTGG |
| | | | TATCAACAAAAGCCCGGACAAAGTCCAAAGCTCCTGATTT |
| | | | ATTGGGCTAGCACCAGAGAGTCAGGAGTACCAGACCGGTT |
| | | | TACTGGAAGCGGGTCAGGAACCGACTTTACCCTGACAATA |
| | | | TCCAGCGTCCAAGCAGAAGATCTTGCCGTGTACTATTGTA |
| | | | TTCAGTCCTACCACTTGCGCACTTTTGGGGGGGGAACAAA |
| | | | ACTCGAGATAAAGCACCACCACCACCACCACTAG |
| 77 | BCMA-A6-H4-BBz CAR amino | | MLRLLLALNLFPSIQVTGQVQLVESGGGLVRPGDSLRLTC |
| | acid sequence | | TISGRTSNNFVMAWFRRTPGKEREYVATRVWSGSTPYYHD |
| | Exemplary sequence for a multi- | | SVKGRFTISIDDDKNTAYLQMNSLKPEDTAVYYCAATKDI |
| | binder CAR construct | | MSRSYDYWGQGTQVTVSSPSGGGGQVQLVESGGGLVQPGG |
| | sdAb sequence in bold | | SLRLSCAASGDSFGAYAMGWYRQAPGKQRELVAAISSAGN |
| | | | TFYRDSVKGRFTVSRNNAKNAMYLQMDRLKPEDTAVYQCN |
| | | | GAPWADEPVKVWNWGLGTQVTVSSPSTTTPAPRPPTPAPT |
| | | | IASQPLSLRPEACRPAAGGAVHTRGLDFACDPSKPFWVLV |
| | | | VVGGVLACYSLLVTVAFIIFWVRKRGRKKLLYIFKOPFMR |
| | | | PVQTTQEEDGCSCRFPEEEEGGCELLRVKFSRSADAPAYQ |
| | | | QGQNQLYNELNLGRREEYDVLDKRRGRDPEMGGKPQRRKN |
| | | | PQEGLYNELQKDKMAEAYSEIGMKGERRRGKGHDGLYQGL |
| | | | STATKDTYDALHMQALPPR |
| 78 | BCMA-H4-Linker-CD8h-Short | | MEFGLSWVFLVALFRGVQCTGQVQLVESGGGLVQPGGSLR |
| | linker-GG-CD3scFv | | LSCAASGDSFGAYAMGWYRQAPGKQRELVAAISSAGNTFY |
| | Exemplary sequence for a | | RDSVKGRFTVSRNNAKNAMYLQMDRLKPEDTAVYQCNGAP |
| | BCMA-bispecific immune | | WADEPVKVWNWGLGTQVTVSSGGGGSGGGGSGGGGSGGTT |
| | engager construct | | TPAPRPPTPAPTIASQPLSLRPEACRPAAGGAVHTRGLDF |
| | sdAb in bold | | ACD . . . [CD3-specific_scFv_ |
| | sdAb sequence in bold | | Sequence] . . . HHHHHH |
| 79 | hBCMA-2C3 | Full length | QVKLEESGGGLVQTGDSLRLACTISGRATDHFVMAWERRA |
| | | | PGKEREYVATRVWSGGSPYYLDSAKGRFAIAIDNAKNTAY |
| | | | LQMNNLKPEDTAVYYCAATKDIMSRSYDYWGLGTQVTVSS |
| 80 | hBCMA-4D1 | Full length | QVQLVESGGGLVRPGDSLRLTCTISGRTSNNFVMAWERRT |
| | | | PGKEREYVATRVWSGSTPYYHDSVKGRFTISIDDDKNTAY |
| | | | LQMNSLKPEDTAVYYCAATKDIMSRSYDYWGLGTQVTVSS |

| | | |
|---|---|---|
| 81 | VH sense primer | CATGTGTAGACTCGCGGCCCAGCCGGCCATGGCC |
| 82 | VH antisense primer | CATGTGTAGATTCCTGGCCGGCCTGGCCTGAGGAGACGGT |
| | | GACCTGG |

1. An isolated single domain antibody (sdAb), which binds specifically to human B-cell maturation antigen (BCMA), the sdAb comprising:
a) a CDR1 amino acid sequence GX₁X₂X₃X₄YX₅FV (SEQ ID NO: 59), wherein:
X₁ is R or H,
X₂ is A or T,
X₃ is T or S,
X₄ is D, N, or K, and
X₅ is H, N, or Q,
a CDR2 amino acid sequence RX₆WX₇GX₈X₉P (SEQ ID NO: 60), wherein:
X₆ is V or F,
X₇ is S or G,
X₈ is G or S, and
X₉ is S or T, and
a CDR3 amino acid sequence AATKDIX₁₀SRX₁₁YX₁₂Y (SEQ ID NO: 61), wherein:
X₁₀ is M or L,
X₁₁ is S or G, and
X₁₂ is D or V;
b) a CDR1 amino acid sequence GX₁X₂FGX₃X₄X₅ (SEQ ID NO: 62), wherein:
X₁ is S or D,
X₂ is I, S, or G,
X₃ is T or A,
X₄ is Y or H, and
X₅ is N, A, or V,
a CDR2 amino acid sequence ISSAGX₆T (SEQ ID NO: 63), wherein:
X₆ is N or S, and
a CDR3 amino acid sequence NGAPWADX₇X₈VKVX₉N (SEQ ID NO: 64), wherein:
X₇ is A or E,
X₈ is E or P, and
X₉ is Y or W;
c) a CDR1 amino acid sequence GX₁X₂X₃X₄X₅X₆X₇ (SEQ ID NO: 65), wherein:
X₁ is N, S, or D,
X₂ is S, I, or P,
X₃ is F or I,
X₄ is G, D, or T,
X₅ is A, V, or T,
X₆ is Y or A, and
X₇ is N or T,
a CDR2 amino acid sequence ISSX₈GX₉T (SEQ ID NO: 66), wherein:
X₈ is T or A, and
X₉ is N, T, or S, and
a CDR3 amino acid sequence NGAPWGDX₁₀X₁₁VKVX₁₂X₁₃ SEQ ID NO: 67), wherein:
X₁₀ is D or A,
X₁₁ is P or L,
X₁₂ is W or E, and
X₁₃ is S, D, T, or N;
d) a CDR1 amino acid sequence as set forth in SEQ ID NO: 31,
a CDR2 amino acid sequence as set forth in SEQ ID NO: 32, and
a CDR3 amino acid sequence as set forth in SEQ ID NO: 33 (from hBCMA VcMRo8 (VF7/VF8));
or
e) a CDR1 amino acid sequence as set forth in SEQ ID NO: 34,
a CDR2 amino acid sequence as set forth in SEQ ID NO: 35, and
a CDR3 amino acid sequence as set forth in SEQ ID NO: 36 (from hBCMA-A3).

2. An isolated single domain antibody (sdAb), which binds specifically to human B-cell maturation antigen (BCMA), the sdAb comprising:
A)
i) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7),
ii) a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2),
iii) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3),
iv) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5),
v) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4),
vi) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1),
vii) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2),
viii) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6),
ix) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)),
x) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2),
xi) a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)),
xii) a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10), or
xiii) a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2).

3. An isolated single domain antibody (sdAb), which binds specifically to human B-cell maturation antigen (BCMA), the sdAb comprising:
A)
i) a CDR1 amino acid sequence as set forth in SEQ ID NO: 1, a CDR2 amino acid sequence as set forth in SEQ ID NO: 2, and a CDR3 amino acid sequence as set forth in SEQ ID NO: 3 (from hBCMA-E7),
ii) a CDR1 amino acid sequence as set forth in SEQ ID NO: 4, a CDR2 amino acid sequence as set forth in SEQ ID NO: 5, and a CDR3 amino acid sequence as set forth in SEQ ID NO: 6 (from hBCMA-H2),
iii) a CDR1 amino acid sequence as set forth in SEQ ID NO: 7, a CDR2 amino acid sequence as set forth in SEQ ID NO: 8, and a CDR3 amino acid sequence as set forth in SEQ ID NO: 9 (from hBCMA-V3),
iv) a CDR1 amino acid sequence as set forth in SEQ ID NO: 10, a CDR2 amino acid sequence as set forth in SEQ ID NO: 11, and a CDR3 amino acid sequence as set forth in SEQ ID NO: 12 (from hBCMA-B5),
v) a CDR1 amino acid sequence as set forth in SEQ ID NO: 13, a CDR2 amino acid sequence as set forth in SEQ ID NO: 14, and a CDR3 amino acid sequence as set forth in SEQ ID NO: 15 (from hBCMA-H4),
vi) a CDR1 amino acid sequence as set forth in SEQ ID NO: 16, a CDR2 amino acid sequence as set forth in SEQ ID NO: 17, and a CDR3 amino acid sequence as set forth in SEQ ID NO: 18 (from hBCMA-H1),
vii) a CDR1 amino acid sequence as set forth in SEQ ID NO: 19, a CDR2 amino acid sequence as set forth in SEQ ID NO: 20, and a CDR3 amino acid sequence as set forth in SEQ ID NO: 21 (from hBCMA-F2),
viii) a CDR1 amino acid sequence as set forth in SEQ ID NO: 22, a CDR2 amino acid sequence as set forth in SEQ ID NO: 23, and a CDR3 amino acid sequence as set forth in SEQ ID NO: 24 (from hBCMA-A6),
ix) a CDR1 amino acid sequence as set forth in SEQ ID NO: 25, a CDR2 amino acid sequence as set forth in SEQ ID NO: 26, and a CDR3 amino acid sequence as set forth in SEQ ID NO: 27 (from hBCMA VcMRo1(V1/V6)),
x) a CDR1 amino acid sequence as set forth in SEQ ID NO: 28, a CDR2 amino acid sequence as set forth in SEQ ID NO: 29, and a CDR3 amino acid sequence as set forth in SEQ ID NO: 30 (from hBCMA-D2),
xi) a CDR1 amino acid sequence as set forth in SEQ ID NO: 31, a CDR2 amino acid sequence as set forth in SEQ ID NO: 32, and a CDR3 amino acid sequence as set forth in SEQ ID NO: 33 (from hBCMA VcMRo8 (VF7/VF8)),
xii) a CDR1 amino acid sequence as set forth in SEQ ID NO: 34, a CDR2 amino acid sequence as set forth in SEQ ID NO: 35, and a CDR3 amino acid sequence as set forth in SEQ ID NO: 36 (from hBCMA-2F10), or
xiii) a CDR1 amino acid sequence as set forth in SEQ ID NO: 37, a CDR2 amino acid sequence as set forth in SEQ ID NO: 38, and a CDR3 amino acid sequence as set forth in SEQ ID NO: 39 (from hBCMA-3F2); or
B)
CDR1, CDR2, and CDR3 amino acid sequences that are at least 80% identical to the
CDR1, CDR2, and CDR3 sequences defined in any one of part A) i) to xxviii).

4. An isolated single domain antibody (sdAb), which binds specifically to human B-cell maturation antigen (BCMA), the sdAb comprising:
a CDR3 amino acid sequence as set forth in SEQ ID NO: 3,
i) a CDR3 amino acid sequence as set forth in SEQ ID NO: 6,
ii) a CDR3 amino acid sequence as set forth in SEQ ID NO: 9,
iii) a CDR3 amino acid sequence as set forth in SEQ ID NO: 12,
iv) a CDR3 amino acid sequence as set forth in SEQ ID NO: 15,
v) a CDR3 amino acid sequence as set forth in SEQ ID NO: 18,
vi) a CDR3 amino acid sequence as set forth in SEQ ID NO: 21,
vii) a CDR3 amino acid sequence as set forth in SEQ ID NO: 24,
viii) a CDR3 amino acid sequence as set forth in SEQ ID NO: 27,
ix) a CDR3 amino acid sequence as set forth in SEQ ID NO: 30,
x) a CDR3 amino acid sequence as set forth in SEQ ID NO: 33,
xi) a CDR3 amino acid sequence as set forth in SEQ ID NO: 36, or
xii) a CDR3 amino acid sequence as set forth in SEQ ID NO: 39.

5. The isolated sdAb of claim 4, comprising:
a CDR1 amino acid sequence as set forth in SEQ ID NO: 1, and a CDR3 amino acid sequence as set forth in SEQ ID NO: 3,
i) a CDR1 amino acid sequence as set forth in SEQ ID NO: 4, and a CDR3 amino acid sequence as set forth in SEQ ID NO: 6,
ii) a CDR1 amino acid sequence as set forth in SEQ ID NO: 7, and a CDR3 amino acid sequence as set forth in SEQ ID NO: 9,
iii) a CDR1 amino acid sequence as set forth in SEQ ID NO: 10, and a CDR3 amino acid sequence as set forth in SEQ ID NO: 12,
iv) a CDR1 amino acid sequence as set forth in SEQ ID NO: 13, and a CDR3 amino acid sequence as set forth in SEQ ID NO: 15,
v) a CDR1 amino acid sequence as set forth in SEQ ID NO: 16, and a CDR3 amino acid sequence as set forth in SEQ ID NO: 18,
vi) a CDR1 amino acid sequence as set forth in SEQ ID NO: 19, and a CDR3 amino acid sequence as set forth in SEQ ID NO: 21,
vii) a CDR1 amino acid sequence as set forth in SEQ ID NO: 22, and a CDR3 amino acid sequence as set forth in SEQ ID NO: 24,
viii) a CDR1 amino acid sequence as set forth in SEQ ID NO: 25, and a CDR3 amino acid sequence as set forth in SEQ ID NO: 27,
ix) a CDR1 amino acid sequence as set forth in SEQ ID NO: 28, and a CDR3 amino acid sequence as set forth in SEQ ID NO: 30,
x) a CDR1 amino acid sequence as set forth in SEQ ID NO: 31, and a CDR3 amino acid sequence as set forth in SEQ ID NO: 33,
xi) a CDR1 amino acid sequence as set forth in SEQ ID NO: 34, and a CDR3 amino acid sequence as set forth in SEQ ID NO: 36, or
xii) a CDR1 amino acid sequence as set forth in SEQ ID NO: 37, and a CDR3 amino acid sequence as set forth in SEQ ID NO: 39.

6. The isolated sdAb of claim 2 or 3, comprising A) the amino acid sequence of any one of SEQ ID NOs: 40 to 58, 79, and 80 or B) an amino acid sequence that is at least 80% identical to any one of SEQ ID NOs: 40 to 58 across the full length thereof.

7. The isolated sdAb of claim 1 or 2, comprising A) the amino acid sequence of any one of SEQ ID NO: 40 to 58.

8. The isolated sdAb of any one of claims 1 to 7, which is a camelid sdAb, preferably a lama sdAb.

9. The isolated sdAb of any one of claims 1 to 5, which is humanized.

10. The isolated sdAb of any one of claims 1 to 5, which binds to an epitope in a portion of BCMA from Gly6 to Pro23, and preferably comprises:
the CDR1 amino acid sequence as set forth in SEQ ID NO: 1, the CDR2 amino acid sequence as set forth in SEQ ID NO: 2, and the CDR3 amino acid sequence as set forth in SEQ ID NO: 3 (from hBCMA-E7 or hBCMA-2C3),
the CDR1 amino acid sequence as set forth in SEQ ID NO: 4, the CDR2 amino acid sequence as set forth in SEQ ID NO: 5, and the CDR3 amino acid sequence as set forth in SEQ ID NO: 6 (from hBCMA-H2 or hBCMA-4D1), or
the CDR1 amino acid sequence as set forth in SEQ ID NO: 7, the CDR2 amino acid sequence as set forth in SEQ ID NO: 8, and the CDR3 amino acid sequence as set forth in SEQ ID NO: 9 (from hBCMA-V3).

11. The isolated sdAb of any one of claims 1 to 5, which binds to an epitope in a portion of BCMA from Gly6 to Tyr40, and preferably comprises:
the CDR1 amino acid sequence as set forth in SEQ ID NO: 22, the CDR2 amino acid sequence as set forth in SEQ ID NO: 23, and the CDR3 amino acid sequence as set forth in SEQ ID NO: 24 (from hBCMA-A6),
the CDR1 amino acid sequence as set forth in SEQ ID NO: 13, the CDR2 amino acid sequence as set forth in SEQ ID NO: 14, and the CDR3 amino acid sequence as set forth in SEQ ID NO: 15 (from hBCMA-H4), or
the CDR1 amino acid sequence as set forth in SEQ ID NO: 31, the CDR2 amino acid sequence as set forth in SEQ ID NO: 32, and the CDR3 amino acid sequence as set forth in SEQ ID NO: 33 (from hBCMA VcMRo8 (VF7/VF8)).

12. The isolated sdAb of any one of claims 1 to 11, which has an affinity for human BCMA of 2.5×10⁻⁷ nM or less, more preferably 3×10⁻⁸ nM or less, more preferably 9.6×10⁻⁹ nM or less, more preferably 9.3×10⁻¹⁰ nM or less, more preferably 7×10⁻¹² nM or less.

13. A single domain antibody (sdAb) that competes for specific binding to human BCMA with the isolated sdAb as defined in claim 3 or 7.

14. The sdAb of claim 13, which is humanized.

15. A nucleic acid molecule encoding the isolated single domain antibody as defined in any one of claims 1 to 14.

16. A recombinant polypeptide comprising one or more sdAb as defined in any one of claims 1 to 14.

17. A nucleic acid molecule encoding the recombinant polypeptide as defined in claim 16.

18. A multivalent antibody comprising:
a first antigen-binding portion comprising the sdAb as defined in any one of claims 1 to 14, and
a second antigen-binding portion.

19. The multivalent antibody of claim 18, wherein the second antigen-binding moiety binds specifically to a cell-surface marker of an immune cell.

20. The multivalent antibody of claim 19, wherein the cell-surface marker of the immune cell comprises a T-cell marker.

21. The multivalent antibody of claim 20, wherein the T-cell marker comprises human CD3.

22. The multivalent antibody of any one of claim 18 to 21, comprising in N-terminal to C-terminal direction:
the first antigen-binding portion,
an amino acid linker, and
the second antigen-binding portion.

23. The multivalent antibody of claim 22, which further comprises an N-terminal signal peptide.

24. The multivalent antibody of claim 23, wherein the signal peptide is a signal peptide from human CD28, preferably comprising SEQ ID NO: 69.

25. The multivalent antibody of any one of claims 22 to 24, wherein the amino acid linker comprises a polypeptide hinge from human CD8, preferably comprising SEQ ID NO: 71.

26. The multivalent antibody of any one of claims 22 to 25, which is encoded by SEQ ID NO: 76.

27. The multivalent antibody of claim 19, wherein the cell-surface marker of the immune cell marker comprises a natural killer (NK) cell marker.

28. The multivalent antibody of claim 27, wherein the NK cell marker comprises human CD16.

29. The multivalent antibody of claim 27 or 28, further comprising a cytokine for stimulating activation, expansion, and/or survival of NK cells.

30. The multivalent antibody of claim 29, wherein the cytokine for stimulating expansion of NK cells is interleukin-15 (IL15).

31. The multivalent antibody of any one of claims 27 to 30, further comprising a third antigen-binding portion that binds to a second NK cell marker.

32. The multivalent antibody of claim 31, wherein the second NK cell marker is human NKp46.

33. The multivalent antibody of any one of claims 27 to 30, further comprising at least a third antigen-binding portion that binds to a tumour-associated antigen, preferably wherein the tumour-associated antigen is distinct from human BCMA.

34. The multivalent antibody of any one of claims 22 to 26, further comprising at least a third antigen-binding portion that binds to a tumour-associated antigen, preferably wherein the tumour-associated antigen is distinct from human BCMA.

35. The multivalent antibody of claim 33 or 34, wherein the third antigen-binding portion comprises a V_{H}H, V_{NAR}, or an scVF.

36. The multivalent antibody of any one of claims 22 to 34, wherein the second antigen-binding portion comprises a V_{H}H, V_{NAR}, or an scVF.

37. The multivalent antibody of any one of claims 22 to 25, which comprises an antigen-binding portion that binds to human serum albumin.

38. The multivalent antibody of any one of claims 18 to 26, which is a bispecific T-cell engager.

39. The multivalent antibody of any one of claims 18, 27, and 28, which is a bispecific killer cell engager (BiKE).

40. The multivalent antibody of any one of claims 17, and 29 to 35, which is a trispecific killer cell engager (TriKE).

41. The multivalent antibody of any one of claims 18 to 40, wherein the sdAb comprises:
the CDR1 amino acid sequence as set forth in SEQ ID NO: 1, the CDR2 amino acid sequence as set forth in SEQ ID NO: 2, and the CDR3 amino acid sequence as set forth in SEQ ID NO: 3 (from hBCMA-E7 or hBCMA-2C3),
the CDR1 amino acid sequence as set forth in SEQ ID NO: 4, the CDR2 amino acid sequence as set forth in SEQ ID NO: 5, and the CDR3 amino acid sequence as set forth in SEQ ID NO: 6 (from hBCMA-H2 or hBCMA-4D1),
the CDR1 amino acid sequence as set forth in SEQ ID NO: 7, the CDR2 amino acid sequence as set forth in SEQ ID NO: 8, and the CDR3 amino acid sequence as set forth in SEQ ID NO: 9 (from hBCMA-V3),
the CDR1 amino acid sequence as set forth in SEQ ID NO: 22, the CDR2 amino acid sequence as set forth in SEQ ID NO: 23, and the CDR3 amino acid sequence as set forth in SEQ ID NO: 24 (from hBCMA-A6),
the CDR1 amino acid sequence as set forth in SEQ ID NO: 13, the CDR2 amino acid sequence as set forth in SEQ ID NO: 14, and the CDR3 amino acid sequence as set forth in SEQ ID NO: 15 (from hBCMA-H4), or
the CDR1 amino acid sequence as set forth in SEQ ID NO: 31, the CDR2 amino acid sequence as set forth in SEQ ID NO: 32, and the CDR3 amino acid sequence as set forth in SEQ ID NO: 33 (from hBCMA VcMRo8 (VF7/VF8)).

42. The multivalent antibody of any one of claims 18 to 40, wherein the sdAb comprises any one of SEQ ID NOs: 40, 41, 44, 47, 50, 53 to 56, 58, 79, and 80.

43. A nucleic acid molecule encoding the multivalent antibody as defined in any one of claims 18 to 42.

44. A use of the multivalent antibody as defined in any one of claims 18 to 42 for treatment of a cancer or an autoimmune disease.

45. A method of treating a cancer or an autoimmune disease in subject comprising administering to the subject the multivalent antibody as defined in any one of claims 18 to 42.

46. A chimeric antibody receptor (CAR), which binds to human BCMA, comprising the VHH sdAb as defined in any one of claims 1 to 14.

47. The CAR of claim 46 comprising in N-terminal to C-terminal direction:
an BCMA binding domain comprising the sdAb as defined in any one of claims 1 to 14,
a polypeptide hinge,
a transmembrane domain, and
a cytoplasmic domain comprising a signaling domain, preferably wherein the cytoplasmic domain further comprises a co-stimulatory domain.

48. The CAR of claim 47, wherein the polypeptide hinge is a CD8 hinge domain, preferably comprising SEQ ID NO: 71.

49. The CAR of any claim 47 or 48, wherein the transmembrane domain is a CD28 transmembrane domain, preferably comprising SEQ ID NO: 72.

50. The CAR of any one of claims 47 to 49, wherein the signaling domain is a CD3-zeta signaling domain, preferably comprising SEQ ID NO: 74.

51. The CAR of any one of claims 47 to 50, wherein the co-stimulatory domain is a 4-1BB co-stimulatory domain, preferably comprising SEQ ID NO: 73.

52. The CAR of any of claims 47 to 51, further comprising a flexible amino acid linker between the sdAb and the polypeptide hinge, preferably comprising SEQ ID NO: 70.

53. The CAR of any one of claims 47 to 52, further comprising a signal peptide, preferably wherein the signal peptide is a signal peptide from human CD28, more preferably wherein the signal peptide from human CD28 comprises SEQ ID NO: 69.

54. The CAR of any one of claims 46 to 53, which is encoded by SEQ ID NO: 68.

55. The CAR of any one of claims 46 to 54, wherein the sdAb comprises any one of SEQ ID NOs: 40, 41, 44, 47, 50, 53 to 56, 58, 79, and 80.

56. The CAR of any one of claim 46 to 55, where in the VHH sdAb comprises:
the CDR1 amino acid sequence as set forth in SEQ ID NO: 1, the CDR2 amino acid sequence as set forth in SEQ ID NO: 2, and the CDR3 amino acid sequence as set forth in SEQ ID NO: 3 (from hBCMA-E7 or hBCMA-2C3),
the CDR1 amino acid sequence as set forth in SEQ ID NO: 4, the CDR2 amino acid sequence as set forth in SEQ ID NO: 5, and the CDR3 amino acid sequence as set forth in SEQ ID NO: 6 (from hBCMA-H2 or hBCMA-4D!),
the CDR1 amino acid sequence as set forth in SEQ ID NO: 7, the CDR2 amino acid sequence as set forth in SEQ ID NO: 8, and the CDR3 amino acid sequence as set forth in SEQ ID NO: 9 (from hBCMA-V3),
the CDR1 amino acid sequence as set forth in SEQ ID NO: 22, the CDR2 amino acid sequence as set forth in SEQ ID NO: 23, and the CDR3 amino acid sequence as set forth in SEQ ID NO: 24 (from hBCMA-A6),
the CDR1 amino acid sequence as set forth in SEQ ID NO: 13, the CDR2 amino acid sequence as set forth in SEQ ID NO: 14, and the CDR3 amino acid sequence as set forth in SEQ ID NO: 15 (from hBCMA-H4), or
the CDR1 amino acid sequence as set forth in SEQ ID NO: 31, the CDR2 amino acid sequence as set forth in SEQ ID NO: 32, and the CDR3 amino acid sequence as set forth in SEQ ID NO: 33 (hBCMA VcMRo8 (from VF7/VF8)).

57. A nucleic acid molecule encoding the CAR as defined in any one of claims 46 to 56.

58. A vector comprising the nucleic acid molecule as defined in any one of claims 15, 17, and 57.

59. The vector of claim 58, which is a viral vector.

60. The vector of claim 59, wherein the viral vector is a lentivirus vector.

61. A recombinant viral particle comprising the nucleic acid molecule as defined in any one of claims 15, 17, and 57.

62. The recombinant viral particle of claim 61, which is a recombinant lentiviral particle.

63. A cell comprising the nucleic acid molecule as defined in any one of claims 15, 17, and 57.

64. An engineered cell expressing at the cell surface membrane the CAR as defined in any one of claims 46 to 56.

65. The engineered cell of claim 64, which is an immune cell.

66. The engineered cell of claim 65, wherein the immune cell is derived from T-lymphocytes.

67. A use of the engineered cell as defined in any one of claims 64 to 66 for treatment of a cancer or an autoimmune disease.

68. The use of claim 44 or 67, wherein the cancer is a hematological malignancy, preferably wherein the hematological malignancy is characterized by aberrant or increased BCMA expression relative to healthy cells.

69. The use of claim 68, wherein the hematological malignancy is multiple myeloma (MM), lymphoma, chronic lymphocytic leukemia (CLL), B-cell acute lymphoblastic leukemia (B-ALL), or acute myelogenous leukemia (AML).

70. A method of treating a cancer in a subject, comprising administering to the subject the engineered cell as defined in any one of claims 64 to 66.

71. The method of claim 45 or 70, wherein the cancer is a hematological malignancy, preferably wherein the hematological malignancy is characterized by aberrant or increased BCMA expression relative to healthy cells.

72. The method of claim 71, wherein the hematological malignancy is multiple myeloma (MM), lymphoma, chronic lymphocytic leukemia (CLL), B-cell acute lymphoblastic leukemia (B-ALL), or acute myelogenous leukemia (AML).


SEQUENCE LISTING (82 sequences)

SEQ ID NO: 1 - protein, 8 aa, synthetic construct
  REGION 1..8: note = hBCMA-E7 CDR1
GRATDHFV

SEQ ID NO: 2 - protein, 8 aa, synthetic construct
  REGION 1..8: note = hBCMA-E7 CDR2
RVWSGGSP

SEQ ID NO: 3 - protein, 13 aa, synthetic construct
  REGION 1..13: note = hBCMA-E7 CDR3
AATKDIMSRSYDY

SEQ ID NO: 4 - protein, 8 aa, synthetic construct
  REGION 1..8: note = hBCMA-H2 CDR1
GRTSNNFV

SEQ ID NO: 5 - protein, 8 aa, synthetic construct
  REGION 1..8: note = hBCMA-H2 CDR2
RVWSGSTP

SEQ ID NO: 6 - protein, 13 aa, synthetic construct
  REGION 1..13: note = hBCMA-H2 CDR3
AATKDIMSRSYDY

SEQ ID NO: 7 - protein, 8 aa, synthetic construct
  REGION 1..8: note = hBCMA-V3 CDR1
GRASNHFV

SEQ ID NO: 8 - protein, 8 aa, synthetic construct
  REGION 1..8: note = hBCMA-V3 CDR2
RVWSGGSP

SEQ ID NO: 9 - protein, 13 aa, synthetic construct
  REGION 1..13: note = hBCMA-V3 CDR3
AATKDIMSRSYDY

SEQ ID NO: 10 - protein, 8 aa, synthetic construct
  REGION 1..8: note = hBCMA-B5 CDR1
GSIFGTYN

SEQ ID NO: 11 - protein, 7 aa, synthetic construct
  REGION 1..7: note = hBCMA-B5 CDR2
ISSAGNT

SEQ ID NO: 12 - protein, 14 aa, synthetic construct
  REGION 1..14: note = hBCMA-B5 CDR3
NGAPWADAEVKVYN

SEQ ID NO: 13 - protein, 8 aa, synthetic construct
  REGION 1..8: note = hBCMA-H4 CDR1
GDSFGAYA

SEQ ID NO: 14 - protein, 7 aa, synthetic construct
  REGION 1..7: note = hBCMA-H4 CDR2
ISSAGNT

SEQ ID NO: 15 - protein, 14 aa, synthetic construct
  REGION 1..14: note = hBCMA-H4 CDR3
NGAPWADEPVKVWN

SEQ ID NO: 16 - protein, 8 aa, synthetic construct
  REGION 1..8: note = hBCMA-H1 CDR1
GSGFGTHV

SEQ ID NO: 17 - protein, 7 aa, synthetic construct
  REGION 1..7: note = hBCMA-H1 CDR2
ISSAGST

SEQ ID NO: 18 - protein, 14 aa, synthetic construct
  REGION 1..14: note = hBCMA-H1 CDR3
NGAPWADEPVKVWN

SEQ ID NO: 19 - protein, 8 aa, synthetic construct
  REGION 1..8: note = hBCMA-F2 CDR1
GNSFGAYN

SEQ ID NO: 20 - protein, 7 aa, synthetic construct
  REGION 1..7: note = hBCMA-F2 CDR2
ISSTGNT

SEQ ID NO: 21 - protein, 14 aa, synthetic construct
  REGION 1..14: note = hBCMA-F2 CDR3
NGAPWGDDPVKVWS

SEQ ID NO: 22 - protein, 8 aa, synthetic construct
  REGION 1..8: note = hBCMA-A6 CDR1
GSIFDAYN

SEQ ID NO: 23 - protein, 7 aa, synthetic construct
  REGION 1..7: note = hBCMA-A6 CDR2
ISSAGTT

SEQ ID NO: 24 - protein, 14 aa, synthetic construct
  REGION 1..14: note = hBCMA-A6 CDR3
NGAPWGDAPVKVED

SEQ ID NO: 25 - protein, 8 aa, synthetic construct
  REGION 1..8: note = hBCMA VcMRo1(V1/V6) CDR1
GDPITVAT

SEQ ID NO: 26 - protein, 7 aa, synthetic construct
  REGION 1..7: note = hBCMA VcMRo1(V1/V6) CDR2
ISSAGST

SEQ ID NO: 27 - protein, 14 aa, synthetic construct
  REGION 1..14: note = hBCMA VcMRo1(V1/V6) CDR3
NGAPWGDAPVKVWT

SEQ ID NO: 28 - protein, 8 aa, synthetic construct
  REGION 1..8: note = hBCMA-D2 CDR1
GSIFGTYN

SEQ ID NO: 29 - protein, 7 aa, synthetic construct
  REGION 1..7: note = hBCMA-D2 CDR2
ISSAGNT

SEQ ID NO: 30 - protein, 14 aa, synthetic construct
  REGION 1..14: note = hBCMA-D2 CDR3
NGAPWGDALVKVWN

SEQ ID NO: 31 - protein, 8 aa, synthetic construct
  REGION 1..8: note = hBCMA-V8 CDR1
GTIFSRNA

SEQ ID NO: 32 - protein, 7 aa, synthetic construct
  REGION 1..7: note = hBCMA-V8 CDR2
ITIGGTT

SEQ ID NO: 33 - protein, 16 aa, synthetic construct
  REGION 1..16: note = hBCMA-V8 CDR3
NADPEGSWNWVRRGDY

SEQ ID NO: 34 - protein, 8 aa, synthetic construct
  REGION 1..8: note = hBCMA-2F10 CDR1
GHTTKNFV

SEQ ID NO: 35 - protein, 8 aa, synthetic construct
  REGION 1..8: note = hBCMA-2F10 CDR2
RVWSGGSP

SEQ ID NO: 36 - protein, 13 aa, synthetic construct
  REGION 1..13: note = hBCMA-2F10 CDR3
AATKDIMSRGYVY

SEQ ID NO: 37 - protein, 8 aa, synthetic construct
  REGION 1..8: note = hBCMA-3F2 CDR1
GHTSNQFV

SEQ ID NO: 38 - protein, 8 aa, synthetic construct
  REGION 1..8: note = hBCMA-3F2 CDR2
RFWGGGSP

SEQ ID NO: 39 - protein, 13 aa, synthetic construct
  REGION 1..13: note = hBCMA-3F2 CDR3
AATKDILSRGYDY

SEQ ID NO: 40 - protein, 120 aa, synthetic construct
  REGION 1..120: note = hBCMA-E7
QVQLVESGGGLVQTGDSLRLACTISGRATDHFVMAWFRRAPGKEREYVATRVWSGGSPYYLDSVKGRFAIAIDNAKNTAYLQMNNLKPEDTAVYYCAATKDIMSRSYDYWGLGTQVTVSS

SEQ ID NO: 41 - protein, 120 aa, synthetic construct
  REGION 1..120: note = hBCMA-H2
QVKAEESGGGLVRPGDSLRLTCTISGRTSNNFVMAWFRRTPGKEREYVATRVWSGSTPYYHDSVKGRFTISIDDDKNTAYLQMNSLKPEDTAVYYCAATKDIMSRSYDYWGLGTQVTVSS

SEQ ID NO: 42 - protein, 120 aa, synthetic construct
  REGION 1..120: note = hBCMA-V3 (hBCMA-VcMRo3)
EVQLEQSGGGLVQTGDSLRLTCTIPGRASNHFVMAWFRRAPGKEREFVATRVWSGGSPYYSDSVKGRFAIAIDNAKNTAYLQMNSLKPEDTAVYYCAATKDIMSRSYDYWGLGTQVTVSS

SEQ ID NO: 43 - protein, 120 aa, synthetic construct
  REGION 1..120: note = hBCMA-B5
QVKLEESGGGLVQPGGSLRLSCAASGSIFGTYNMGWYRQAPGKQRELVAAISSAGNTFYRDSVKGRFTVSRDNAKNTVYLQMDRLKYEDTAVYNCNGAPWADAEVKVYNWGQGTQVTVSS

SEQ ID NO: 44 - protein, 120 aa, synthetic construct
  REGION 1..120: note = hBCMA-H4
QVQLVESGGGLVQPGGSLRLSCAASGDSFGAYAMGWYRQAPGKQRELVAAISSAGNTFYRDSVKGRFTVSRNNAKNAMYLQMDRLKPEDTAVYQCNGAPWADEPVKVWNWGLGTQVTVSS

SEQ ID NO: 45 - protein, 120 aa, synthetic construct
  REGION 1..120: note = hBCMA-H1
QVQLVESGGGLVQPGGSLRLSCAASGSGFGTHVMGWYRQAPGKPRELVAAISSAGSTFYRDSVKGRFTVSRDNAKNTMYLQMDRLKPEDTAVYYCNGAPWADEPVKVWNWGQGTQVTVSS

SEQ ID NO: 46 - protein, 120 aa, synthetic construct
  REGION 1..120: note = hBCMA-F2
QVKLEESGGGLVKPGGSLRLSCGASGNSFGAYNMGWYRQAPGKQRELVAAISSTGNTFYRDSVRGRFTVSRDNAKSTMSLQMERLKPEDTAVYLCNGAPWGDDPVKVWSWGQGTQVTVSS

SEQ ID NO: 47 - protein, 120 aa, synthetic construct
  REGION 1..120: note = hBCMA-A6
QVQLVESGGGLVQPGGSLRLSCVASGSIFDAYNMGWYRQAPGKQRELVAAISSAGTTFYRDSVKGRFTVSRNNAKNTMYLQMDRLRPEDTAVYDCNGAPWGDAPVKVEDWGQGTQVTVSS

SEQ ID NO: 48 - protein, 120 aa, synthetic construct
  REGION 1..120: note = hBCMA VcMRo1(V1/V6)
EVQLQQSGGGLVQAGESLTISCAVFGDPITVATMGWYRQAPGKLRELVAAISSAGSTFYRDSVRGRFTVSRDNAKSTMYLQMDRLKVEDTAVYSCNGAPWGDAPVKVWTWGEGTQVTVSS

SEQ ID NO: 49 - protein, 122 aa, synthetic construct
  REGION 1..122: note = hBCMA-D2
QAQVQLVESGGGLVQPGGSLRLSCAASGSIFGTYNMGWYRQAPGKQRELVAAISSAGNTFYRDSVKGRFTVSRDNAKNTMYLQMDRLKYEDTAVYNCNGAPWGDALVKVWNWGQGTQVTVSS

SEQ ID NO: 50 - protein, 121 aa, synthetic construct
  REGION 1..121: note = hBCMA-V8 (VcMRo8 (VF7/VF8))
QVQLVESGGGLVQPGGSLRLSCAASGTIFSRNALAWFRQAPGKQRELVAHITIGGTTVYKDSVKGRFTISRDNAKNTVYLQMDALKPEDTAVYYCNADPEGSWNWVRRGDYWGQGTQVTVS

SEQ ID NO: 51 - protein, 120 aa, synthetic construct
  REGION 1..120: note = hBCMA-2F10
QVQLVESGGGLVQSGNSLRLSCSISGHTTKNFVMAWFRRAPGKERAIVATRVWSGGSPWYSESAKGRFTISIDDARNTAYLQMNNLKPEDTAVYYCAATKDIMSRGYVYWGLGTQVTVSS

SEQ ID NO: 52 - protein, 120 aa, synthetic construct
  REGION 1..120: note = hBCMA-3F2
QVQLVESGGRLVQTGDSLRLTCEISGHTSNQFVLAWFRRPPGKEREVVATRFWGGGSPYYSDSVRGRFAIAIDDAKNTAYLQMSSLKPEDTAVYYCAATKDILSRGYDYWGQGTQVTVSS

SEQ ID NO: 53 - protein, 120 aa, synthetic construct; identical to SEQ ID NO: 47
  REGION 1..120: note = hBCMA-A6 used in CAR

SEQ ID NO: 54 - protein, 120 aa, synthetic construct
  REGION 1..120: note = hBCMA-E7 used in CAR
QVQLVESGGGLVRPGDSLRLTCTISGRTSNNFVMAWFRRTPGKEREYVATRVWSGSTPYYHDSVKGRFTISIDDDKNTAYLQMNSLKPEDTAVYYCAATKDIMSRSYDYWGQGTQVTVSS

SEQ ID NO: 55 - protein, 120 aa, synthetic construct; identical to SEQ ID NO: 54
  REGION 1..120: note = BCMA-H2 used in CAR

SEQ ID NO: 56 - protein, 120 aa, synthetic construct; identical to SEQ ID NO: 44
  REGION 1..120: note = BCMA-H4 used in CAR

SEQ ID NO: 57 - protein, 120 aa, synthetic construct
  REGION 1..120: note = BCMA-V3 used in CAR
QVQLVESGGGLVQTGDSLRLTCTIPGRASNHFVMAWFRRAPGKEREFVATRVWSGGSPYYSDSVKGRFAIAIDNAKNTAYLQMNSLKPEDTAVYYCAATKDIMSRSYDYWGLGTQVTVSS

SEQ ID NO: 58 - protein, 122 aa, synthetic construct
  REGION 1..122: note = BCMA-V8 used in CAR
QVQLVESGGGLVQPGGSLRLSCAASGTIFSRNALAWFRQAPGKQRELVAHITIGGTTVYKDSVKGRFTISRDNAKNTVYLQMDALKPEDTAVYYCNADPEGSWNWVRRGDYWGQGTQVTVSS

SEQ ID NO: 59-60 - skipped (2 entries)

SEQ ID NO: 61 - protein, 13 aa, synthetic construct
  REGION 1..13: note = Group 1 CDR3 Consensus
  SITE 7: note = MISC_FEATURE - M or L
  SITE 10: note = MISC_FEATURE - S or G
  SITE 12: note = MISC_FEATURE - D or V
AATKDIXSRXYXY

SEQ ID NO: 62 - skipped

SEQ ID NO: 63 - protein, 7 aa, synthetic construct
  REGION 1..7: note = Group 2 CDR2 Consensus
  SITE 6: note = MISC_FEATURE - N or S
ISSAGXT

SEQ ID NO: 64 - protein, 14 aa, synthetic construct
  REGION 1..14: note = Group 2 CDR3 Consensus
  SITE 8: note = MISC_FEATURE - A or E
  SITE 9: note = MISC_FEATURE - E or P
  SITE 13: note = MISC_FEATURE - Y or W
NGAPWADXXVKVXN

SEQ ID NO: 65 - skipped

SEQ ID NO: 66 - protein, 7 aa, synthetic construct
  REGION 1..7: note = Group 3 CDR2 Consensus
  SITE 4: note = MISC_FEATURE - T or A
  SITE 6: note = MISC_FEATURE - N, T, or S
ISSXGXT

SEQ ID NO: 67 - protein, 14 aa, synthetic construct
  REGION 1..14: note = Group 3 CDR3 Consensus
  SITE 8: note = MISC_FEATURE - D or A
  SITE 9: note = MISC_FEATURE - P or L
  SITE 13: note = MISC_FEATURE - W or E
  SITE 14: note = MISC_FEATURE - S, D, T, or N
NGAPWGDXXVKVXX

SEQ ID NO: 68 - DNA, 1675 bp, synthetic construct
  source 1..1675: mol_type = other DNA
  misc_feature 1..1675: note = Example CAR Modular Construct
  misc_feature 62..63: note = sdAb sequence inserted between these sites
atgctcaggctgctcttggctctcaacttattcccttcaattcaagtaacaggagggtcttcgaagacttcctttgcgagacgacggtggcgggggatcaggtggtggaggtagcgggggagggggctcaggcggtacaactacgcctgcacctcgcccaccgaccccagcaccaaccatcgcttcacagcctttgagcctgcgaccagaggcatgtcgccctgctgcgggcggtgccgttcatactcgcggacttgattttgcgtgtgacgtcgtctcgccttctaagcccttttgggtgctggtggtggttggtggagtcctggcttgctatagcttgctagtaacagtggcctttattattttctgggtgaggaaacggggcagaaagaaactcctgtatatattcaaacaaccatttatgcgaccagtacaaactactcaagaggaagatggctgtagctgccgatttccagaagaagaagaaggaggatgtgaactgctgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgcgctagcgccacgaacttctctctgttaaagcaagcaggcgacgtggaagaaaaccccggtcccatggtgagcaagggcgaggaggacaacatggccagcctgcccgccacccacgagctgcacatcttcggcagcatcaacggcgtggacttcgacatggtgggccagggcaccggcaaccccaacgacggctacgaggagctgaacctgaagagcaccaagggcgacctgcagttcagcccctggattctggtgccccacatcggctacggcttccaccagtacctgccctaccccgacggcatgagccccttccaggccgccatggtggacggcagcggctaccaggtgcacaggaccatgcagttcgaggacggcgccagcctgaccgtgaactacaggtacacctacgagggcagccacatcaagggcgaggcccaggtgaagggcaccggcttccccgccgacggccccgtgatgaccaacagcctgaccgccgccgactggtgcaggagcaaaaagacctaccccaacgacaagaccatcatcagcaccttcaagtggagctacaccaccggcaacggcaagaggtacaggagcaccgccaggaccacctacaccttcgccaagcccatggccgccaactacctgaagaaccagcccatgtacgtgttcagaaagaccgagctgaagcacagcaagaccgagctgaacttcaaggagtggcagaaggccttcaccgacgtgatgggcatggacgagctgtacaagcccaagaagaagaggaaggtggaggacccccccgccgccaagagggtgaagctggactaa

SEQ ID NO: 69 - protein, 18 aa, synthetic construct
  REGION 1..18: note = Human CD28 Signal Peptide
MLRLLLALNLFPSIQVTG

SEQ ID NO: 70 - protein, 17 aa, synthetic construct
  REGION 1..17: note = Synthetic Flexible Linker Domain
GGGGSGGGGSGGGGSGG

SEQ ID NO: 71 - protein, 45 aa, synthetic construct
  REGION 1..45: note = Human CD8 Hinge Domain
TTTPAPRPPTPAPTIASQPLSLRPEACRPAAGGAVHTRGLDFACD

SEQ ID NO: 72 - protein, 32 aa, synthetic construct
  REGION 1..32: note = Human CD28 Transmembrane Domain
PSKPFWVLVVVGGVLACYSLLVTVAFIIFWVR

SEQ ID NO: 73 - protein, 42 aa, synthetic construct
  REGION 1..42: note = Human 4-1BB Costimulatory Domain
KRGRKKLLYIFKQPFMRPVQTTQEEDGCSCRFPEEEEGGCEL

SEQ ID NO: 74 - protein, 114 aa, synthetic construct
  REGION 1..114: note = Human CD3zeta Signaling Domain
LRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 75 - protein, 5 aa, synthetic construct
  REGION 1..5: note = Short Flexible Linker
GGGGS

SEQ ID NO: 76 - DNA, 858 bp, synthetic construct
  source 1..858: mol_type = other DNA
  misc_feature 1..858: note = Example Bispecific T cell Engager Modular Construct DNA Sequence
  misc_feature 72..73: note = sdAb coding sequence inserted between these sites
atggagtttgggctgagctgggttttcctcgttgctctttttagaggtgtccagtgtacaggagggtcttcggaagacttccttggaggaggcggaagtcaagtgcagttgcaacaaccaggtgccgagcttgggaaaccgggtaccagcgttaaactctcttgcaaagcatccggttatacctttacttcttactggatccattgggtcaaacaacgcccaggccagggtctcgaatgggtgggcaacatcaacccaaacagcggctctatcaattataacgaaaagtttaagaataaagcaactctcacggtggataagagcagctcaaccgcttatatgcaattgagtactttgaccagtgaagatttcgctgtgtattattgcacgcgcgatacatccggacagtattatttcgattattggggccaggggacaaccctgacagtgtctagcggcggtggtgggtcaggcggcggtgggagcggaggaggtggaagcgatatcgtaatgagtcaatcccctagcagcttggcggtaagtgctggggaacgcgttacaatgtcctgtaagagctctcaatctttgctgaattccaggactaggaagaattatctggcctggtatcaacaaaagcccggacaaagtccaaagctcctgatttattgggctagcaccagagagtcaggagtaccagaccggtttactggaagcgggtcaggaaccgactttaccctgacaatatccagcgtccaagcagaagatcttgccgtgtactattgtattcagtcctaccacttgcgcacttttggggggggaacaaaactcgagataaagcaccaccaccaccaccactag

SEQ ID NO: 77 - protein, 499 aa, synthetic construct
  REGION 1..499: note = BCMA-A6-H4-BBz CAR (Example Multi-binder CAR)
  REGION 19..148: note = A6 sdAb
  REGION 155..264: note = H4 sdAb
MLRLLLALNLFPSIQVTGQVQLVESGGGLVRPGDSLRLTCTISGRTSNNFVMAWFRRTPGKEREYVATRVWSGSTPYYHDSVKGRFTISIDDDKNTAYLQMNSLKPEDTAVYYCAATKDIMSRSYDYWGQGTQVTVSSPSGGGGQVQLVESGGGLVQPGGSLRLSCAASGDSFGAYAMGWYRQAPGKQRELVAAISSAGNTFYRDSVKGRFTVSRNNAKNAMYLQMDRLKPEDTAVYQCNGAPWADEPVKVWNWGLGTQVTVSSPSTTTPAPRPPTPAPTIASQPLSLRPEACRPAAGGAVHTRGLDFACDPSKPFWVLVVVGGVLACYSLLVTVAFIIFWVRKRGRKKLLYIFKQPFMRPVQTTQEEDGCSCRFPEEEEGGCELLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 78 - protein, 209 aa, synthetic construct
  REGION 203..204: note = positions separated by a CD3-specific scFv
  REGION 22..141: note = BCMA H4 sdAb
MEFGLSWVFLVALFRGVQCTGQVQLVESGGGLVQPGGSLRLSCAASGDSFGAYAMGWYRQAPGKQRELVAAISSAGNTFYRDSVKGRFTVSRNNAKNAMYLQMDRLKPEDTAVYQCNGAPWADEPVKVWNWGLGTQVTVSSGGGGSGGGGSGGGGSGGTTTPAPRPPTPAPTIASQPLSLRPEACRPAAGGAVHTRGLDFACDHHHHHH

SEQ ID NO: 79 - protein, 120 aa, synthetic construct
QVKLEESGGGLVQTGDSLRLACTISGRATDHFVMAWFRRAPGKEREYVATRVWSGGSPYYLDSAKGRFAIAIDNAKNTAYLQMNNLKPEDTAVYYCAATKDIMSRSYDYWGLGTQVTVSS

SEQ ID NO: 80 - protein, 120 aa, synthetic construct
QVQLVESGGGLVRPGDSLRLTCTISGRTSNNFVMAWFRRTPGKEREYVATRVWSGSTPYYHDSVKGRFTISIDDDKNTAYLQMNSLKPEDTAVYYCAATKDIMSRSYDYWGLGTQVTVSS

SEQ ID NO: 81 - DNA, 34 bp, synthetic construct
  source 1..34: mol_type = other DNA
catgtgtagactcgcggcccagccggccatggcc

SEQ ID NO: 82 - DNA, 47 bp, synthetic construct
  source 1..47: mol_type = other DNA
catgtgtagattcctggccggcctggcctgaggagacggtgacctgg